(19) United States
(12) Patent Application Publication
(10) Pub. No.: US 2018/0016596 A1
(43) Pub. Date: Jan. 18, 2018

(54) NUCLEIC ACID CONSTRUCTS, PLANTS COMPRISING SAME AND USES THEREOF IN ENHANCING PLANT PEST RESISTANCE AND ALTERING PLANT MONOTERPENE PROFILE

(71) Applicant: Futuragene Israel Ltd., Rehovot (IL)

(72) Inventors: Dror AVISAR, Kochav Yair (IL); Miron ABRAMSON, Karmei Yosef (IL); Tany SINAI, Neve Yarak (IL); Roee SHAVIT, Rehovot (IL)

(73) Assignee: Futragene Israel Ltd., Rehovot (IL)

(21) Appl. No.: 15/540,061

(22) PCT Filed: Dec. 29, 2015
(86) PCT No.: PCT/IL2015/051266

§ 371 Date: Jun. 27, 2017

Related U.S. Application Data
(60) Provisional application No. 62/097,186, filed on Dec. 29, 2014.

(51) Int. Cl. C12N 15/82; A01N 43/90; C12N 9/16; C12N 9/04

(52) U.S. Cl. CPC C12N 15/8286; C12Y 101/01183; C12N 15/8257; C12N 15/8247; C12Y 301/07011; C12N 9/0006; A01N 43/90; C12N 9/16

(57) ABSTRACT

Nucleic acid constructs encoding Geraniol Synthase (GS), Geraniol Reductase (GR), Geraniol Dehydrogenase (GD), and/or Citral Reductase (CR) and plants comprising same are provided.

### FIELD AND BACKGROUND OF THE INVENTION

The present invention, in some embodiments thereof, relates to nucleic acid constructs, plants comprising same and uses thereof in enhancing plant pest resistance and altering plant monoterpene profile.

Eucalyptus species are commercially important woody plants used for industrial purposes such as essential oil production, wood pulp, wood pellets, charcoal and biomass fuel. Pest infestations, such as Gall wasp Leptocybe invasa, the red gum lerp psyllid, Glycaspis brimblecombei and Thaumastocoris peregrinus, have been identified and pose a threat to natural populations as well as cultivated eucalyptus such as in Australia, Africa, South and North America, China, India and the Mediterranean. Symptoms of Glycaspis brimblecombei infestation, for example, include leaf loss and drying of leaf shoots while severe infestation can cause complete defoliation and death of trees. Efforts to control pest infections of eucalyptus have included attempts to isolate naturally resistant plants and natural predators; however these efforts have met with limited or no success.

Monoterpenes are plant volatile compounds which are the main constituents of essential oils of plants. Many monoterpenes contribute to the aromatic profile of plants and some are used as natural sources of aromas to add flavor and fragrance to foods and cosmetics. Monoterpenes also play major ecological and physiological roles in flower pollination and in responses to biotic or abiotic stress [Gutenshon et al. The Plant Journal (2013) 75: 351-363; Yang et al. Metabolic Engineering (2011) 13: 414-425]. Monoterpenes are known to act as natural pesticides in plants and have been used effectively to control pre-harvest and postharvest phytophagous pests and as pest repellents for biting flies and for home and garden pests [Regnault-Roger et al., Annu Rev Entomol. (2012) 57: 405-24].

While the backbones of the biosynthetic pathways leading to production of monoterpenes are ubiquitous to all plant species, the composition of terpenes often differs dramatically between species or even varieties leading to the diversity of volatile compounds in general as well as aroma and flavors among herbs and fruits.

This diversity seems to stem mainly from the specific composition and expression of the key-enzymes in the biosynthetic pathway, the terpene synthases, and additional downstream modification enzymes. All terpenoids are derived from the universal five-carbon building blocks isopentenyl pyrophosphate (IPP) and its isomer dimethylallyl pyrophosphate (or dimethylallyl diphosphate, DMAPP). Geranyl pyrophosphate (also known as geranyl diphosphate, GPP), the precursor of most monoterpenes, is synthesized in plastids from DMAPP and IPP by GPP synthases (GPPSs) [Ohara et al. Plant Biotechnology Journal (2010) 8: 28-37].

In the past years a large variety of monoterpene synthase genes from different species have been characterized and many of them were used for metabolic engineering of plants. Production and increased emissions of target monoterpenes through heterologous expression of the corresponding terpenoid synthase genes, has been achieved in various plants; for example geraniol in tomato fruits (Davidovich-Rikanati et al., 2007); linalool in Arabidopsis [Aharoni et al., Plant Cell (2003) 15: 2866-2884]; b-pinene and g-terpinene in tobacco [Lucker et al., Plant Physiology (2004) 134: 510-519]; and limonene in mint [Diemer et al., Plant Physiology and Biochemistry (2001) 39: 603-6141.].

Geraniol synthase (GS), Geraniol reductase (GR) and Geraniol dehydrogenase (GD) lead to the production of monoterpenes that are found in minimal or undetectable quantities in most eucalyptus species. GS converts GPP to geraniol, GR converts geraniol to citronellol and GD converts geraniol to geranial, and citronellol to citronellal (FIG. 1).

Geraniol, geranial, neral, citronellol and citronellal have been found to confer pest resistance in different crops [see e.g. Chen and Viljoen, South African Journal of Botany (2010) 76: 643-651]. However, increased content of geraniol and geranial by heterologous expression of GS in maize had no effect on fungal resistance [Yang et al. Metabolic Engineering (2011) 13: 414-425]. While absent in most Eucalyptus species, citronellal is the major essential oil (approximately 70%) in Corymbia citriodora, having a monoterpene profile significantly different than the genus Eucalyptus. Corymbia citriodora has been shown to be resistant to certain pests, such as gall wasp Leptocybe invasa, the lerp psyllid Glycaspis brimblecombei and the Snout weevil beetle Gonipterus scutellatus, [see e.g. Batish et al., Z Naturforsch C. (2006) 61(7-8): 465-71 and Thu et al. ScienceAsia 35 (2009): 113-117].

### ADDITIONAL RELATED ART

U.S. Pat. No. 8,124,375;

U.S. Pat. No. 6,515,202;

U.S. Pat. No. 7,985,567;

U.S. Pat. No. 8,329,438;

U.S. Pat. No. 6,291,745;

International Patent Application Publication Number WO2000036081A2;

Iijima et al. [Archives of Biochemistry and Biophysics (2006) 448: 141-149];

Stott et al. [The Journal of Biological Chemistry (1993) 268(9): 6097-6106];

Yuan et al. [Nat. Prod. Bioprospect. (2011) 1: 108-111]; and

Lange and Ahkami [Plant Biotechnology Journal (2013) 11: 169-196].

### SUMMARY OF THE INVENTION

According to an aspect of some embodiments of the present invention there is provided a genetically modified woody plant comprising a heterologous nucleic acid sequence encoding at least one polypeptide selected from the group consisting of Geraniol Synthase (GS), Geraniol Reductase (GR), Geraniol Dehydrogenase (GD) and Citral Reductase (CR).

According to an aspect of some embodiments of the present invention there is provided a nucleic acid construct comprising a nucleic acid sequence encoding at least two polypeptides selected from the group consisting of Geraniol Synthase (GS), Geraniol Reductase (GR), Geraniol Dehydrogenase (GD) and Citral Reductase (CR) and at least one cis-acting regulatory element for directing expression of the nucleic acid sequence.

According to an aspect of some embodiments of the present invention there is provided a nucleic acid construct system comprising at least two nucleic acid constructs expressing at least two polypeptides selected from the group consisting of Geraniol Synthase (GS), Geraniol Reductase (GR), Geraniol Dehydrogenase (GD) and Citral Reductase (CR).

According to an aspect of some embodiments of the present invention there is provided a nucleic acid construct comprising a nucleic acid sequence encoding Geraniol Dehydrogenase (GD) and a cis-acting regulatory element for directing expression of the nucleic acid sequence in a plant cell.

According to an aspect of some embodiments of the present invention there is provided a nucleic acid construct comprising a nucleic acid sequence encoding Citral Reductase (CR) and a cis-acting regulatory element for directing expression of the nucleic acid sequence in a plant cell, wherein the nucleic acid sequence further comprises a chloroplast leader peptide.

According to an aspect of some embodiments of the present invention there is provided a nucleic acid construct comprising a nucleic acid sequence encoding Geraniol Reductase (GR) and a cis-acting regulatory element for directing expression of the nucleic acid sequence in a plant cell, wherein the nucleic acid sequence is devoid of a peroxisome C-terminus tri-amino acid signal (SRL) and comprises a chloroplast leader peptide.

According to an aspect of some embodiments of the present invention there is provided an isolated cell comprising the nucleic acid construct of some embodiments of the invention.

According to an aspect of some embodiments of the present invention there is provided an isolated plant cell comprising the nucleic acid construct of some embodiments of the invention.

According to an aspect of some embodiments of the present invention there is provided a genetically modified woody plant comprising the nucleic acid construct of some embodiments of the invention.

According to an aspect of some embodiments of the present invention there is provided a pesticidal composition, comprising as an active ingredient the nucleic acid construct or construct system of some embodiments of the invention and an agriculturally acceptable carrier or diluent.

According to an aspect of some embodiments of the present invention there is provided a method of enhancing resistance of a woody plant to pest infection, the method comprising expressing in the woody plant at least one recombinant polypeptide selected from the group consisting of Geraniol Synthase (GS), Geraniol Reductase (GR), Geraniol Dehydrogenase (GD) and Citral Reductase (CR), thereby enhancing the resistance of the woody plant to pest infection.

According to an aspect of some embodiments of the present invention there is provided a method of enhancing at least one of geraniol, geranial, neral, citronellol, citronellal and citral oil content of a woody plant, the method comprising expressing in the woody plant at least one recombinant polypeptide selected from the group consisting of Geraniol Synthase (GS), Geraniol Reductase (GR), Geraniol Dehydrogenase (GD) and Citral Reductase (CR), thereby enhancing at least one of geraniol, geranial, neral, citronellol, citronellal and citral oil content of the woody plant.

According to an aspect of some embodiments of the present invention there is provided a method of producing oil, the method comprising providing the genetically modified woody plant of some embodiments of the invention and extracting the oil from the woody plant, thereby producing oil.

According to some embodiments of the invention, the method further comprises purifying a monoterpene fraction from the oil following the extracting.

According to an aspect of some embodiments of the present invention there is provided an oil produced according to the method of some embodiments of the invention.

According to an aspect of some embodiments of the present invention there is provided eucalyptus oil having an increased content of at least one monoterpene selected from the group consisting of geraniol, geranial, neral, citronellol, citronellal and citral; as compared to a eucalyptus oil of a non-genetically modified eucalyptus.

According to some embodiments of the invention, the eucalyptus oil has a reduced content of at least one monoterpene not selected from the group consisting of geraniol, geranial, neral, citronellol, citronellal and citral; as compared to a eucalyptus oil of a non-genetically modified eucalyptus.

According to an aspect of some embodiments of the present invention there is provided a method of producing at least one monoterpene selected form the group consisting of geraniol, geranial, neral, citronellol, citronellal and citral, the method comprising providing the genetically modified woody plant of some embodiments of the invention, and extracting the monoterpene from the woody plant, thereby producing at least one monoterpene selected form the group consisting of geraniol, geranial, neral, citronellol, citronellal and citral.

According to some embodiments of the invention, the cis-acting regulatory element comprises a promoter sequence.

According to some embodiments of the invention, the promoter sequence is a constitutive promoter.

According to some embodiments of the invention, the constitutive promoter is selected from the group consisting of Cauliflower mosaic virus (CaMV) 35S promoter, Figwort mosaic virus subgenomic transcript (sgFiMV) promoter and Strawberry vein banding virus (SVBV) promoter.

According to some embodiments of the invention, the genetically modified woody plant of some embodiments of the invention is resistant to pest infection.

According to some embodiments of the invention, the monoterpene fraction comprises at least one of geraniol, geranial, neral, citronellol, citronellal and citral.

According to some embodiments of the invention, the at least one polypeptide comprises GS.

According to some embodiments of the invention, the at least one polypeptide comprises at least two polypeptides.

According to some embodiments of the invention, the at least two polypeptides comprise GS and GR.

According to some embodiments of the invention, the at least two polypeptides comprise GS and CR.

According to some embodiments of the invention, the at least two polypeptides comprise GS and GD.

According to some embodiments of the invention, the at least two polypeptides comprise GS, GR and GD.

According to some embodiments of the invention, the at least two polypeptides comprise GS, GD and CR.

According to some embodiments of the invention, the at least two polypeptides comprise GS, GR, GD and CR.

According to some embodiments of the invention, the polypeptide further comprises a chloroplast leader peptide.

According to some embodiments of the invention, the plant is a woody plant.

According to some embodiments of the invention, the woody plant is Eucalyptus or poplar.

According to some embodiments of the invention, the pest is selected from the group consisting of Glycaspis brimblecombei, Thaumastocoris peregrinus, Leptocybe invasa and Ophelimus maskelli.

According to some embodiments of the invention, the GS nucleic acid sequence comprises SEQ ID NOs: 36-46.

According to some embodiments of the invention, the GR nucleic acid sequence comprises SEQ ID NOs: 47-57, 82, 84, 86, 88 or 90.

According to some embodiments of the invention, the GD nucleic acid sequence comprises SEQ ID NOs: 58-63.

According to some embodiments of the invention, the CR nucleic acid sequence comprises SEQ ID NO: 80.

According to some embodiments of the invention, the GS amino acid sequence comprises SEQ ID NOs: 1-12.

According to some embodiments of the invention, the GR amino acid sequence comprises SEQ ID NOs: 13-29, 81, 83, 85, 87 or 89.

According to some embodiments of the invention, the GD amino acid sequence comprises SEQ ID NOs: 30-35.

According to some embodiments of the invention, the CR amino acid sequence comprises SEQ ID NO: 79.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a scheme of a monoterpene modification pathway, according to some embodiments of the invention, illustrating the compounds and the respective enzymes (marked in grey).

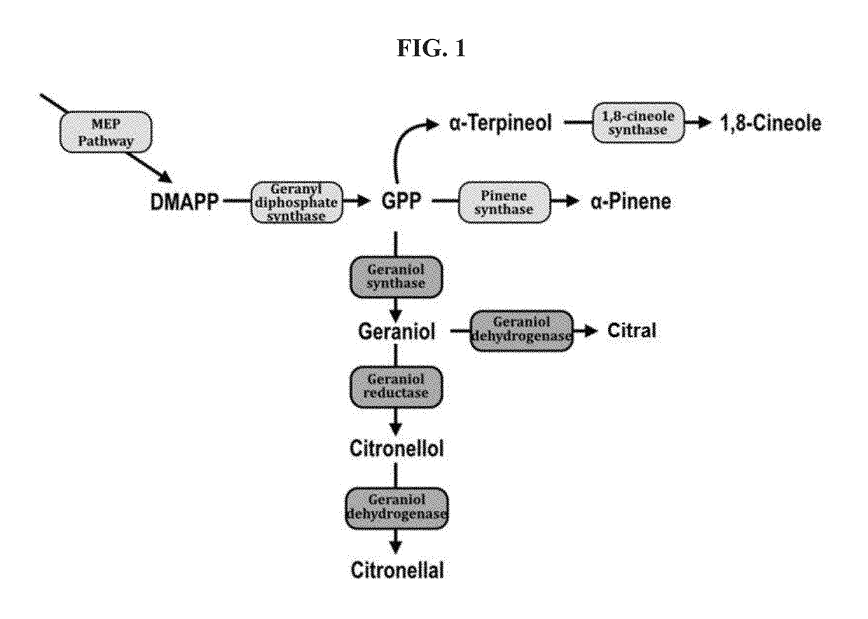

FIG. 2 is a picture demonstrating the free choice experiment for testing the effect of geraniol and citronellal on resistance of Eucalyptus camaldulensis to Ophelimus maskelli infection. Each cage contained four Eucalyptus camaldulensis saplings and each sapling had 10 disks attached to its branches with one of the treatments: geraniol, citronellol, eucalyptol control or mineral oil control. In the next step, Ophelimus maskelli wasps were released in the middle the cage.

FIG. 3 is a bar graph showing numbers of infected leaves and galls per leaf in Eucalyptus camaldulensis saplings following infection with Ophelimus maskelli in the presence of geraniol, citronellal, mineral oil control or eucalyptol control under free choice experiment setting. Bars are coded according to the number of galls per leaf. *p<0.05 compared to both mineral oil and Eucalyptol controls as determined by Dunnett's test.

FIG. 4 is a scheme of expression constructs, according to some embodiments of the invention that were transformed into Eucalyptus Urophylla (E. Urophylla)×Eucalyptus Tereticornis (E. Tereticornis) hybrids.

FIG. 5 is a bar graph demonstrating that transformation of E. Urophylla×E. Tereticornis hybrid plants with construct C (encoding GS, GR and GD, event POC-1-9A) results in a modified monoterpene profile compared to the wild-type (WT) plants. Shown are concentrations of eucalyptol, α-pinene geraniol, geranial (αcitral) and neral (βcitral) extracted from leaves of WT and transgenic plants.

FIG. 6 is a bar graph demonstrating monoterpenes profile in Eucalyptus trees expressing GS (i.e. GS transgenic lines). Three transgenic events (marked by A, B and C) were tested for monoterpenes profile by GC-MS analysis. The results are an average of 3 plants (n=3) (bars indicates SE).

FIG. 7 is a bar graph demonstrating real time PCR analysis of GS, and endogenous monoterpene expression in GS plants and WT. Gene expression is shown as a relative expression compared to housekeeping gene TEF. In each event the average of 3 plants is presented (n=3).

FIG. 8 is a schematic illustration of an asymmetric bioreduction of activated alkenes bearing an activating electron-withdrawing group (EWG) by enoate reductases. EWG pertains to ketone, aldehyde, carboxylic acid or anhydride, lactone, imide or nitro.

### DESCRIPTION OF SPECIFIC EMBODIMENTS OF THE INVENTION

The present invention, in some embodiments thereof, relates to nucleic acid constructs, plants comprising same and uses thereof in enhancing plant pest resistance and altering plant monoterpene profile.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Eucalyptus species are commercially important woody plants used for industrial purposes such as essential oil production, wood pulp, charcoal and biomass fuel. However, pest infestation is extensive and poses a real threat to natural as well as cultivated populations of eucalyptus worldwide. Efforts to control pest infection have included attempts to isolate naturally resistant plants and use of natural predators; however these efforts have met with limited or no success.

Monoterpenes, plant volatile compounds which are the main constituents of essential oils of plants, contribute to the aromatic profile of plants and play major ecological and physiological roles in flower pollination and in responses to biotic or abiotic stress. Several monoterpenes are also known to act as natural pesticides in plants and have been used as pest repellents for e.g. biting flies and for home and garden pests. The composition and expression of enzymes involved in the biosynthetic pathway of monoterpenes leads to diverse monoterpene profiles between species. Thus, for example, monoterpenes such as geraniol, geranial, neral, citronellol and/or citronellal are found in minimal or undetectable quantities in most eucalyptus species.

Whilst reducing the present invention to practice, the present inventors sought to reconstitute the monoterpene synthesis pathway in eucalyptus which would be effective in pest repelling and result in oil composition of unprecedented properties.

Thus, the present inventors have successfully heterologously expressed Geraniol synthase (GS), GS+Geraniol reductase (GR), GS+GR+Geraniol dehydrogenase (GD), GS+GD, and GS+GD+Citral reductase (CR) in eucalyptus and managed to alter the plant monoterpene profile leading to the production of monoterpenes that, as mentioned, are found in minimal or undetectable quantities in most eucalyptus species and enhances plant pest resistance.

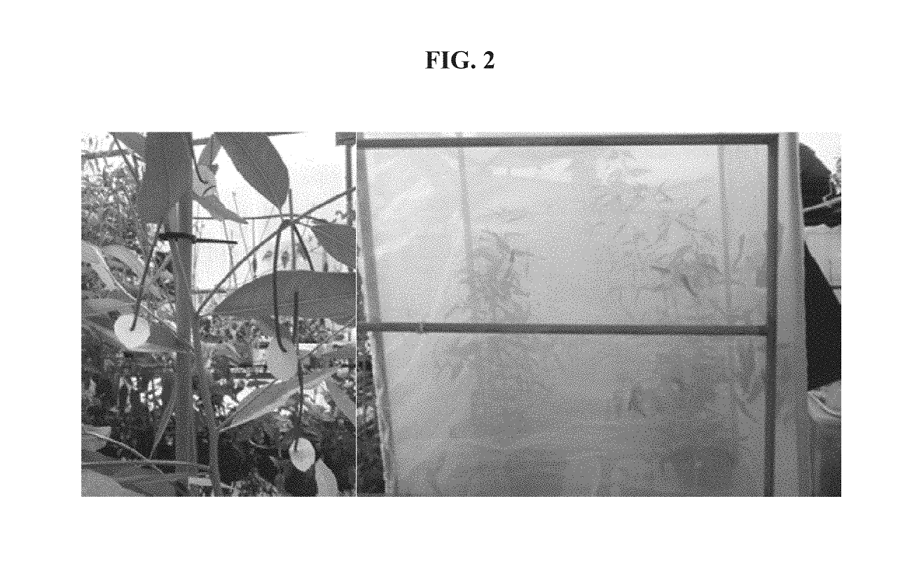

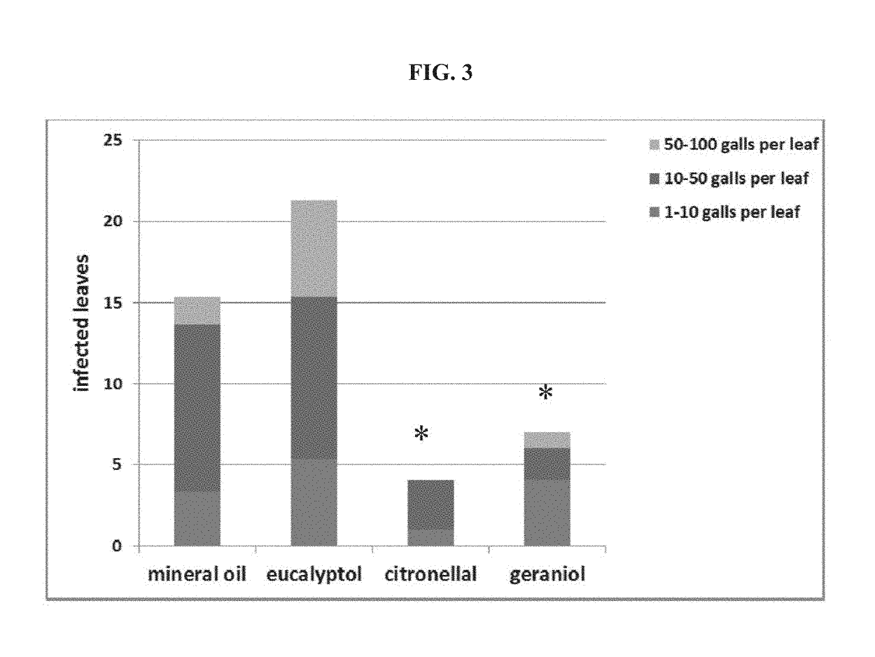

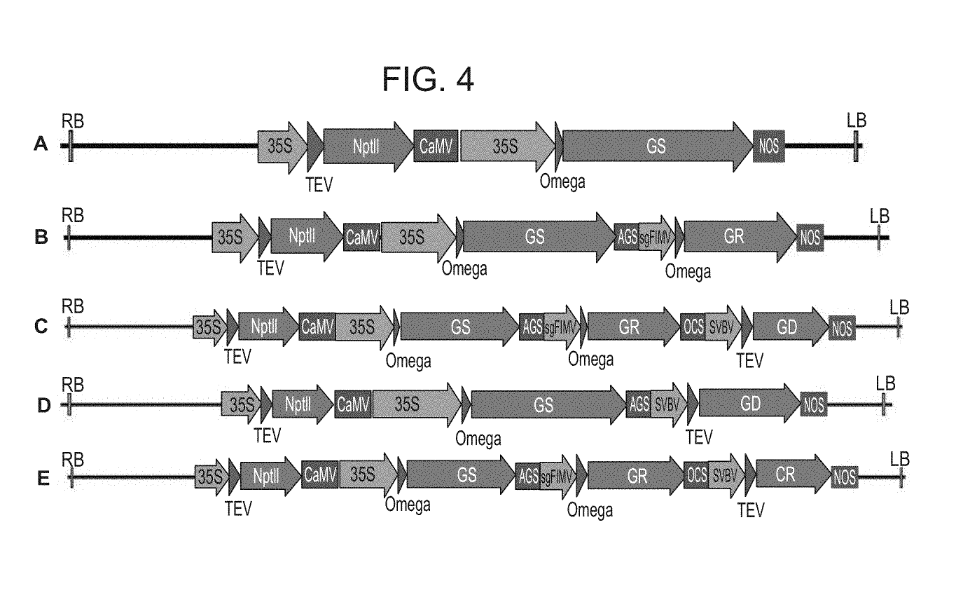

As is illustrated hereinunder and in the examples section which follows, the present inventors have shown that treatment with geraniol or citronellal significantly increased resistance of Eucalyptus camaldulensis saplings to Ophelimus maskelli infection in a free choice experiment setting (Example 1, FIGS. 2-3). Following, enzymes i.e., GS, GS+GR, GS+GR+GD, GS+GD or GS+GD+CR, expression were cloned into plant expression vectors and transformed into E. Urophylla×E. Tereticornis hybrid plants (Examples 2A-C, FIG. 4). The GR enzyme was obtained from both single cell organisms (e.g. S. cerevisiae) and from multicell organisms (e.g. plants such as from rubber tree, tomato, Arabidopsis thaliana, Rosa multiflora and Eucalyptus grandis) as exemplified in Examples 2A and 2B, respectively. Furthermore, in order to enable expression of the enzymes in the cell chloroplast, the peroxisome C-terminus tri-amino acid signal (SRL) was deleted from the amino acid sequence of the polypeptide, e.g. of Geraniol reductase (GR), and a chloroplast leader peptide was added (e.g. chloroplast CTP2, as set forth in SEQ ID NO: 91) (Example 2B).

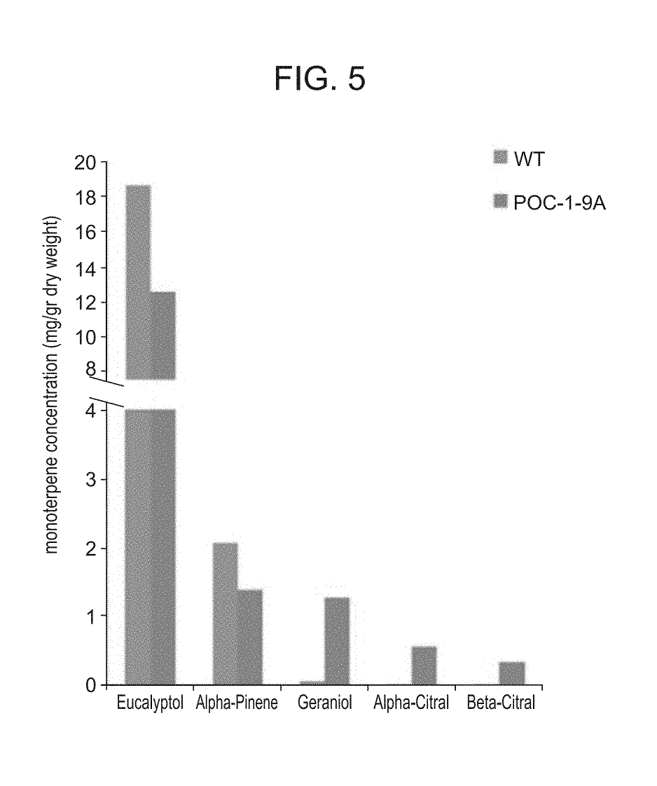

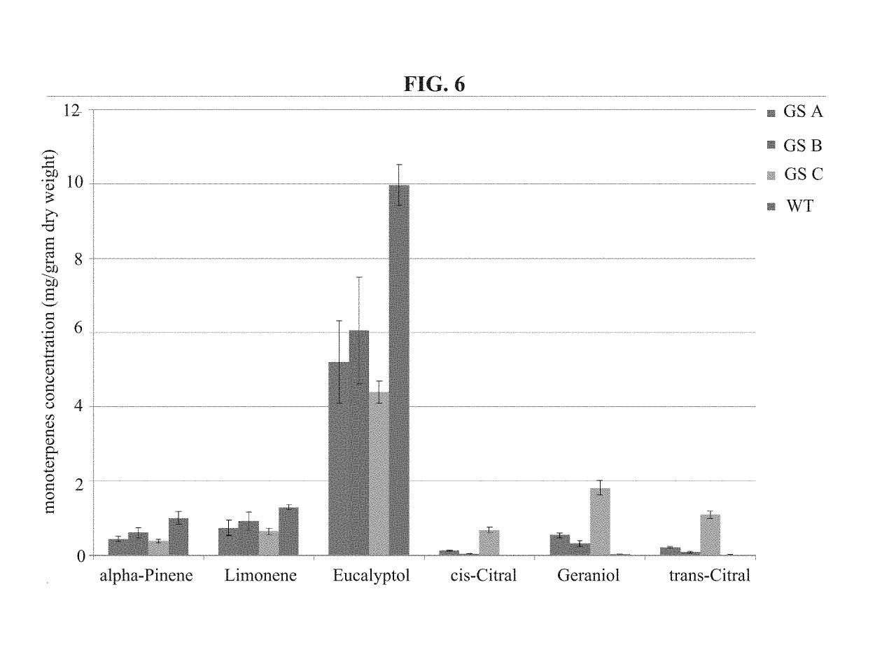

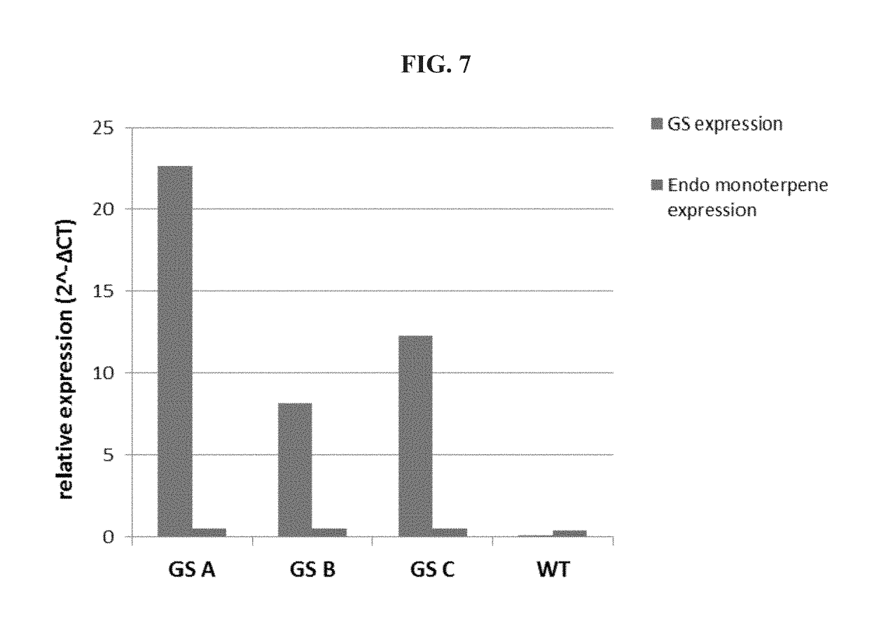

The transgenic plants exhibited modified monoterpene profile with elevated levels of monoterpenes such as geraniol, geranial and neral (Example 3, FIG. 5). Furthermore, expression of GS in Eucalyptus trees was exemplified (Example 4, FIG. 7) and resulted in a marked production of Geraniol, cis-citral and trans-citral monoterpenes (Example 4, FIG. 6). Moreover, the transgenic plants were more resistant to Glycaspis brimblecombei, Thaumastocoris peregrinus, Leptocybe invasa and Ophelimus maskelli infections (Examples 5-7).

Thus, the present teachings suggest that heterologous expression of GS, GR, GD, CR and combinations thereof can be used for conferring pest resistance. Such engineered plants can be utilized for example in the pulp industry where pest infestations of trees such as eucalyptus cause serious annual losses, as well as in other industries that rely on extensive plant cultivation. Alternatively or additionally, heterologous expression of GS, GR, GD, CR and combinations thereof can be used for altering plant monoterpene profile thereby producing oil enriched in e.g. geraniol, geranial, neral, citronellol and/or citronellal. Such oils can be utilized for example as pest repellents, as well as sources of aromas to add flavor and fragrance to foods and cosmetics.

As used herein, the term “monoterpene” refers to a compound having a 10-carbon skeleton with non-linear branches. Monoterpene compounds have two isoprene units connected in a head-to-end manner. The term “monoterpene” also refers to monoterpene derivatives and analogs also known as “monoterpenoids”. Monoterpene derivatives/analogs therefore include, but are not limited to, alcohols, ketones, aldehydes, ethers, acids, hydrocarbons without an oxygen functional group. Typically, monoterpenes are volatile compounds however; they may be further modified by conjugation to larger moieties such as sugar residues, which usually renders them non-volatile.

Non-limiting examples of monoterpenes include geraniol, nerol, neral, geranial, citral, citronellol, citronellal, limonene, pinene, terpinene, menthane, carveol, linalool, S-linalool, safrol, cinnamic acid, apiol, thymol, carvone, camphor and derivatives thereof. For information about the structure and synthesis of terpenes, see Kirk-Othmer Encyclopedia of Chemical Technology, Mark, et al., eds., 22:709-762 3d Ed (1983), the teachings of which are incorporated herein by reference in their entirety.

As used herein, the term “geraniol” also known as lemonol, geranyl alcohol, trans-geraniol, (E)-geraniol, refers to a monoterpene with a formula of C₁₀H₁₈O, CAS No. 106-24-1, IUPAC name (trans)-3,7-Dimethyl-2,6-octadien-1-ol.

As used herein, the term “geranial” refers to the E isomer of citral, also known as citral A.

As used herein, the term “neral” refers to the Z isomer of citral, also known as citral B.

As used herein the term “citral” refers to a monoterpene with a molecular formula of C₁₀H₁₆O, CAS No. 5392-40-5, IUPAC name 3,7-dimethylocta-2,6-dienal. According to one embodiment, citral is alpha citral (also known as geranial) or beta citral (also known as neral).

As used herein, the term “citronellol” refers to a monoterpene with a molecular formula of C₁₀H₂₀O, CAS No. 106-22-9, IUPAC name 3,7-Dimethyloct-6-en-1-ol.

As used herein, the term “citronellal” also known as rhodinal, 3,7-Dimethyl-6-octenal, 6-Octenal, 3,7-dimethyl-, 2,3-Dihydrocitral and citronellel, refers to a monoterpene with a molecular formula of C10H₁₈O, CAS No. 106-23-0, IUPAC name 3,7-dimethyloct-6-en-1-al.

The composition and expression of the enzymes involved in the biosynthetic pathway of monoterpenes leads to diverse monoterpene profiles between species.

Thus, according to an aspect of the present invention there are provided nucleic acid constructs which can be used in the transformation of cells, e.g. plant cells, e.g. woody plant.

According to an embodiment, the nucleic acid construct comprises a nucleic acid sequence encoding at least one polypeptide selected from the group consisting of Geraniol Synthase (GS), Geraniol Reductase (GR), Geraniol Dehydrogenase (GD) and Citral Reductase (CR) and a cis-acting regulatory element for directing expression of the nucleic acid sequence such as in plant cells.

According to a specific embodiment, the nucleic acid construct comprises a nucleic acid sequence encoding GD and a cis-acting regulatory element for directing expression of the nucleic acid sequence such as in a plant cell.

According to a specific embodiment, the nucleic acid construct comprises a nucleic acid sequence encoding CR and a cis-acting regulatory element for directing expression of the nucleic acid sequence in a plant cell. According to a specific embodiment, the nucleic acid sequence further comprises a chloroplast leader peptide.

According to another specific embodiment, the nucleic acid construct comprises a nucleic acid sequence encoding GR and a cis-acting regulatory element for directing expression of the nucleic acid sequence in a plant cell, wherein the nucleic acid sequence is devoid of a peroxisome C-terminus tri-amino acid signal (SRL). According to a specific embodiment, the nucleic acid sequence further comprises a chloroplast leader peptide.

According to one embodiment, the nucleic acid sequence encoding an enzyme is derived from the Plantae kingdom. Accordingly the nucleic acid sequence encoding GS, GR, GD and/or CR may be derived from a plant.

According to specific embodiment, the nucleic acid construct comprises a nucleic acid sequence encoding GR and a cis-acting regulatory element for directing expression of the nucleic acid sequence in a plant cell, wherein the nucleic acid sequence is derived from the Plantae kingdom and is devoid of a peroxisome C-terminus tri-amino acid signal (SRL). According to a specific embodiment, the nucleic acid sequence further comprises a chloroplast leader peptide.

According to yet another embodiment, the nucleic acid construct comprises a nucleic acid sequence encoding at least two polypeptides selected from the group consisting of GS, GR, GD and CR and at least one cis-acting regulatory element for directing expression of the nucleic acid sequence such as in a plant cell.

According to yet another embodiment, the nucleic acid construct comprises a nucleic acid sequence encoding GS, GR and GD and at least one cis-acting regulatory element for directing expression of the nucleic acid sequence.

According to yet another embodiment, the nucleic acid construct comprises a nucleic acid sequence encoding GS, GR, GD and CR and at least one cis-acting regulatory element for directing expression of the nucleic acid sequence.

According to a specific embodiment, the nucleic acid construct comprises a nucleic acid sequence encoding GS+GR and at least one cis-acting regulatory element for directing expression of the nucleic acid sequence.

According to a specific embodiment, the nucleic acid construct comprises a nucleic acid sequence encoding GD+GR and at least one cis-acting regulatory element for directing expression of the nucleic acid sequence.

According to a specific embodiment, the nucleic acid construct comprises a nucleic acid sequence encoding GS+GD and at least one cis-acting regulatory element for directing expression of the nucleic acid sequence.

According to a specific embodiment, the nucleic acid construct comprises a nucleic acid sequence encoding GS+CR and at least one cis-acting regulatory element for directing expression of the nucleic acid sequence.

According to a specific embodiment, the nucleic acid construct comprises a nucleic acid sequence encoding GR+CR and at least one cis-acting regulatory element for directing expression of the nucleic acid sequence.

According to a specific embodiment, the nucleic acid construct comprises a nucleic acid sequence encoding GD+CR and at least one cis-acting regulatory element for directing expression of the nucleic acid sequence.

According to a specific embodiment, the nucleic acid construct comprises a nucleic acid sequence encoding GS+GR+GD and at least one cis-acting regulatory element for directing expression of the nucleic acid sequence such as in a plant cell.

According to a specific embodiment, the nucleic acid construct comprises a nucleic acid sequence encoding GS+GR+CR and at least one cis-acting regulatory element for directing expression of the nucleic acid sequence such as in a plant cell.

According to a specific embodiment, the nucleic acid construct comprises a nucleic acid sequence encoding GS+GD+CR and at least one cis-acting regulatory element for directing expression of the nucleic acid sequence such as in a plant cell.

According to a specific embodiment, the nucleic acid construct comprises a nucleic acid sequence encoding GR+GD+CR and at least one cis-acting regulatory element for directing expression of the nucleic acid sequence such as in a plant cell.

According to a specific embodiment, the nucleic acid construct comprises a nucleic acid sequence encoding GS+GR+GD+CR and at least one cis-acting regulatory element for directing expression of the nucleic acid sequence such as in a plant cell.

According to another aspect of the present invention there is provided a nucleic acid construct system comprising at least two nucleic acid constructs expressing at least two polypeptides selected from the group consisting of Geraniol Synthase (GS), Geraniol Reductase (GR), Geraniol Dehydrogenase (GD) and Citral Reductase (CR) and at least one cis-acting regulatory element for directing expression of the at least two polypeptides such as in plant cells.

According to a specific embodiment, the nucleic acid construct system comprises at least two nucleic acid constructs expressing Geraniol Synthase (GS), and Geraniol Reductase (GR) each of the GS and GR being operably linked to a cis-acting regulatory element for directing expression of the at least two polypeptides in plant cells.

According to a specific embodiment, the nucleic acid construct system comprises at least two nucleic acid constructs expressing Geraniol Synthase (GS), and Geraniol Dehydrogenase (GD) each of the GS and GD being operably linked to a cis-acting regulatory element for directing expression of the at least two polypeptides in plant cells.

According to a specific embodiment, the nucleic acid construct system comprises at least two nucleic acid constructs expressing Geraniol Reductase (GR) and Geraniol Dehydrogenase (GD) each of the GR and GD being operably linked to a cis-acting regulatory element for directing expression of the at least two polypeptides in plant cells.

According to a specific embodiment, the nucleic acid construct system comprises at least two nucleic acid constructs expressing Geraniol Synthase (GS), and Citral Reductase (CR) each of the GS and CR being operably linked to a cis-acting regulatory element for directing expression of the at least two polypeptides in plant cells.

According to a specific embodiment, the nucleic acid construct system comprises at least two nucleic acid constructs expressing Geraniol Reductase (GR) and Citral Reductase (CR) each of the GR and CR being operably linked to a cis-acting regulatory element for directing expression of the at least two polypeptides in plant cells.

According to a specific embodiment, the nucleic acid construct system comprises at least two nucleic acid constructs expressing Geraniol Dehydrogenase (GD) and Citral Reductase (CR) each of the GD and CR being operably linked to a cis-acting regulatory element for directing expression of the at least two polypeptides in plant cells.

According to a specific embodiment, the nucleic acid construct system comprises at least two (e.g. three) nucleic acid constructs expressing Geraniol Synthase (GS), Geraniol Reductase (GR) and/or Geraniol Dehydrogenase (GD) each of the GS, GR and GD being operably linked to a cis-acting regulatory element for directing expression of the at least two polypeptides in plant cells.

According to a specific embodiment, the nucleic acid construct system comprises at least two (e.g. three) nucleic acid constructs expressing Geraniol Synthase (GS), Geraniol Reductase (GR) and/or Citral Reductase (CR) each of the GS, GR and CR being operably linked to a cis-acting regulatory element for directing expression of the at least two polypeptides in plant cells.

According to a specific embodiment, the nucleic acid construct system comprises at least two (e.g. three) nucleic acid constructs expressing Geraniol Synthase (GS), Geraniol Dehydrogenase (GD) and/or Citral Reductase (CR) each of the GS, GD and CR being operably linked to a cis-acting regulatory element for directing expression of the at least two polypeptides in plant cells.

According to a specific embodiment, the nucleic acid construct system comprises at least two (e.g. three) nucleic acid constructs expressing Geraniol Reductase (GR), Geraniol Dehydrogenase (GD) and/or Citral Reductase (CR) each of the GR, GD and CR being operably linked to a cis-acting regulatory element for directing expression of the at least two polypeptides in plant cells.

According to a specific embodiment, the nucleic acid construct system comprises at least two (e.g. three, four) nucleic acid constructs expressing Geraniol Synthase (GS), Geraniol Reductase (GR), Geraniol Dehydrogenase (GD) and/or Citral Reductase (CR) each of the GS, GR, GD and CR being operably linked to a cis-acting regulatory element for directing expression of the at least two polypeptides in plant cells.

As mentioned hereinabove, the present inventors have shown that the constructs described herein comprising nucleic acid sequences encoding enzymes may catalyze production of specific monoterpenes even in plants in which this synthetic pathway is seemingly silenced or absent.

As used herein, “Geraniol Synthase” (GS) E.C. 3.1.7.11. also known as Geranyl diphosphate diphosphatase, geranyl pyrophosphate pyrophosphatase, GES and CtGES, refers to a functional GS and fragments thereof which catalyze the production of geraniol from Geranyl diphosphate (GDP). According to specific embodiments, GS is the Ocimum Basilicum (basil) GS such as provided in NCBI accession number AAR11765 (SEQ ID NO: 1) and NCBI gi|75224312 (SEQ ID NO: 2) and gi|75251480 (SEQ ID NO: 3). According to specific embodiments, GS is the Olea europaea GS such as provided in NCBI gi|406780549 (SEQ ID NO: 4), gi|406780547 (SEQ ID NO: 5) and gi|385211784 (SEQ ID NO: 6). According to specific embodiments, GS is the Phyla dulcis GS such as provided in NCBI gi|301131134 (SEQ ID NO: 7). According to other specific embodiments, GS is the Catharanthus roseus GS such as provided in NCBI gi|380513810 (SEQ ID NO: 8). According to other specific embodiments, GS is the Picrorhiza kurroa GS such as provided in NCBI gi|618884964 (SEQ ID NO: 9). According to other specific embodiments, GS is the Valeriana officinalis GS such as provided in NCBI gi|569344778 (SEQ ID NO: 10). According to other specific embodiments, GS is the Vitis vinifera GS such as provided in NCBI gi|313755452 (SEQ ID NO: 11) and gi|526118024 (SEQ ID NO: 12).

As used herein, “Geraniol Reducatase” (GR) E.C. 1.6.99.1 also known as Old Yellow Enzyme 2, OYE2, NADPH dehydrogenase, enone reductase 2 and ERED 2, refers to a functional GR and fragments thereof able to catalyze the production of citronellol from geraniol. According to specific embodiments, GR is the S. cerevisiae GR such as provided in NCBI accession number Q03558 (SEQ ID NO: 13) and NCBI gi|6321973 (SEQ ID NO: 14), gi|151944124 (SEQ ID NO: 15), gi|45270462 (SEQ ID NO: 16), gi|323308791 (SEQ ID NO: 17), gi|584370807 (SEQ ID NO: 18), gi|349578731 (SEQ ID NO: 19), gi|584476449 (SEQ ID NO: 20) and gi|347803140 (SEQ ID NO: 21). According to specific embodiments, GR is the Candida sake GR such as provided in NCBI gi|347803126 (SEQ ID NO: 22). According to other specific embodiments, GR is the Kazachstania exigua GR such as provided in NCBI gi|347803128 (SEQ ID NO: 23). According to other specific embodiments, GR is the Naumovozyma castellii GR such as provided in NCBI gi|347803138 (SEQ ID NO: 24). According to other specific embodiments, GR is the Kazachstania spencerorum GR such as provided in NCBI gi|347803134 (SEQ ID NO: 25). According to other specific embodiments, GR is the Kazachstania solicola GR such as provided in NCBI gi|347803132 (SEQ ID NO: 26). According to other specific embodiments, GR is the Nakaseomyces bacillisporus GR such as provided in NCBI gi|347803136 (SEQ ID NO: 27). According to other specific embodiments, GR is the Saccharomyces cerevisiae×Saccharomyces kudriavzevii GR such as provided in NCBI gi|365760283 (SEQ ID NO: 28). According to other specific embodiments, GR is the kudriavzevii GR such as provided in NCBI gi|401841420 (SEQ ID NO: 29). According to other specific embodiments, GR is 12-Oxophytodienoate Reductase from Hevea brasiliensis (rubber tree) such as provided in UniProt Q4ZJ73 (SEQ ID NO: 81). According to other specific embodiments, GR is from Arabidopsis thaliana such as provided in UniProt Q9FUP0(SEQ ID NO: 83). According to other specific embodiments, GR is from Solanum lycopersicum (Tomato) such as provided in UniProt Q9FEW9 (SEQ ID NO: 85). According to other specific embodiments, GR is from Eucalyptus grandis such as provided in UniProt A0A059CML0 (SEQ ID NO: 87). According to other specific embodiments, GR is from Rosa multiflora such as provided in Transcriptom (doi: 10.3389/fpls.2015.00249) (SEQ ID NO: 89).

As used herein, “Geraniol Dehydrogenase” (GD) EC No. 1.1.1.183 also known as Citronellol dehydrogenase, nerol dehydrogenase and GEDH1 refers to a functional GD and fragments thereof able to catalyze the production of geranial from geraniol and citronellal from citronellol. According to specific embodiments, GD is the basil GD such as provided in NCBI accession number Q2KNL6 (SEQ ID NO: 30) and NCBI gi|122200955 (SEQ ID NO: 31) and gi|389889215 (SEQ ID NO: 32). According to specific embodiments, GD is the Perilla setoyensis GS such as provided in NCBI gi|427776200 (SEQ ID NO: 33). According to other specific embodiments, GD is the Perilla citriodora GS such as provided in NCBI gi|427776198 (SEQ ID NO: 34). According to other specific embodiments, GD is the Perilla frutescens GD such as provided in NCBI gi|427776196 (SEQ ID NO: 35).

As used herein, “Citral Reductase” (CR) also known as putative oxidoreductase or enoate reductase refers to a functional CR and fragments thereof able to catalyze the production of citronellal from citral. According to specific embodiments, CR is from Gluconobacter oxydans such as provided in Q5FTL6 (SEQ ID NO: 79).

According to a specific embodiment, GS nucleic acid sequence comprises a nucleic acid sequence selected from the group consisting of SEQ ID NOs: 36-46 or a nucleic acid sequence which is at least 50%, at least 60%, at least 70%, at least 80%, at least 85%; at least 90%, at least 95%, at least 98%, at least 99% or 100% identical or homologous to SEQ ID NOs: 36-46 and catalyzing the production of geraniol from Geranyl diphosphate (GDP) (also referred to as a functional homolog).

According to a specific embodiment GS amino acid sequence comprises an amino acid sequence selected from the group consisting of SEQ ID NOs: 1-12 or an amino acid sequence which is at least 50%, at least 60%, at least 70%, at least 80%, at least 85%; at least 90%, at least 95%, at least 98%, at least 99% or 100% identical or homologous to SEQ ID NOs: 1-12 and catalyzing the production of geraniol from Geranyl diphosphate (GDP).

According to specific embodiments GR nucleic acid sequence comprises a nucleic acid sequence selected from the group consisting of SEQ ID NOs: 47-57, 82, 84, 86, 88 and 90, or a nucleic acid sequence which is at least 60%, at least 70%, at least 80%, at least 85%; at least 90%, at least 95%, at least 98%, at least 99% or 100% identical or homologous to SEQ ID NOs: 47-57, 82, 84, 86, 88 or 90, and catalyzing the production of citronellol from geraniol (also referred to as a functional homolog).

According to specific embodiments GR amino acid sequence comprises an amino acid sequence selected from the group consisting of SEQ ID NOs: 13-29, 81, 83, 85, 87 and 89, or an amino acid sequence which is at least 60%, at least 70%, at least 80%, at least 85%; at least 90%, at least 95%, at least 98%, at least 99% or 100% identical or homologous to SEQ ID NOs: 13-29, 81, 83, 85, 87 or 89, and catalyzing the production of citronellol from geraniol.

According to specific embodiments GD nucleic acid sequence comprises a nucleic acid sequence selected from the group consisting of SEQ ID NOs: 58-63 or a nucleic acid sequence which is at least 60%, at least 70%, at least 80%, at least 85%; at least 90%, at least 95%, at least 98%, at least 99% or 100% identical or homologous to SEQ ID NOs: 58-63 and catalyzing the production of geranial from geraniol and citronellal from citronellol (also referred to as a functional homolog).

According to specific embodiments GD amino acid sequence comprises an amino acid sequence selected from the group consisting of SEQ ID NO: 30-35 or an amino acid sequence which is at least 60%, at least 70%, at least 80%, at least 85%; at least 90%, at least 95%, at least 98%, at least 99% or 100% identical or homologous to SEQ ID NOs: 30-35 and catalyzing the production of geranial from geraniol and citronellal from citronellol.

According to specific embodiments CR nucleic acid sequence comprises a nucleic acid sequence as set forth in SEQ ID NOs: 80 or a nucleic acid sequence which is at least 60%, at least 70%, at least 80%, at least 85%; at least 90%, at least 95%, at least 98%, at least 99% or 100% identical or homologous to SEQ ID NO: 80 and catalyzing the production of citronellal from citral (also referred to as a functional homolog).

According to specific embodiments CR amino acid sequence comprises an amino acid sequence as set forth in SEQ ID NO: 79 or an amino acid sequence which is at least 60%, at least 70%, at least 80%, at least 85%; at least 90%, at least 95%, at least 98%, at least 99% or 100% identical or homologous to SEQ ID NO: 79 and catalyzing the production of citronellal from citral.

Sequence identity or homology can be determined using any protein or nucleic acid sequence alignment algorithm such as Blast, ClustalW, and MUSCLE, such as using default parameters.

The terms “polypeptide” and “protein” are interchangeably used.

As used herein the term “at least one polypeptide” refers to one, two, three, or all four polypeptides i.e.: GS; GR; GD; CR; GS+GR; GS+GD; GS+CR; GR+GD; GR+CR; GD+CR; GS+GR+GD, GS+GR+CR, GS+GD+CR, GR+GD+CR, or GS+GR+GD+CR.

According to a specific embodiment the at least one polypeptide comprises GS.

According to a specific embodiment the at least one polypeptide comprises GR.

According to a specific embodiment the at least one polypeptide comprises GD.

According to a specific embodiment the at least one polypeptide comprises CR. According to another specific embodiment the at least one polypeptide comprises at least two polypeptides.

As used herein, the term “at least two polypeptides” refers to two, three or all four polypeptides i.e.: GS+GR; GS+GD; GS+CR; GR+GD; GR+CR; GD+CR; GS+GR+GD, GS+GR+CR, GS+GD+CR, GR+GD+CR, or GS+GR+GD+CR.

According to a specific embodiment the at least two polypeptides comprise GS and GR.

According to a specific embodiment the at least two polypeptides comprise GS and GD.

According to a specific embodiment the at least two polypeptides comprise GS and CR.

According to a specific embodiment the at least two polypeptides comprise GR and GD.

According to a specific embodiment the at least two polypeptides comprise GR and CR.

According to a specific embodiment the at least two polypeptides comprise GD and CR.

According to another specific embodiment the at least two polypeptides comprise three polypeptides, i.e. GS, GR and GD.

According to another specific embodiment the at least two polypeptides comprise three polypeptides, i.e. GS, GR and CR. According to another specific embodiment the at least two polypeptides comprise three polypeptides, i.e. GS, GD and CR.

According to another specific embodiment the at least two polypeptides comprise three polypeptides, i.e. GR, GD and CR.

According to another specific embodiment the at least two polypeptides comprise four polypeptides, i.e. GS, GR, GD and CR.

As used herein, the terms “polynucleotide” and “nucleic acid sequence” which are interchangeably used, refer to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements, as described in further detail below.

Nucleic acid sequences of the polypeptides of some embodiments of the invention may be optimized for plant expression. Examples of such sequence modifications include, but are not limited to, an altered G/C content to more closely approach that typically found in the plant species of interest, and the removal of codons atypically found in the plant species commonly referred to as codon optimization.

The phrase “codon optimization” refers to the selection of appropriate DNA nucleotides for use within a structural gene or fragment thereof that approaches codon usage within the plant of interest. Therefore, an optimized gene or nucleic acid sequence refers to a gene in which the nucleotide sequence of a native or naturally occurring gene has been modified in order to utilize statistically-preferred or statistically-favored codons within the plant. The nucleotide sequence typically is examined at the DNA level and the coding region optimized for expression in the plant species determined using any suitable procedure, for example as described in Sardana et al. (1996, Plant Cell Reports 15:677-681). In this method, the standard deviation of codon usage, a measure of codon usage bias, may be calculated by first finding the squared proportional deviation of usage of each codon of the native gene relative to that of highly expressed plant genes, followed by a calculation of the average squared deviation. The formula used is: 1SDCU=n=1N[(Xn−Yn)/Yn]2/N, where Xn refers to the frequency of usage of codon n in highly expressed plant genes, where Yn to the frequency of usage of codon n in the gene of interest and N refers to the total number of codons in the gene of interest. A table of codon usage from highly expressed genes of dicotyledonous plants is compiled using the data of Murray et al. (1989, Nuc Acids Res. 17:477-498).

One method of optimizing the nucleic acid sequence in accordance with the preferred codon usage for a particular plant cell type is based on the direct use, without performing any extra statistical calculations, of codon optimization tables such as those provided on-line at the Codon Usage Database through the NIAS (National Institute of Agrobiological Sciences) DNA bank in Japan (www(dot)kazusa(dot)or(dot)jp/codon/). The Codon Usage Database contains codon usage tables for a number of different species, with each codon usage table having been statistically determined based on the data present in Genbank.

By using the above tables to determine the most preferred or most favored codons for each amino acid in a particular species (for example, eucalyptus), a naturally-occurring nucleotide sequence encoding a protein of interest can be codon optimized for that particular plant species. This is effected by replacing codons that may have a low statistical incidence in the particular species genome with corresponding codons, in regard to an amino acid, that are statistically more favored.

However, one or more less-favored codons may be selected to delete existing restriction sites, to create new ones at potentially useful junctions (5′ and 3′ ends to add signal peptide or termination cassettes, internal sites that might be used to cut and splice segments together to produce a correct full-length sequence), or to eliminate nucleotide sequences that may negatively affect mRNA stability or expression.

The naturally-occurring encoding nucleotide sequence may already, in advance of any modification, contain a number of codons that correspond to a statistically-favored codon in a particular plant species. Therefore, codon optimization of the native nucleotide sequence may comprise determining which codons, within the native nucleotide sequence, are not statistically-favored with regards to a particular plant, and modifying these codons in accordance with a codon usage table of the particular plant to produce a codon optimized derivative. A modified nucleotide sequence may be fully or partially optimized for plant codon usage provided that the protein encoded by the modified nucleotide sequence is produced at a level higher than the protein encoded by the corresponding naturally occurring or native gene. Construction of synthetic genes by altering the codon usage is described in for example PCT Patent Application 93/07278.

Thus, embodiments of the invention encompass nucleic acid sequences described hereinabove; fragments thereof, sequences hybridizable therewith, sequences homologous thereto, sequences orthologous thereto, sequences encoding similar polypeptides with different codon usage, altered sequences characterized by mutations, such as deletion, insertion or substitution of one or more nucleotides, either naturally occurring or man induced, either randomly or in a targeted fashion.

Constructs useful in the methods according to the present invention may be constructed using recombinant DNA technology well known to persons skilled in the art. The gene constructs may be inserted into vectors, which may be commercially available, suitable for transforming into e.g. plants and suitable for expression of the gene of interest in the transformed cells. The genetic construct can be an expression vector whereby, as mentioned, the heterologous nucleic acid sequence is operably linked to a cis-acting regulatory element allowing expression in the cells, such as in plant cells.

As used herein, the phrase “cis acting regulatory element” refers to a polynucleotide sequence, preferably a promoter, which binds a trans acting regulator and regulates the transcription of a coding sequence located downstream thereto.

According to specific embodiments the cis-acting regulatory element comprises a promoter sequence.

As used herein, the phrase “operably linked” refers to a functional positioning of the cis-regulatory element (e.g., promoter) so as to allow regulating expression of the selected nucleic acid sequence. For example, a promoter sequence may be located upstream of the selected nucleic acid sequence in terms of the direction of transcription and translation.

According to an embodiment, the promoter in the nucleic acid construct of the present invention is a plant promoter which serves for directing expression of the heterologous nucleic acid molecule within plant cells.

As used herein the phrase “plant promoter” refers to a promoter sequence, including any additional regulatory elements added thereto or contained therein, is at least capable of inducing, conferring, activating or enhancing expression in a plant cell, tissue or organ, preferably a woody plant cell, tissue, or organ. Such a promoter can be derived from a plant, bacterial, viral, fungal or animal origin. Such a promoter can be constitutive, i.e., capable of directing high level of gene expression in a plurality of plant tissues, tissue specific, i.e., capable of directing gene expression in a particular plant tissue or tissues, inducible, i.e., capable of directing gene expression under a stimulus, or chimeric, i.e., formed of portions of at least two different promoters.

According to specific embodiments the promoter is a constitutive promoter.

Examples of constitutive plant promoters include, without being limited to, CaMV35S and CaMV19S promoters, Figwort mosaic virus subgenomic transcript (sgFiMV) promoter, Strawberry vein banding virus (SVBV) promoter, FMV34S promoter, sugarcane bacilliform badnavirus promoter, CsVMV promoter, Arabidopsis ACT2/ACT8 actin promoter, Arabidopsis ubiquitin UBQ1 promoter, barley leaf thionin BTH6 promoter, and rice actin promoter.

According to a specific embodiment the constitutive promoter is selected from the group consisting of Cauliflower mosaic virus (CaMV) 35S promoter, Figwort mosaic virus subgenomic transcript (sgFiMV) promoter and Strawberry vein banding virus (SVBV) promoter.

According to specific embodiments the promoter is a tissue specific promoter. Non-limiting examples of tissue specific promoters include those described in Yamamoto et al. (1997) Plant J. 12(2):255-265; Kawamata et al. (1997) Plant Cell Physiol. 38(7):792-803; Hansen et al. (1997) Mol. Gen. Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol. Biol. 23(6):1129-1138; Matsuoka et al. (1993) Proc. Natl. Acad. Sci. U.S.A. 90(20):9586-9590; and Guevara-Garcia et al. (1993) Plant J. 4(3):495-505.

Other exemplary promoters useful for the methods of some embodiments of the invention are presented in Tables I, II and III.

TABLE I
Exemplary constitutive promoters for use in the performance of some
embodiments of the invention

| Gene Source | Expression Pattern | Reference |
|---|---|---|
| Actin | constitutive | McElroy et al., Plant Cell, |
| | | 2: 163-171, 1990 |
| CAMV 35S | constitutive | Odell et al., Nature, 313: |
| | | 810-812, 1985 |
| CaMV 19S | constitutive | Nilsson et al., Physiol. |
| | | Plant 100: 456-462, 1997 |
| GOS2 | constitutive | de Pater et al., Plant J |
| | | Nov; 2(6): 837-44, 1992 |
| ubiquitin | constitutive | Christensen et al., Plant |
| | | Mol. Biol. 18: 675-689, |
| | | 1992 |
| Rice cyclophilin | constitutive | Bucholz et al., Plant Mol |
| | | Biol. 25(5): 837-43, 1994 |
| Maize H3 histone | constitutive | Lepetit et al., Mol. Gen. |
| | | Genet. 231: 276-285, |
| | | 1992 |
| Actin 2 | constitutive | An et al., Plant J. |
| | | 10(1); 107-121, 1996 |

TABLE II
Exemplary seed-preferred promoters for use in the performance of some
embodiments of the invention

| Gene Source | Expression Pattern | Reference |
|---|---|---|
| Seed specific genes | seed | Simon, et al., Plant Mol. |
| | | Biol. 5. 191, 1985; |
| | | Scofield, |
| | | et al., J. Biol. Chem. |
| | | 262: 12202, 1987.; |
| | | Baszczynski, et al., Plant |
| | | Mol. Biol. 14: 633, 1990. |
| Brazil Nut albumin | seed | Pearson' et al., Plant Mol. |
| | | Biol. 18: 235-245, 1992. |
| legumin | seed | Ellis, et al. Plant Mol. |
| | | Biol. 10: 203-214, 1988 |
| Glutelin (rice) | seed | Takaiwa, et al., Mol. Gen. |
| | | Genet. 208: 15-22, 1986; |
| | | Takaiwa, et al., FEBS |
| | | Letts. 221: 43-47, 1987 |
| Zein | seed | Matzke et al. Plant Mol |
| | | Biol, 143). 323-32 1990 |
| napA | seed | Stalberg, et al., Planta |
| | | 199: 515-519, 1996 |
| wheat LMW and HMW, | endosperm | Mol Gen Genet 216: 81- |
| glutenin-1 | | 90, 1989; NAR 17: 461-2, |
| Wheat SPA | seed | Albani et al., Plant Cell, |
| | | 9: 171-184, 1997 |
| wheat a, b and g gliadins | endosperm | EMBO3: 1409-15, 1984 |
| Barley ltrl promoter | endosperm | |
| barley B1, C, D hordein | endosperm | Theor Appl Gen 98: 1253- |
| | | 62, 1999; Plant J 4: 343- |
| | | 55, 1993; Mol Gen Genet |
| | | 250: 750-60, 1996 |
| Barley DOF | endosperm | Mena et al., The Plant |
| | | Journal, 116(1): 53-62, |
| | | 1998 |
| Biz2 | endosperm | EP99106056.7 |
| Synthetic promoter | endosperm | Vicente-Carbajosa et al., |
| | | Plant J. 13: 629-640, 1998 |
| rice prolamin NRP33 | endosperm | Wu et al., Plant Cell |
| | | Physiology 39(8) 885- |
| | | 889, 1998 |
| rice-globulin Glb-1 | endosperm | Wu et al, Plant Cell |
| | | Physiology 398) 885-889, |
| | | 1998 |
| rice OSH1 | embryo | Sato et al., Proc. Nati. |
| | | Acad. Sci. USA, 93: |
| | | 8117-8122 |
| rice alpha-globulin | endosperm | Nakase et al. Plant Mol. |
| REB/OHP-1 | | Biol. 33: 513-S22, 1997 |
| rice ADP-glucose PP | endosperm | Trans Res 6: 157-68, 1997 |
| maize ESR gene family | endosperm | Plant J 12: 235-46, 1997 |
| sorgum gamma-kafirin | endosperm | PMB 32: 1029-35, 1996 |
| KNOX | embryo | Postma-Haarsma et al., |
| | | Plant Mol. Biol. 39: 257- |
| | | 71, 1999 |
| rice oleosin | Embryo and aleuton | Wu et at., J. Biochem., |
| | | 123: 386, 1998 |
| sunflower oleosin | Seed (embryo and dry | Cummins, et al., Plant |
| | seed) | Mol. Biol. 19: 873-876, |
| | | 1992 |

TABLE III
Exemplary flower-specific promoters for use in
the performance of the invention

| | Expression | |
|---|---|---|
| Gene Source | Pattern | Reference |
| AtPRP4 | flowers | salus(dot) |
| | | medium(dot)edu/m |
| | | mg/tierney/html |
| chalene synthase | flowers | Van der Meer, et al., Plant |
| (chsA) | | Mol. Biol. 15, 95- |
| | | 109, 1990. |
| LAT52 | anther | Twell et al. Mol. Gen |
| | | Genet. 217: 240-245 |
| | | (1989) |
| apetala-3 | flowers | |

According to other specific embodiments the promoter is a chemically regulated promoter. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemically regulated promoters of interest include steroid-responsive promoters and tetracycline-inducible and tetracycline-repressible promoters (see, for example, Schena et al. (1991) Proc. Natl. Acad. Sci. USA 88:10421-10425 and Gatz et al. (1991) Mol. Gen. Genet. 227:229-237).

According to other specific embodiments the promoter is a pest-inducible promoter. These promoters direct the expression of genes in plants following infection with a pest such as bacteria, fungi, viruses, nematodes and insects. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol 89:245-254; Uknes et al. (1992) Plant Cell 4:645-656; and Van Loon (1985) Plant Mol. Virol. 4:111-116.

The nucleic acid construct (also referred to herein as an “expression vector” “expression construct” or a “vector”) of some embodiments of the invention includes additional sequences which render this vector suitable for replication and integration in prokaryotes, eukaryotes, or preferably both (e.g., shuttle vectors). In addition, typical vectors may also contain a one or more additional regulatory elements, such as transcription and translation initiation sequence, transcription and translation terminator, a 5′ leader and/or intron for enhancing transcription, a 3′-untranslated region (e.g., a sequence containing a polyadenylation signal), and a nucleic acid sequence encoding a transit or signal peptide (e.g., a chloroplast transit or signaling peptide). The expression vector of some embodiments of the invention can also include sequences engineered to enhance stability, production, purification, yield or toxicity of the expressed polypeptide.

According to specific embodiments, the nucleic acid construct of the present invention may comprise a translation enhancer such as omega translation enhancer. Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. Non-limiting examples of enhancers include the 5′-untranslated region (5′-UTR) of RNA of tobacco mosaic virus (TMV), called omega sequence, the tobacco etch virus translational enhancer and the SV40 early gene enhancer. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

The gene termination sequence, which is located 3′ to the polynucleotide to be transcribed, may be from the same gene as the gene promoter sequence or may be from a different gene. Many gene termination sequences known in the art may be usefully employed in the present invention, such as but not limited to the nopaline synthase (NOS) terminator (SEQ ID NO: 64), the CaMV terminator (SEQ ID NO: 65), the agropine synthase (AGS) terminator (SEQ ID NO: 66) and the octapin synthase (OCS) terminator (SEQ ID NO: 67).

In native tissues which synthesize monoterpenes, GS, GR and GD are targeted to plastids via plastid leader peptides. The targeting sequences are then cleaved to release the mature enzymes in plastids. Thus, for manipulating a metabolic pathway involving monoterpene production, it may be beneficial to target GS, GR and GD (or some of them) to plastids. This targeting could be achieved by use of the native targeting sequences contained in the sequences of the native proteins, or by addition or exchange of heterologous sub-cellular targeting signals (leader sequences). Alternatively, the enzymes utilized in the methods of the invention could be directed to the cytoplasm by deletion of the plastid targeting signals. Methods for deletion, exchange and addition of nucleotide sequences are well known in the art, and can be readily used for manipulation of nucleotide segments encoding targeting signals of interest as described herein.

Thus, the nucleic acid construct of the present invention may also comprise an additional nucleic acid sequence encoding a leader peptide that allows transport of the polypeptide fused thereto to a sub-cellular organelle within the plant, cell wall or secreted to the extra-cellular matrix, as desired, such as plastids e.g., leucoplasts, chloroplasts and chromoplasts.

As used herein, the phrase “leader peptide” refers to a peptide linked in frame to the amino terminus of a polypeptide and directs the encoded polypeptide into a sub-cellular organelle of a cell (e.g. plastid, e.g. chloroplast). Such transit peptides are known in the art (see e.g. Clark et al. (1989) J. Biol. Chem. 264:17544-17550 and Della-Cioppa et al. (1987) Plant Physiol. 84:965-968).

According to specific embodiments the polypeptide further comprises a chloroplast leader peptide.

Chloroplast-leader sequences are known in the art and include, but not limited to, the targeting sequences of Arabidopsis ribulose bisphosphate carboxylase small chain; the small subunit of ribulose-1,5-bisphosphate carboxylase (Rubisco); 5-(enolpyruvyl) shikimate-3-phosphate synthase (EPSPS); tryptophan synthase; plastocyanin; chorismate synthase; and the light harvesting chlorophyll a/b binding protein (LHBP).

According to a specific embodiment the chloroplast leader peptide is of Arabidopsis ribulose bisphosphate carboxylase small chain (SEQ ID NO: 68).

According to a specific embodiment the chloroplast leader peptide is of Arabidopsis Chloroplast CTP2 sequence (SEQ ID NO: 91).

According to another embodiment, the chloroplast leader peptide is placed at the N-terminus of the polypeptide sequence.

According to specific embodiment, the peroxisome C-terminus tri-amino acid signal (SRL) is deleted from the amino acid sequence of the polypeptide, e.g. of Geraniol reductase (GR), in order to enable expression of the polypeptide in the chloroplast.

Selectable marker genes that allow for the detection of transformed cells and ensure maintenance of the vector in the cell can also be included in the expression vector. Preferred selectable markers include those which confer resistance to one or more drugs such as ampicillin, chloramphenicol, erythromycin, kanamycin (neomycin), hygromycin and tetracycline (Davies et al. (1978) Annu. Rev. Microbiol. 32:469). One non-limiting example of such a marker is the NPTII gene whose expression results in resistance to kanamycin or hygromycin, antibiotics which are usually toxic to plant cells at a moderate concentration (Rogers et al. in Weissbach A and Weissbach H, eds., Methods for Plant Molecular Biology, Academic Press Inc.: San Diego, Calif., 1988). Selectable markers can also allow a cell to grow on minimal medium, or in the presence of toxic metabolite and can include biosynthetic genes, such as those in the histidine, tryptophan, and leucine biosynthetic pathways.

Alternatively, or additionally, the presence of the desired construct in transformed cells can be determined by means of other techniques well known in the art, such as sequencing, PCR, Southern and Western blots.

Teachings of the invention further contemplate that the polynucleotides are part of a nucleic acid construct system where the GS, GR, GD and/or CR polypeptides are expressed from a plurality of constructs, as mentioned above.

According to one embodiment, the polynucleotides may be placed in any order in the nucleic acid construct or in the nucleic acid construct system.

Various construct schemes can be utilized to express few genes from a single nucleic acid construct. For example, the nucleic acid construct of some embodiments of the invention can include at least two promoter sequences each being for separately expressing a specific polypeptide. These at least two promoters which can be identical or distinct can be constitutive, tissue specific or regulatable (e.g. inducible) promoters functional in one or more cell types. Non-limiting examples for such constructs are described in Example 2A of the Examples section below, wherein GS was cloned downstream to CaMV 35S promoter (SEQ ID NO: 69); GR was cloned downstream to sgFiMV promoter (SEQ ID NO: 70); GD was cloned downstream to SVBV promoter (SEQ ID NO: 71) and CR is cloned upstream to the NPTII cassette, downstream to the NPTII cassette, upstream to the GS cassette or downstream to the GS cassette.

Alternatively, the genes can be co-transcribed as a polycistronic message from a single promoter sequence of the nucleic acid construct. To enable co-translation of all the genes from a single polycistronic message, the different polynucleotide segments can be transcriptionally fused via a linker sequence including an internal ribosome entry site (IRES) sequence which enables the translation of the polynucleotide segment downstream of the IRES sequence. In this case, a transcribed polycistronic RNA molecule including the coding sequences of two or three genes will be translated from both the capped 5′ end and the internal IRES sequence of the polycistronic RNA molecule to thereby produce the different polypeptides.

Still alternatively, each two polynucleotide segments can be translationally fused via a protease recognition site cleavable by a protease expressed by the cell to be transformed with the nucleic acid construct. In this case, a chimeric polypeptide translated will be cleaved by the cell expressed protease to thereby generate the different polypeptides.

According to specific embodiments the constructs are as described in Example 2A of the Examples section below.

The nucleic acid sequences and constructs of some embodiments of the invention can be introduced into cells by any one of a variety of known methods within the art. Such methods can be found generally described in Sambrook et al., [Molecular Cloning: A Laboratory Manual, Cold Springs Harbor Laboratory, New York (1989, 1992)]; Ausubel et al., [Current Protocols in Molecular Biology, John Wiley and Sons, Baltimore, Md. (1989)]; Chang et al., [Somatic Gene Therapy, CRC Press, Ann Arbor, Mich. (1995)]; Vega et al., [Gene Targeting, CRC Press, Ann Arbor Mich., (1995)]; Vectors [A Survey of Molecular Cloning Vectors and Their Uses, Butterworths, Boston Mass. (1988)] and Gilboa et al. [Biotechniques 4 (6): 504-512 (1986)] and include, for example, stable or transient transfection, lipofection, electroporation and infection with recombinant viral vectors.

Plant cells may be transformed stably or transiently with the nucleic acid constructs of some embodiments of the invention. The transformation process results in the expression of the heterologous nucleic acid sequence such as to change the recipient cell into a transformed, genetically modified or transgenic cell. In stable transformation, the nucleic acid molecule of some embodiments of the invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA includes two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112, U.S. Pat. Nos. 5,635,055; 5,824,877; 5,591,616; 5,981,840; and 6,384,301.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; Gordon-Kamm et al., Plant Cell 2:603-618, 1990; Fromm et al., Biotechnology 8:833-839, 1990; WO 93/07278; and Koziel et al., Biotechnology 11:194-200, 1993; U.S. Pat. Nos. 5,015,580; 5,550,318; 5,538,880; 6,160,208; 6,399,861; 6,403,865 4,945,050; 5,036,006; and 5,100,792; by polyethylene glycol (PEG)- or electroporation-mediated uptake (see, e.g., U.S. Pat. No. 5,384,253, EP 0292435, EP 0392225, and WO 93/07278); by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

Many vectors are available for transformation using Agrobacterium tumefaciens. These vectors typically carry at least one T-DNA border sequence and include vectors such as pCIB10, pBI121 and pBIN19 (Bevan, Nucl. Acids Res. 11:369, 1984). The binary vector pCIB10 contains a gene encoding kanamycin resistance for selection in plants and T-DNA right and left border sequences and incorporates sequences from the wide host-range plasmid pRK252 allowing it to replicate in both E. coli and Agrobacterium (Rothstein et al., Gene 53:153-161, 1987). Transformation of the target plant species by recombinant Agrobacterium usually involves co-cultivation of the Agrobacterium with explants from the plant and follows protocols well known in the art. The transformed tissue is regenerated on selectable medium carrying the antibiotic or herbicide resistance marker present between the binary plasmid T-DNA borders.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant.

Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by some embodiments of the invention.

Transient transformation can be effected by any of the direct DNA transfer methods described above or by viral infection using modified plant viruses.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria.

Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid.

The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of some embodiments of the invention can also be introduced into a plastid e.g. chloroplast genome thereby enabling chloroplast expression. Exemplary methods of transforming plastids are described in Svab et al., Proc. Natl. Acad. Sci. U.S.A. 90:913-917, 1993; Svab et al., Proc. Natl. Acad. Sci. U.S.A. 87:8526-8530, 1990; McBride et al., Proc. Natl. Acad. Sci. U.S.A. 91:7301-7305, 1994; Day et al., Plant Biotech. J. 9:540-553, 2011.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

According to a specific embodiment, following transformation the plant or plant cell is selected according to the level of expression of the heterologous polypeptide(s), and it is thus useful to ascertain expression levels in transformed plant cells, transgenic plants and tissue specific expression.

According to an alternative or an additional embodiment, following transformation the plant or plant cell is selected according to the monoterpene profile. Methods of evaluating monoterpene content are well known in the art and include gas chromatograph (GC), gas chromatography coupled to mass spectrometer (GC-MS), ion mobility spectrometers (IMS), high-field ion mobility spectrometers asymmetric waveform (FAIMS, high-field asymmetric waveform ion mobility spectrometry) electro-chemical sensors, electrochemical sensor arrays and colorimetric sensors.

Monoterpenes are volatile compounds, which are known to have a specific smell, for example, geraniol and citronellol have a rose-like scent, geranial, neral and citronellal have a strong lemon odor. Thus, according to another specific embodiment, following transformation the plant is selected according to the odor of the plant using methods known in the art such as employing a panel of human noses as sensors, artificial noses and electronic noses.

As used herein, the terms “heterologous polypeptide” or “recombinant polypeptide” refer to a polypeptide produced by recombinant DNA techniques, i.e., produced from cells transformed by an exogenous nucleic acid construct encoding the polypeptide. The recombinant polypeptide can be foreign to the cell or a homologous polypeptide derived from a nucleic acid sequence not from its natural location and expression level in the genome of the cell.

Thus, regardless of the method of introduction, the present teachings provide for an isolated cell (e.g., plant cell, e.g. woody plant cell) which comprises a heterologous nucleic acid sequence encoding any of the above mentioned enzymes such as GR, GS, GD, CR, GR+GD, GS+GR, GS+GD, GS+CR, GR+CR, GD+CR GS+GR+GD, GS+GR+CR, GS+GD+CR, GR+GD+CR or GS+GR+GD+CR. These heterologous sequences may be encoded by any of the above mentioned nucleic acid constructs.

The term “isolated” refers to at least partially separated from the natural environment e.g., from a whole plant.

The cell may be prokaryotic or eukaryotic such as bacterial, insect, fungal, plant or animal cell. According to a specific embodiment the cell is a plant cell.

Thus, according to an aspect of the present invention, there is provided a genetically modified plant e.g., woody plant or plant cell which comprises any of the above mentioned heterologously expressed enzymes.

As used herein, the term “genetically modified” refers to a cell (e.g. plant cell, e.g. woody plant cell) comprising a heterologous i.e., exogenous nucleic acid sequence. The cells may be transgenic or non-transgenic cells (i.e., wherein the cell doesn't comprise foreign regulatory elements such as viral components).

According to a specific embodiment, there is provided a woody plant or plant cell comprising a heterologous nucleic acid sequence encoding at least one polypeptide selected from the group consisting of Geraniol Synthase (GS), Geraniol Reductase (GR), Geraniol Dehydrogenase (GD) and Citral Reductase (CR).

The term “plant” as used herein encompasses whole plants, a grafted plant, ancestors and progeny of the plants and plant parts, including seeds, shoots, stems, roots (including tubers), rootstock, scion, and plant cells, tissues and organs. The plant may be in any form including suspension cultures, embryos, meristematic regions, callus tissue, leaves, gametophytes, sporophytes, pollen, and microspores. Plants that are particularly useful in the methods of the invention include all plants which belong to the superfamily Viridiplantee, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalypfus spp., Euclea schimperi, Eulalia vi/losa, Pagopyrum spp., Feijoa sellowlana, Fragaria spp., Flemingia spp, Freycinetia banksli, Geranium thunbergii, Ginkgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, trees. Alternatively algae and other non-Viridiplantae can be used for the methods of some embodiments of the invention.

According to specific embodiments the plant is a woody plant.

The term “woody plant” as used herein refers to a tree, namely a perennial plant having an elongated hard lignified stem. Woody plants include angiosperms and gymnosperm species and hybrids. Non-limiting examples of woody plants include eucalyptus, poplar, pine, fir, spruce, acacia, sweet gum, ash, birch, oak, teak, mahogany, sugar and Monterey, nut trees, e.g., walnut and almond, and fruit trees, e.g., apple, plum, citrus and apricot.

According to specific embodiments the woody plant is eucalyptus or poplar. Non-limiting examples of eucalyptus species include, but are not limited to, Eucalyptus alba, Eucalyptus bancroftu, Eucalyptus botyroides, Eucalyptus bndgesiana, Eucalyptus calophylla, Eucalyptus camaldulensis, Eucalyptus citriodora, Eucalyptus cladocalyx, Eucalyptus coccifera, Eucalyptus curtisii, Eucalyptus dalrympleana, Eucalyptus deglupta, Eucalyptus delagatensis, Eucalyptus diversicolor, Eucalyptus dunnu, Eucalyptus ficifolia, Eucalyptus globulus, Eucalyptus gomphocephala, Eucalyptus gunnu, Eucalyptus hemyi, Eucalyptus laevopinea, Eucalyptus macarthuru, Eucalyptus macrorhyncha, Eucalyptus maculata, Eucalyptus margmata, Eucalyptus megacarpa, Eucalyptus melhodora, Eucalyptus nicholu, Eucalyptus mtens, Eucalyptus nova-anglica, Eucalyptus obliqua, Eucalyptus obtusiflora, Eucalyptus oreades, Eucalyptus pauciflora, Eucalyptus polybractea, Eucalyptus regnans Eucalyptus resimfera, Eucalyptus robusta, Eucalyptus rudts, Eucalyptus sahgna, Eucalyptus sideroxylon, Eucalyptus stuartiana, Eucalyptus tereticornis, Eucalyptus torelhana, Eucalyptus urmgera, Eucalyptus urophylla, Eucalyptus viminalis, Eucalyptus viridis, Eucalyptus wandoo, Eucalyptus youmanni and hybrids thereof.

According to a specific embodiment the eucalyptus is not Eucalyptus citriodora.

Non-limiting examples of poplar (also known as populous, aspen and cottonwood) species include Populus tremula, Populus adenopoda, Populus alba, Populus canescens, Pacific Albus, Populus davidiana, Populus grandidentata, Populus sieboldii, Populus tremuloides, Populus deltoides, Populus fremontii, Populus nigra, Lombardy poplar, Regenerata poplar, Carolina poplar, Robusta poplar, Populus canadensis, Populus inopina, Populus angustifolia, Populus balsamifera, Populus generosa, Populus cathayana, Populus koreana, Populus laurifolia, Populus maximowiczii, Populus simonii, Populus suaveolens, Populus szechuanica, Populus trichocarpa, Populus tristis, Populus ussuriensis, Populus yunnanensis, Populus heterophylla, Populus lasiocarpa, Populus wilsonii, Populus euphratica, Populus ilicifolia, Populus guzmanantlensis, Populus mexicana and hybrids thereof.

According to specific embodiments the cell or the plant does not express detectable levels (e.g., by RT-PCR) of the GS, GR, GD and/or CR endogenously.

As used herein, the term “endogenous” refers to the expression of the native gene in its natural location and expression level in the genome of a cell.

It will be appreciated that when referring to a genetically modified plant or plant cell, the present inventors also refer to progeny arising therefrom.

Progeny resulting from breeding or from transforming plants can be selected, by verifying presence of exogenous mRNA and/or polypeptides by using nucleic acid or protein probes (e.g. antibodies). Alternatively, expression of the polypeptides of the present invention may be verified by measuring enhanced resistance to pest by infecting the genetically modified plant and a wild-type (i.e. non-modified plant of the same type) and comparing the disease in the plant as further described in details below.

Genetically modified plant cells may then be cultured in an appropriate medium to regenerate whole plants, using techniques well known in the art. These plants may then be grown, and either pollinated with the same transformed strain or different strains, and the resulting hybrid having the desired phenotypic characteristic identified. Two or more generations may be grown to ensure that the subject phenotypic characteristic is stably maintained and inherited and then seeds harvested to ensure the desired phenotype or other property has been achieved.

According to specific embodiments the development and growth of the genetically modified plant is not affected. Evaluating development and growth of a plant may be effected by determining e.g. fruit ripening, organ growth, cell division, cell elongation, senescence, germination, respiration, photosynthesis, transpiration, flowering, pollination and fertilization.

Thus, a single plant (whether transgenic or not) is transformed with nucleic acid construct or construct systems as described herein.

However, as the present teachings, relate to the expression of a plurality of transgenes, the transgenic plants or plant cells can be generated by crossing plants each expressing an individual transgene (or more) so as to obtain a hybrid product which comprises the plurality of transgenes.

Plants which are modified to express more than one transgene may be the outcome of crossing a first transgenic plant expressing transgene (a), with a second transgenic plant expressing transgene (b) and selection of a plant progeny which expresses transgenes (a+b). Thus, according to specific embodiments transgene (a) comprises GS and transgene (b) comprises GR, GD, CR, GR+GD, GR+CR or GD+CR. According to other specific embodiments transgene (a) comprises GR and transgene (b) comprises GD, GS, CR, GS +GD, GD +CR or GS +CR. According to other specific embodiments transgene (a) comprises GD and transgene (b) comprises GS, GR, CR, GS+GR, GS+CR or GR+CR. According to other specific embodiments transgene (a) comprises CR and transgene (b) comprises GS, GR, GD, GS+GR, GS+GD or GR+GD.

Thus, according to a specific embodiment, expressing the transgenes (e.g., GS+GR, GS+GD, GS+CR, GR+GD, GR+CR, GD+CR, GS+GR+GD, GS+GR+CR, GS+GD+CR, GR+GD+CR, GS+GR+GD+CR) is effected by the art of crossing and selection.

Crossing and breeding can be accomplished by any means known in the art for breeding plants such as, for example, cross pollination of the first and second plants that are described above and selection for plants from subsequent generations which express both the first and second enzymes. The plant breeding methods used herein are well known to one skilled in the art. For a discussion of plant breeding techniques, see Poehlman (1987) Breeding Field Crops. AVI Publication Co., Westport Conn. Many crop plants useful in this method are bred through techniques that take advantage of the plant's method of pollination.

As mentioned the present inventors have shown that the heterologous expression of GS, GD, GS and/or CR alters the plant monoterpene profile leading to the production of monoterpenes such as geraniol, geranial, neral, citronellol and citronellal that are found in minimal or undetectable quantities in most eucalyptus species.

Thus, according to an aspect of the present invention there is provided a method of enhancing at least one of geraniol, geranial, neral, citronellol and citronellal oil content of a woody plant, the method comprising expressing in the woody plant at least one recombinant polypeptide selected from the group consisting of Geraniol Synthase (GS), Geraniol Reductase (GR),Geraniol Dehydrogenase (GD) and Citral Reductase (CR), thereby enhancing at least one of geraniol, geranial, neral, citronellol and citronellal oil content of the woody plant.

As also noted, the present inventors have now uncovered that heterologous expression of GS, GD, GR and/or CR in eucalyptus enhances plant pest resistance. Without being bound by theory, it is suggested that as geraniol, geranial, neral, citronellol and citronellal are known to have pesticidal and repellant properties, this change in the monoterpene profile is the reason for the enhanced pest resistance.

Thus, according to specific embodiments the genetically modified woody plant disclosed herein is resistant to pest infection.

According to another aspect of the present invention there is provided a method of enhancing resistance of a woody plant to pest infection, the method comprising expressing in the woody plant at least one recombinant polypeptide selected from the group consisting of Geraniol Synthase (GS), Geraniol Reductase (GR), Geraniol Dehydrogenase (GD) and Citral Reductase (CR), thereby enhancing the resistance of the woody plant to pest infection.

According to a specific embodiment, the method further comprises growing the plant in a zone known to be at risk of infestation by the pest.

As used herein the term “pest” refers to an organism that negatively affect plants by colonizing, damaging, attacking, or infecting them. Thus, pests may affect the growth, development, reproduction, harvest or yield of a plant. This includes organisms that spread disease and/or damage the host and/or compete for host nutrients. Plant pests include but are not limited to fungi, bacteria, insects, and nematodes. According to specific embodiments the pest is an insect.

Non-limiting examples of pests include Roundheaded Borer such as long horned borers; psyllids such as red gum lerp psyllids (Glycaspis brimblecombei), blue gum psyllid, spotted gum lerp psyllids, lemon gum lep psyllids; tortoise beetles; snout beetles; leaf beetles; honey fungus; Thaumastocoris peregrinus; sessile gall wasps (Cynipidae) such as Leptocybe invasa, Ophelimus maskelli and Selitrichodes globules; Foliage-feeding caterpillars such as Omnivorous looper and Orange tortrix; Glassy-winged sharpshooter; and Whiteflies such as Giant whitefly. Other non-limiting examples of pests include Aphids such as Chaitophorus spp., Cloudywinged cottonwood and Periphyllus spp.; Armored scales such as Oystershell scale and San Jose scale; Carpenterworm; Clearwing moth borers such as American hornet moth and Western poplar clearwing; Flatheaded borers such as Bronze birch borer and Bronze poplar borer; Foliage-feeding caterpillars such as Fall webworm, Fruittree leafroller, Redhumped caterpillar, Satin moth caterpillar, Spiny elm caterpillar, Tent caterpillar, Tussock moths and Western tiger swallowtail; Foliage miners such as Poplar shield bearer; Gall and blister mites such as Cottonwood gall mite; Gall aphids such as Poplar petiolegall aphid; Glassy-winged sharpshooter; Leaf beetles and flea beetles; Mealybugs; Poplar and willow borer; Roundheaded borers; Sawflies; Soft scales such as Black scale, Brown soft scale, Cottony maple scale and European fruit lecanium; Treehoppers such as Buffalo treehopper; and True bugs such as Lace bugs and Lygus bugs.

As used herein the term “insect” refers to an insect at any stage of development, including an insect nymph and an adult insect. Non-limiting examples of insects include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Hemiptera.

According to specific embodiments the pest is selected from the group consisting of Glycaspis brimblecombei, Thaumastocoris peregrinus, Leptocybe invasa and Ophelimus maskelli.

The red gum lerp psyllid, Glycaspis brimblecombei (Gb) is a sap-sucking pest (Order Hemiptera: Psyllidae) that typically infect eucalyptus trees. Gb females lay between 45 and 700 eggs per lifetime. Gb Eggs hatch within 10 to 20 days and the emerging nymphs pierce eucalyptic tissue with their stylet (mouthparts), feeding on the xylem and phloem. As the nymphs feed on plant sugars from the leaves they secrete honeydew with which they construct a waxy protective cover (“lerp”) around themselves. The lerp is whitish and conical in shape and shelters insects during development, until they reach adult stage. Adults are about ⅛ inch long, slender, and light green to brownish with orangish and yellow blotches. Adults occur openly on foliage and do not live under lerp covers. Symptoms of Gb infestation include leaf loss and drying of lead shoots. Severe infestation can cause complete defoliation and death of trees.

Thaumastocoris peregrinus (hereinafter “Tp” or “Bronze bug”) is a sap-sucking pest (Order Hemiptera: Thaumastocoridae) that typically infect eucalyptus trees. The adult bronze bug is characterized by a strongly dorso-ventrally compressed and elongate body between 2-3.5 mm in length, a broad head, pedicellate eyes, and elongate conspicuous mandibular plates which are curved and broad on the outer margin. The body is light brown with darker areas. The eggs are dark, oval, with a sculptured chorion and a round operculum, on average 0.5 mm long and 0.2 mm wide. The crawlers and young nymphs are essentially orange, with black spots on the thorax and first abdominal segments. Symptoms of bronze bug infestation include leaf silvering, ranging from chlorosis to bronzing, heavy infestations cause leaves to become red/brown and defoliation occurs. Severe infestation may cause death of trees.

The Gall wasp Leptocybe invasa (Li) is a small wasp, brown in color with a slight to distinctive blue to green metallic shine that typically infect eucalyptus trees. The average female's length is 1.2 mm. Larvae are minute, white and legless. The adult female wasp lays her eggs on the midrib, petioles and stem of young trees, as well as on newly produced coppice growth and seedlings, resulting in the formation of bump-shaped galls. Five stages of gall development have been identified occurring prior to wasp emergence: 1) One to two weeks after egg laying, cork tissue appears at the egg insertion point and gall development begins within the plant tissue; 2) Development of a typical bump shape until the gall reaches its maximum size; 3) Fading of green colour on the gall surface, changing to a glossy pink colour; 4) Loss of gall glossiness with a colour change occurring to light or dark red; 5) Emergence holes of wasps visible. Symptoms of Li infestations include premature leaf fall, stunting, lodging and death of trees.

The gall wasp Ophelimus maskelli (Om) is a minute black insect whose larvae develop inside raised galls that form typically on eucalyptus leaves. Symptoms of Om infestations include appearance of slightly raised swellings, about 1 mm in diameter, on either side of the leaves, the galls are uniform in size and shape, hollow and each contains a tiny white grub. The Om gall wasp does not affect the long term health or vigour of the tree, but can affect its appearance.

As used herein “pest resistance” refers to resistance to the abundance and/or virulence of a pest at any step of the pest life cycle when it is associated with a host, including without limitation, colonization, reproduction, oviposition, galls formation, feeding, growth and mortality. Pest resistance is relative and is based on comparison with a control organism (e.g. plant) known to be resistant or sensitive.

As used herein, “resistant to pest” and “enhancing resistance” refer to an increase of at least 5% in the resistance of the genetically modified plant towards a pest in comparison to a suitable control e.g. a non-modified plant of the same species under the same developmental stage grown under the same conditions. According to a specific embodiment, the increase is in at least 10%, 30%, 40% or even higher say, 50%, 60%, 70%, 80%, 90% or more than 100%. Enhanced resistance to pests may be manifested in the form of reduced symptoms in a host, reduced number of viable pests on the plant surfaces, reduced number of eggs or egg clusters on the plants and/or retarded or altered growth development of nymphs.

According to specific embodiments the non-modified cell or plant does not express pesticidal effective amounts of GS, GR, GS and/or CR.

According to other specific embodiments the non-modified cell or plant does not comprise pesticidal effective amounts of geraniol, geranial, citronellol and/or citronellal.

Assays for testing pest resistance are well known in the art and mentioned hereinbelow.

Typically, for evaluating pest resistance, the plant of interest infested with a pest of interest is maintained in conditions sufficient to sustain health of the plant. For example, the plant is provided with adequate amount of water and maintained in sufficient temperature, lighting, and humidity conditions for proper growth, development, and/or maintenance of the plant.

Any of the following (or other) methods can be used to evaluate pest resistance: free-choice experiment and non-choice experiment.

In a free choice experiment setting the tested plant species (i.e. the wild type and the genetically modified plant) are exposed together to the pest, thus the pest can choose between the plant species.

In a non-choice experiment setting each of the tested plant species is separately and under the same conditions, inoculated with the pest.

Typically, evaluation of pest resistance is effected in pest proof cages which keep the inoculums in while preventing outside pests from entering the cage.

Pest resistance testing can be carried out on whole plants or on single leaves (see e.g. the clip-on insect cages described by University of Arizona Center for Insect Science Center for Education Outreach insected(dot)arizona(dot)edu/gg/resource/clip(dot)html). Exemplary experimental settings are described in Examples 5-7 of the Examples section which follows.

Following inoculation, symptoms are typically scored on a daily basis for several weeks (e.g. 1, 2, 3 or 4 months) by evaluating, for example, the number of live pests on each plant; the number of live pests not on plants; the number of dead pests; the number of deformed, dysfunctional or non-reproductive pests; the number of eggs and eggs clusters; the number of nymphs; the number of lerps; the number of galls; gall size; the number of vital larvae in galls; the number of defoliated leaves; the number of discolored leaves, the number of dead branches; and the number of dead plants.

According to some embodiments, there is provided a method of improving pest resistance of a grafted woody plant, the method comprising providing a scion that does not transgenically express the polypeptides of the present invention (e.g. GS, GR, GD and/or CR) and a plant rootstock that transgenically expresses at least one of GS, GR, GD and CR (in an abiotic stress responsive manner), thereby improving pest resistance of the grafted woody plant. In some embodiments, the plant scion is non-transgenic. Several embodiments relate to a grafted woody plant exhibiting improved pest resistance, comprising a scion that does not transgenically express the polypeptides of the present invention (e.g. GS, GR, GD and/or CR) and a plant rootstock that transgenically expresses at least one of GS, GR, GD and CR. In some embodiments, the plant root stock transgenically expresses at least one of GS, GR, GD and CR in a stress responsive manner.

According to another aspect of the present invention there is provided a pesticidal composition, comprising as an active ingredient the nucleic acid construct or construct system of the present invention; and an agriculturally acceptable carrier or diluent.

As used herein, the phrase “agriculturally acceptable carrier” refers to a carrier or a diluent that does not cause significant irritation to a plant and does not abrogate the biological activity and properties of the administered compound. Surfactants or other application-promoting adjuvants customarily employed in formulation technology are included under this phrase.

Suitable carriers and adjuvants can be solid or liquid and are the substances ordinarily employed in formulation technology, e.g. natural or regenerated mineral substances, solvents, dispersants, wetting agents, tackifiers, thickeners, binders, anti-freeze agents, preservatives or fertilizers. Such carriers are customarily employed in formulation technology and formulation techniques that are known in the art.

The composition may be in the form of any desired formulation such as a solution, emulsion, spray, suspension, powder, foam, oil dispersion, paste, granule, capsule or other finely or coarsely divided material or impregnant for natural or synthetic material.

As with the nature of the compositions, the methods of application, such as spraying, atomizing, dusting, scattering, coating or pouring, are chosen in accordance with the intended objectives and the prevailing circumstances.

The composition may be used alone or together with additional pesticides [e.g. Imidacloprid and DIPEL (Bacillus thuringiensis)].

Transgenic plants generated according to the above teachings are characterized by a modified oil composition. According to specific embodiments the genetically modified plant exhibits elevated levels of monoterpenes such as geraniol, geranial, neral, citronellol and/or citronellal while reduced relative levels of other monoterpenes.

According to an aspect of the present invention there is provided a method of producing oil, the method comprising providing the genetically modified woody plant of the present invention; and extracting the oil from the woody plant, thereby producing oil.

According to another aspect there is provided an oil produced according to the method.

According to yet another aspect of the present invention there is provided a eucalyptus oil having an increased content of at least one monoterpene selected from the group consisting of geraniol, geranial, neral, citronellol and citronellal; as compared to a eucalyptus oil of a non-genetically modified eucalyptus.

According to specific embodiments the eucalyptus oil having a reduced content of at least one monoterpene not selected from the group consisting of geraniol, geranial, neral, citronellol and citronellal; as compared to a eucalyptus oil of a non-genetically modified eucalyptus.

As used herein the term “oil” refers to an essential oil, a concentrated hydrophobic liquid containing monoterpenes extracted from a plant. As used herein the term “oil” includes derivatives thereof, including racemic mixtures, enantiomers, diastereomers, hydrates, salts, solvates, metabolites, analogs, and homologs. Composition, production and plant families of oils comprising monoterpenes (i.e. essential oils), are described in detail in Kirk-Othmer Encyclopedia of Chemical Technology, 4^{th} Edition S. Price, Aromatherapy Workbook—Understanding Essential Oils from Plant to Bottle, (HarperCollins Publishers, 1993; J. Rose, The Aromatherapy Book—Applications & Inhalations (North Atlantic Books, 1992); and in The Merck Index, 13^{th} Edition, each of which is incorporated herein by reference.

According to specific embodiments the oil is an eucalyptus oil.

As used herein the term “eucalyptus oil” refers to the essential oil from eucalyptus.

Essential oils are usually found in special secretory glands or cells within the plants and may be obtained from e.g. leaves, flowers, roots, buds, twigs, rhizomes, heartwood, bark, resin, seeds and fruits.

Methods of extracting oil from a plant are known in the art and include steam distillation, pressing fruit rinds, solvent extraction, macerating the flowers and leaves in fat and treating the fat with solvent, enfleurage and synthetically. See, e.g., Price, Aromatherapy Workbook—Understanding Essential Oils from Plant to Bottle (HarperCollins Publishers, 1993, the entire disclosure of which is incorporated herein by reference).

According to specific embodiments the monoterpene fraction is further purified from the oil.

According to specific embodiments, at least 20%, at least 30%, at least 40%, at least 50 at least 60, at least 70%, at least 80%, at least 90%, at least 95% or at least 99% of the oil is monoterpenes.

The monoterpene fraction may contain a single monoterpene or a mixture of monoterpenes.

According to specific embodiments the monoterpene fraction comprises at least one of geraniol, geranial, neral, citronellol and citronellal.

As used herein “at least one of geraniol, geranial, neral, citronellol and citronellal” refers to one, two, three, four or all five monoterpenes i.e. geraniol; geranial; neral; citronellol; citronellal; geraniol+geranial; geraniol+neral; geraniol+citronellol; geraniol+citronellal; geranial+neral; geranial+citronellol, geranial+citronellal; neral+citronellol; neral+citronellal; citronellol+citronellal; geraniol+geranial+neral; geraniol+geranial+citronellol; geraniol+geranial+citronellal; geraniol+neral+citronellol; geraniol+neral+citronellal; geraniol+citronellol+citronellal; geranial+neral+_citronellol; geranial+neral+citronellal; geranial+citronellol+citronellal; neral+citronellol+citronellal; geraniol+geranial+neral+citronellol; geraniol+geranial+neral+citronellal; geranial+neral+citronellol+citronellal; or geraniol+geranial+citronellol+citronellal; geraniol+neral+citronellol+citronellal; or geraniol+geranial+neral+citronellol+citronellal.

According to specific embodiments, a specific monoterpene comprises at least 5%, at least 10%, at least 20% at least 30%, at least 40%, at least 50%, at least 60%, at least 70%, at least 80%, at least 90%, at least 95% or more say 100% of the monoterpene fraction.

Methods of purifying a monoterpene fraction from oil are known in the art and include for example gas chromatography (GS), GS- mass spectrometer (GC-MS), repeated distillation or other method such as disclosed in U.S. Pat. Nos. 8,507,734, 5,094,720 and 7,727,401.

According to specific embodiments, at least 70%, at least 80%, at least 90%, at least 95% or at least 99% of the purified fraction is monoterpenes.

As monoterpenes are volatile compounds the monoterpene fraction may be collected directly from the plant by method known in the art such as disclosed in Yang et al. Metabolic Engineering 13 (2011) 414-425; Ohara et al. Plant Biotechnology Journal (2010) 8, pp. 28-37; Aharoni et al. The Plant Cell, (2003) 15: 2866-2884; Lucker et al. Plant Physiology, (2004) 134: 510-519; Diemer et al. Plant Physiol. Biochem. (2001) 39: 603-614; and Gutensohn et al. The Plant Journal (2013) 75,351-363.

Thus, according to another aspect of the present invention there is provided a method of producing at least one monoterpene selected from the group consisting of geraniol, geranial, neral, citronellol and citronellal, the method comprising providing the genetically modified woody plant of the present invention, and extracting the monoterpene from the woody plant, thereby producing at least one monoterpene selected form the group consisting of geraniol, geranial, neral, citronellol and citronellal.

Also contemplated are processed products of the plants (e.g., woody plants) of some embodiments of the invention including but not limited to ornament, timber or firewood, charcoal, pellet, pulp, paper, sawmill, furniture, construction materials, dyes, mulch, fertilizers, as well as nectar for honey and oil for pest repellant, mosquito repellent, pesticides, fuel, food, feed, beverage, sweets, toothpaste, cosmetics, perfume, soap, detergents, antiseptic, medicinal and pharmaceutics industries.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Geraniol and Citronellal Increase Eucalyptus Camaldulensis Resistance to Ophelimus Maskelli Infection

Materials and Methods

Preparation of monoterpene disks—100 μl of pure monoterpene solutions of geraniol, citronellal, eucalyptol (also known as 1,8-cineole, 1,8-epoxy-p-menthane) or mineral oil (Sigma-Aldrich Cat. Nos. G9698, 27470, C80601 or M5904, respectively) were each mixed with 5 ml of Vaseline. The mixed solutions were then spread using a spatula onto 20 mm in diameter filter paper disks (Whatman 3 mm cellulose chromatography papers, Sigma-Aldrich Cat. No. Z742422).

Free choice experiments—The free-choice experiments, were gall wasps were free to choose which plant to infest in a given cage, were carried out with Ophelimus maskelli gall wasp in three net cages of 80 cm×100 cm×150 cm in a greenhouse (28 ±2° C.) as described in Bleeker et al. [Plant Physiol. (2009) 151(2): 925-935], with the following modifications. Four Eucalyptus camaldulensis 3 months old saplings were placed randomly in each cage and 10 disks with either geraniol, citronellal, eucalyptol (1,8-Cineole, used as control) or mineral oil (used as control) were attached by metal wires to the branches of each sapling (10 branches for each sapling) (see FIG. 2) so that each sapling had 10 disks attached with one of the treatments. Next, 500 Ophelimus maskelli female adult wasps were released in the middle of each net cage. One week later the disks were removed from the branches of the saplings, the saplings were taken out of the cage and thereafter the Eucalyptus saplings were transferred to an insect free net house, an outdoor perimeter surrounded by a 50 mesh insect proof net. The plants were maintained in the net house for 6 weeks after inoculation. At the end of the six week period, infection levels were evaluated by counting the number of galls developed on the treated saplings.

Results

To test the effect of geraniol and citronellal on Eucalyptus camaldulensis pest resistance, saplings were treated with filter papers applied with geraniol, citronellal, mineral oil control or eucalyptol (an abundant monoterpene oil in most eucalyptus species) control and subjected to Ophelimus maskelli infection in a free choice experiment setting. As can be seen in Table 1 below and FIG. 3, treatment with eucalyptol did not have a negative effect on Ophelimus maskelli oviposition and even resulted in a slight increase in the number of infected leaves as compared to treatment with mineral oil. In sharp contrast, treatment with geraniol or citronellal resulted in significantly decreased oviposition of Ophelimus maskelli as manifested by both reduced number of infected leaves and reduced number of galls per leaf in comparison to both the mineral oil control and the eucalyptol control.

Taken together, treatment with geraniol or citronellal significantly increased Eucalyptus camaldulensis resistance to Ophelimus maskelli infection.

TABLE 1
Results of the free-choice experiment

| Cage | | Number of infected leaves | | | |
|---|---|---|---|---|---|
| No. | treatment | 1-10 | 10-50 | 50-100 | total |
| 1 | mineral | 2 | 9 | 0 | 11 |
| | oil | | | | |
| | Eucalyptol | 4 | 14 | 6 | 24 |
| | citronellal | 1 | 4 | 0 | 5 |
| | geraniol | 4 | 3 | 1 | 8 |
| 2 | mineral | 4 | 8 | 3 | 15 |
| | oil | | | | |
| | Eucalyptol | 5 | 6 | 6 | 17 |
| | citronellal | 1 | 2 | 0 | 3 |
| | geraniol | 4 | 2 | 1 | 7 |
| 3 | mineral | 4 | 14 | 2 | 20 |
| | oil | | | | |
| | Eucalyptol | 7 | 10 | 6 | 23 |
| | citronellal | 1 | 3 | 0 | 4 |
| | geraniol | 4 | 1 | 1 | 6 |
| average | mineral | 3.3333333 | 10.333333 | 1.666667 | 15.3333333 |
| | oil | | | | |
| | eucalyptol | 5.3333333 | 10 | 6 | 21.3333333 |
| | citronellal | 1 | 3 | 0 | 4 |
| | geraniol | 4 | 2 | 1 | 7 |

### Example 2A

### Cloning and Transformation of Eucalyptus Plants with Geraniol Synthase, Geraniol Reductase, Geraniol Dehydrogenase and/or Citral Reductase

Materials and Methods

Cloning—Geraniol Synthase (GS) from Ocinum basilicum (SEQ ID NO: 36), Geraniol reductase (GR) from S. cerevisiae (SEQ ID NO: 47) and Citronellol dehydrogenase (GD) from Ocinum basilicum (SEQ ID NO: 58) coding sequences and expression cassettes were synthetically synthesized and cloned into pBI121 binary vector (GenBank: AF485783.1) between the right border and the left border of the T-DNA. The coding sequences of each enzyme were optimized according to Eucalyptus Grandis codon usage. Each binary vector contained the NPTII selection gene (SEQ ID NO: 72). Citral reductase (CR), i.e. Alkene reductase GTE1 from Gluconobacter oxydans bacteria (WP_011252080) (SEQ ID NO: 79) coding sequence was cloned into NPTII cassette.

Five different constructs were synthesized (FIG. 4): construct A for GS expression; construct B for GS and GR expression; construct C for GS, GR and GD expression and construct D for GS and GD expression and construct E for expression of GS+GD+CR.

In all constructs, GS was cloned downstream to Cauliflower mosaic virus (CaMV) 35S promoter (SEQ ID NO: 69) and Omega 5′-UTR.

In construct A, GS was cloned upstream to nopaline synthase (NOS) gene terminator (SEQ ID NO: 64) and in constructs B, C and D, GS was cloned upstream to agropine synthase gene terminator (AGS) (SEQ ID NO: 66).

In constructs B and C, GR was cloned downstream to Figwort mosaic virus subgenomic transcript promoter (sgFiMV) (SEQ ID NO: 70) and Omega 5′UTR.

In construct B, GR was cloned upstream to NOS terminator (SEQ ID NO: 64) and in constructs C upstream to octapine synthase gene terminator (OCS) (SEQ ID NO: 67).

In constructs C and D, GD was cloned downstream to Strawberry vein banding virus (SVBV) promoter (SEQ ID NO: 71) and the Tobacco etch viral 5′-UTR, upstream to NOS terminator (SEQ ID NO: 64).

In construct E, Citral reductase, i.e. Alkene reductase GTE1 from Gluconobacter oxydans bacteria (WP_011252080) (SEQ ID NO: 79) coding sequence was cloned upstream the NPTII cassette, downstream the NPTII cassette, upstream the GS cassette and downstream the GS cassette.

Transformation and selection—Agrobacterium EAH105 was electro-transformed with constructs A, B, C, D or E, selected for 48 hours on kanamycin plates (100 μg/ml), and used for Eucalyptus Urophyla (E. Urophyla)×Eucalyptus Tereticornis (E. Tereticornis) hybrids transformation. Transformation was effected using a protocol essentially as described in Prakash et al., In Vitro Cell Dev Biol.—Plant 45:429-434, 2009. Briefly, shoots of E. Urophyla×E. Tereticornis hybrids were propagated in vitro on Murashige and Skoog (MS) basal salt medium consisting of 3% (w/v) sucrose and 0.8% (w/v) agar. Transgenic plant selection was performed using kanamycin in whole single shoots in the selection plates by standard protocols. Wild type (WT) E. Urophyla×E. Tereticornis hybrids or E. Urophyla×E. Tereticornis hybrids transformed with empty pBI121 vector served as control. Plants leaves were then analyzed by PCR to confirm the presence of the T-DNA in the genome and by RT-PCR to detect GS, GR, GD and/or CR transcripts using specific primers, SEQ ID NOs: 73-74, 75-76 and 77-78, respectively. Positive plants were later rooted and propagated by standard protocols. Five different transformation events were selected from each construct for further analysis.

### Example 2B

### Cloning and Transformation of Eucalyptus Plants with Geraniol Reductase from Plant Origin

Materials and Methods

Cloning—Geraniol reductase (GR) was obtained from plants. Specifically, 12-Oxophytodienoate Reductase from Rubber tree (Hevea brasiliensis) (SEQ ID NO: 82), from Arabidopsis thaliana (SEQ ID NO: 84), from Solanum lycopersicum (Tomato) (SEQ ID NO: 86), from Eucalyptus grandis (SEQ ID NO: 88), or from Rosa multiflora (SEQ ID NO: 90) coding sequences and expression cassettes were synthetically synthesized and cloned into pBI121 binary vector (GenBank: AF485783.1) between the right border and the left border of the T-DNA.

The peroxisome C-terminus tri-amino acid signal (SRL) is deleted from the each of the coding sequences. A Chloroplast transit peptide is added to the N-terminus of the protein sequences (SEQ ID NO: 91).

Moreover, Geraniol Synthase (GS) from Ocinum basilicum (SEQ ID NO: 36) and Citronellol dehydrogenase (GD) from Ocinum basilicum (SEQ ID NO: 58) coding sequences and expression cassettes were synthetically synthesized and cloned into pBI121 binary vector (GenBank: AF485783.1) between the right border and the left border of the T-DNA, as described in detail in Example 2A above. Citral reductase, i.e. Alkene reductase GTE1 from Gluconobacter oxydans bacteria (WP_011252080) (SEQ ID NO: 79) coding sequence was cloned into NPTII cassette, as described in detail in Example 2A above. The coding sequences of each enzyme were optimized according to Eucalyptus Grandis codon usage. Each binary vector contained the NPTII selection gene (SEQ ID NO: 72).

Five different constructs were synthesized, as described in detail in Example 2A above and in FIG. 4.

Transformation and selection—Agrobacterium EAH105 was electro-transformed with constructs A, B, C, D or E, as described in detail in Example 2A above.

Results

Conversion of Geraniol to Citronellol is tested using Geraniol reductase (GR) enzymes derived from plant origins. Different reductases are being tested. The first, 12-Oxophytodienoate Reductase from Rubber tree (Hevea brasiliensis) (SEQ ID NO: 81) is used which was previously shown to convert Geraniol to Citronellal in vitro [Yuan T. T. et al., Natural products and bioprospecting (2011) 1(3): 108-111]. Additionally, 12-Oxophytodienoate Reductase homologs from other plant origins are tested (SEQ ID NOs: 83, 85, 87 and 89). In order to direct expression of the reductase in the chloroplast, a Chloroplast transit peptide is added to the N-terminus of the protein (SEQ ID NO: 91) and the peroxisome C-terminus tri-amino acid signal (SRL) is deleted. Plant derived GR (e.g. rubber tree GR) are expressed in Eucalyptus plants and analyzed for monoterpenes profile.

Of note, DNA sequences are optimized to Eucalyptus codon usage as published by the codon usage database—www(dot)kazusa(dot)or(dot)jp/codon/. Eucalyptus codon usage is also generated by counting each codon rate from a full eucalyptus transcriptome library. The present inventors also make use of computer software that has the feature of reverse translation to get the optimized DNA.

### Example 2C

### Expression of Citral Reductase in Eucalyptus Trees

Materials and Methods

Cloning—Geraniol reductase (GR) was obtained from plants or from single cell organisms and cloned as described in detail Examples 2A and 2B, above.

Moreover, Geraniol Synthase (GS) and Citronellol dehydrogenase (GD) were cloned as described in detail Example 2A, above. Citral reductase, i.e. Alkene reductase GTE1 from Gluconobacter oxydans bacteria (WP_011252080) (SEQ ID NO: 79) coding sequence was cloned into NPTII cassette, as described in detail in Example 2A above. The coding sequences of each enzyme were optimized according to Eucalyptus Grandis codon usage. Each binary vector contained the NPTII selection gene (SEQ ID NO: 72).

Five different constructs were synthesized, as described in detail in Example 2A above and in FIG. 4.

Transformation and selection—Agrobacterium EAH105 was electro-transformed with constructs A, B, C, D or E, as described in detail in Example 2A above.

Results

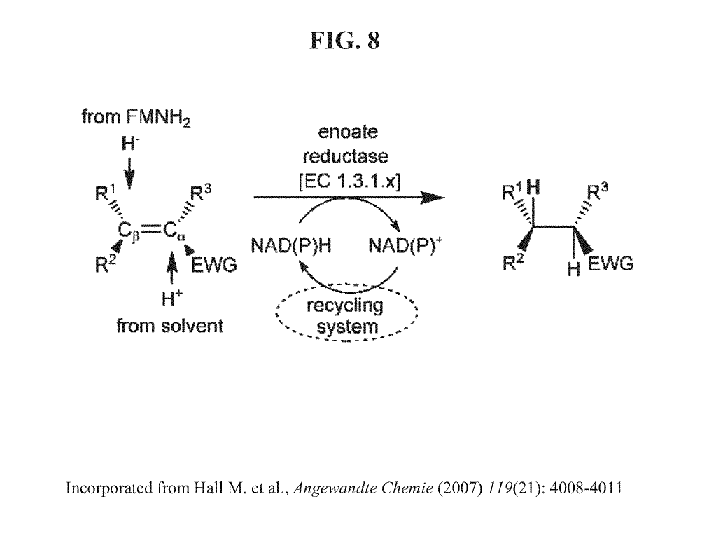

The literature teaches that Citral reductase can convert citral substrates in vitro into citronellal. The present inventors suggest that Reductase enzymes may be able to convert in vivo Geraniol to Citronellol and Citral to Citronellal by reducing the double C=C bond resulting in C—C(—OH)=O, as depicted in FIG. 8.

Conversion of Citral to Citronellal by Citral reductase is tested using Alkene reductase GTE1 from Gluconobacter oxydans bacteria (WP_011252080) (SEQ ID NO: 79) that was previously shown to convert citral to citronellal in vitro [Yin B. et al., Molecular biotechnology (2008) 38(3): 241-245]. In order to direct expression of the reductase in the chloroplast, a Chloroplast transit peptide is added to the N terminus of the protein (SEQ ID NO: 91). GTE1 from Gluconobacter oxydans bacteria is expressed constitutively with GS and analyzed for monoterpenes profile.

Of note, DNA sequences are optimized to Eucalyptus codon usage as published by the codon usage database—www(dot)kazusa(dot)or(dot)jp/codon/. Eucalyptus codon usage is also generated by counting each codon rate from a full eucalyptus transcriptome library. The present inventors also make use of computer software that has the feature of reverse translation to get the optimized DNA.

### Example 3

### Heterologous Expression of GS, GR and/or GD Modifies Eucalyptus Plants Monoterpene Profile

Materials and Methods

Extraction of oil—Essential oil was obtained from 2 months old wild-type (WT) and transgenic plants. Specifically, fresh leaves were weighed and 0.2-0.4 g were placed in a glass vial and kept at −20° C. For water content calculation, an additional leaf from each plant was weighed, dehydrated in 60° C. for 48 hours and weighed again. Monoterpene extraction was performed with dichloromethane and followed by sonication. As an internal standard, biphenyl was added to all samples.

Gas Chromatography—Mass spectrometry (GC-MS) analysis—GC analyses were performed with a Shimadzu GC-14B gas chromatograph containing a flame ionization detector using a Supelco wax column (60 m×0.25 mm i.d., film thickness 0.25 μm) according to the following program: 70° C. for 4 minutes, ramp at 4° C./min to 220° C. for 5 minutes; carrier gas, N₂. The relative amounts of the different constituents were determined by computer-based calculation of peak area normalization without any correction factor. Peaks obtained were compared with the data obtained from GC-MS.

MS analyses were performed with a Q Mass 910 Perkin-Elmer mass spectrophotometer equipped with fused silica (BP 21) capillary columns (30 m×0.25 mm i.d., film thickness 0.25 μm). Analytical conditions were as follows: injector and detector temperatures were 230° C. and 250° C., respectively; oven temperature was programmed from 40° C. (isothermal for 7 minutes) to 190° C. (isothermal for 20 minutes) at 5° C./min; carrier gas, helium. The compounds were identified on the basis of computer matching of mass spectra using the library search system HP-5872 (Hewlett-Packard) [Batish et al. 2006].

The chemical composition of Eucalyptus oil was determined by GC—MS and a flame ionization detection (FID) detector fitted with a 60 m×0.25 mm×0.25 m WCOT column coated with diethylene glycol (AB-Innowax 7031428, Japan). Carrier gas was helium with a flow rate of 3 ml/min at a column pressure of 155 kPa. Both injector and detector temperatures were maintained at 260° C. Samples (0.2 ml) were injected into the column with a split ratio of 80:1. Component separation was achieved following a linear temperature program of 60-260° C. at 3° C./min and then held at 260° C. for 10 minutes, with a total run time of 40 minutes. The percentage composition was calculated using area normalization method assuming equal detector response. Quantification was performed by comparing peak area calculations to standard curves done with standards (geraniol, citronellal, eucalyptol or citral Sigma-Aldrich cat no: G9698, 27470, C80601, C83007, respectively). The samples were then analyzed on same Shimadzu instrument fitted with the same column and following the same temperature program as above. The MS parameters used were: ionization voltage (EI) 70 eV, peak width 2 s, mass range 40-850 m/z and detector voltage 1.5 V. Analytes profile was characterized from their mass spectral data using National Institute of Standards and Technology (NIST12 or NIST62) and Wiley 229 mass spectrometry libraries [Kumar et al., 2012].

Results

To test the effect of heterologous expression of GS, GR and/or GD on the monoterpene profile of Eucalyptus plants, essential oils extracted from wild-type (WT) and transgenic plants transcribing constructs A, B, C or D are analysed by GC-MS.

As can be seen in FIG. 5, essential oil extracted from leaves of wild type Eucalyptus Urophyla×Tereticornis hybrid plants (WT) contained high levels of eucalyptol and α-pinene with insignificant amounts of geraniol, geranial (alpha citral) and neral (beta citral). Distinctively, transgenic plants transformed with construct C (encoding GS, GR and GD, event POC-1-9A) had a different fragrance with stronger lemon-like smell as compared to the WT as indicated by a panel of lab personnel by smell and personal impressions. Indeed, essential oil extracted from leaves of the transgenic plants (event POC-1-9A) contained elevated levels of geraniol, geranial and neral (1.27, 0.56 and 0.34 mg/gr dry weight, respectively) and decreased levels of eucalyptol and α-pinene. Interestingly, essential oil extracted from leaves of both the WT and transgenic plants contained no trace of citronellal.

Plants expressing the GS enzyme (construct A) contain higher concentrations of geraniol as compared to the WT. Plants expressing GS and GR enzymes (construct B) contain elevated levels of geraniol and citronellol as compared to the WT. Plants expressing GS and GD enzymes (construct D) contain higher geraniol and citral concentrations.

### Example 4

### Heterologous Expression of GS Enhances Expression of Monoterpenes in Eucalyptus Plants

Materials and Methods

Cloning—Geraniol Synthase (GS) from Ocinum basilicum (SEQ ID NO: 36) coding sequence and expression cassette was synthetically synthesized and cloned as described in Example 2A, hereinabove.

Transformation and selection—was carried out as described in Example 2A, hereinabove.

Extraction of oil—was carried out as described in Example 3, hereinabove.

Gas Chromatography—Mass spectrometry (GC-MS) analysis—GC analyses were performed as described in Example 3, hereinabove.

Specifically, leaf tissues from each of the three lines generated were used for GC-MS analysis. Terpenes identification was done by MS and quantitative analysis was performed using FID responses compared to standard curve generated by known concentrations of standards. Biphenyl was used as internal standard. Wild type non transgenic eucalyptus was used as control.

Real Time PCR (qPCR)

RNA extraction was done with Plant/Fungi Total RNA Purification Kit (NorgenBiotek Cat#25800) as describe in the manufacture protocol. One plant of each line was tested. cDNA was extracted using High capacity cDNA reverse transcription kit (Applied Biosystems). cDNA was diluted with double distilled water (1:1) and 2 μl were used for each reaction. Each sample was reacted with primers for GS gene (provided in Example 2, hereinabove). For each sample 3 technical repeats were performed. Gene expression of the tested gene was compared to expression level of housekeeping gene TEF (elongation factor) to lower the effect of technical errors and it is presented as 2^{-ΔCT}(ΔCT=CT(reference gene)−CT(target gene)).

Results

Eucalyptus trees expressing Geraniol synthase (GS) were analyzed for monoterpenes profile (by GCMS) and gene expression level (by qPCR). Three transgenic events (A, B and C) were tested for each.

As illustrated in FIG. 6, Geraniol, cis-citral and trans-citral monoterpenes were produced in all the GS transgenic lines but not in WT, while the concentration of monoterpenes that are naturally produced in eucalyptus (alpha-Pinene, Limonene and Eucalyptol) were higher in WT than in transgenic plants.

Real Time PCR results indicated that GS gene was expressed in all events (FIG. 7). GS expression was detected in all the tested events and was about 10 times higher than an endogenous random monoterpene synthase (FIG. 7).

Taken together, efficient expression of GS in Eucalyptus trees resulted in a marked production of Geraniol, cis-citral and trans-citral monoterpenes.

### Example 5

### Heterologous Expression of GS, GR, GD and/or CR Protects Eucalyptus Plants from Glycaspis Brimblecombei Infection

Materials and Methods

Whole plant assay×3 months old WT, empty vector control (control) and 5 independent transformation events of transgenic Eucalyptus Urophyla×Tereticornis hybrids plants of each line are grown in a green house at 24° C., 40-60% relative humidity (RH) and 16 hours of light per day. Each plant line is maintained in a separate insect proof cage and each plant is inoculated with 50 adult and/or nymphs bugs that are pre-reared in culture. The following parameters are recorded every day for 40 days after Gb inoculation:

1. Number of live bugs on each plant;

2. Number of live bugs not on plants;

3. Number of dead bugs;

4. Number of deformed, dysfunctional or non-reproductive pests;

5. Number of eggs laid;

6. Number of nymph hatched;

7. Number of lerps;

8. Number of defoliated leaves;

9. Number of dead branches;

10. Number of dead plants.

Single leaf assay—Five of each 3 months old WT, control and transgenic Eucalyptus Urophyla×Tereticornis hybrids plants of each line are grown in a green house at 24° C., 40-60% RH and 16 hours of light per day. Each line is contained in a separate insect proof cage and 5 leaves of each plant are covered with clip-on insect cages described by University of Arizona Center for Insect Science Center for Education Outreach insected(dot)arizona(dot)edu/gg/resource/clip(dot)html. Ten adult insects are placed inside each leaf clip cage. Clip cages can be clipped over a leaf-feeding insect without disturbing the insect or the plant. These cages provide a simple way to isolate one or more sap-sucking pests or other small insects for investigation and observation.

The following parameters are recorded every day for 40 days after Gb inoculation:

1. Percent mortality ((total number of insects−live insects)/total number of insects)×100;

2. Extent, number and percentage of discolored leaves;

3. Number of eggs or egg clusters.

Results

To evaluate the ability of heterologous expression of GS, GR, GD and/or CR to protect Eucalyptus plants from Glycaspis brimblecombei (Gb) infection WT and transgenic Eucalyptus Urophyla×Tereticornis hybrids plants expressing constructs A, B, C, D or E are grown in insect proof cages in the greenhouse together with nymph and/or adult Gb. The insect proof cages keep the inoculums in while preventing outside pests from entering the cage. Following Gb inoculation, the appearance of lerps, which compete with the plant for photosynthesis products and plant synthesized sugars, is evaluated on the upper or lower surface of the leaves. Plants are further examined to determine the number of Gb eggs and clusters of eggs on the plant tissues including leaves, reproductive organs, branches and stems. The primary endpoints for a resistant plant can be reduced symptoms, reduced number of viable pests on the plant surfaces, reduced number of eggs or egg clusters on the plants and/or retarded or altered growth development of nymphs. In some cases resistant plants may simply cause the contacting pests to become unviable or sterile without causing pest death.

Transgenic plants transcribing the constructs exhibit fewer symptoms, fewer vital Gb specimens, less eggs and less egg clusters, and/or less newly hatched nymphs, compared to controls. In addition transgenic plant lines are more resistant to Gb infection showing less plant growth inhibition, less leaf and other tissue damage such as lerps compared to control and wt plants that are infected with Gb.

### Example 6

### Heterologous Expression of GS, GR, GD and/or CR Protects Eucalyptus Plants from Thaumasocoris Pregrinus Infection

Materials and Methods

Whole plant assay—As described in Example 5 hereinabove. The following parameters are recorded every day for 40 days after Thaumastocoris peregrinus inoculation:

1. Number of live bugs on each plant;

2. Number of live bugs not on plants;

3. Number of dead bugs;

4. Number of deformed, dysfunctional or non-reproductive pests;

5. Number of laid eggs;

6. Number of nymph hatched;

7. Number of defoliated leaves;

8. Number of discolored leaves;

9. Number of discolored patches per infected leaf;

10. Number of dead branches;

11. Number of dead plants.

Single leaf assay—As described in Example 5 hereinabove. The following parameters are recorded every day for 40 days after Thaumastocoris peregrinus inoculation:

1. Percent mortality ((total number of bugs−live bugs)/total number of bugs)×100;

2. Extent, number and percentage of discolored leaves;

3. Number of eggs or egg clusters.

Results

To evaluate the ability of heterologous expression of GS, GR, GD and/or CR to protect Eucalyptus plants from Thaumastocoris peregrinus (Tp or Bronze bug) infection WT and transgenic Eucalyptus Urophyla×Tereticornis hybrids plants expressing constructs A, B, C, D or E are grown in insect proof cages in the greenhouse together with nymph and/or adult Bronze bugs. The insect proof cages keep the inoculums in while preventing outside pests from entering the cage. Following Bronze bug inoculation, the appearance of leaf damage is evaluated. Leaf damage can be seen as bronze-like spots or areas on the upper or lower surface of the leaves. These bronze areas are formed as a direct and/or an indirect result of the sap-sucking activities of the Bronze Bugs. Plants are further examined to determine the number of Bronze bugs eggs and clusters of eggs on the plant tissues including leaves, reproductive organs, branches and stems and the number of dead or dysfunctional Bronze bug specimens found on or adjacent to the plants. The primary endpoints for a resistant plant can be reduced symptoms, reduced number of viable pests on the plant surfaces, reduced number of eggs or egg clusters on the plants and/or retarded or altered growth development of nymphs. In some cases resistant plants may simply cause the contacting pests to become unviable or sterile without causing pest death.

Transgenic plants transcribing the constructs exhibit fewer symptoms, fewer vital Bronze bugs, less eggs and less egg clusters and/or less newly hatched nymphs, compared to controls. In addition, transgenic plant lines are more resistant to Bronze bug infection showing less leaf and other tissue damage, compared to control and wt plants that are infected with Bronze bugs.

### Example 7

### Heterologous Expression of GS, GR, GD and/or CR Protects Eucalyptus Plants from Leptocybe Invasa and Ophelimus Maskelli Infection

Materials and Methods

Non-Choice bioassay—WT, empty vector control and 5 independent transformation events of transgenic Eucalyptus Urophyla×Tereticornis hybrids plants of each line are grown in insect proof cages in a green house at 24° C., 40-60% RH and 16 hours of light per day. 20 adult gall wasps are placed inside a leaf cage made out of a falcon 50 ml tube with a mesh lid. Each leaf cage is attached to one leaf of the transgenic or WT tree using a clip. 3 leaf cages are attached to each plant. The leaf cages are removed 6 days following inoculation, after all the adults die. The number of galls, gall size, vital larvae per 10 galls and emerging adults (by the exit hole) are recorded 1, 2, 3 and 4 months after inoculation.

Results

To evaluate the ability of heterologous expression of GS, GR, GD and/or CR to protect Eucalyptus plants from Leptocybe invasa (Li) or Ophelimus maskelli (Om) infection, WT and transgenic E. Urophyla×E. Tereticornis hybrids plants expressing constructs A, B, C, D or E are grown in insect proof cages in the greenhouse together with adult gall wasps. The insect proof cages keep the inoculums in while preventing outside pests from entering the cage. Following wasp inoculation, the appearance of galls in the veins and in the leaves is evaluated. Plants are examined to determine number of galls, gall size (maximum length), number of vital larvae in galls and the number of emerging matured gall wasps.

Transgenic plants transcribing the constructs exhibit fewer galls of smaller sizes, compared to controls. In addition, in transgenic plant lines less or no vital larvae are detected in the small galls and less or no adult wasps will emerge compared to the controls. Thus, the transgenic lines are more resistant to both Li and Om gall wasp infection as compared to control and wt plants that are infected with fully developed galls.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### References

(other references are cited in the application)
1. Bayrak A (1994) Volatile oil composition of Turkish rose (Rosa damascena). J Sci Food Agric 64: 441-448.
2. Antonelli A, Fabbri C, Giorgioni M E, Bazzocchi R (1997) Characterization of 24 old garden roses from their volatile compositions. J Agric Food Chem 45: 4435-4439.
3. Rao B R R, Sastry K P, Saleem S M, Rao E V S P, Syamasundar K V, Ramesh S (2000) Volatile flower oils of three genotypes of rose-scented geranium (Pelargonium sp.). Flavour Fragr J 15: 105-107.
4. Charles D J, Simon J E (1992) A new geraniol chemotype of Ocimum gratissimum L. J Essential Oil Res 4: 231-234.
5. Mallavarapu G R, Rao B R R, Kaul P N, Ramesh S, Bhattacharya A K (1998) Volatile constituents of the essential oils of the seeds and the herb of palmarosa (Cymbopogon martinii (Roxb.) Wats. var. motia Burk.). Flavour Fragr J 13: 167-169.
6. Mockute D, Bernotiene G (1999) The main citral-geraniol and carvacrol chemotypes of the essential oil of Thymus pulegioides L. growing wild in Vilnius district (Lithuania). J Agric Food Chem 47: 3787-3790.
7. Vieira R F, Grayer R J, Paton A, Simon J E (2001) Genetic diversity of Ocimum gratissimum L. based on volatile oil constituents, flavonoids and RAPD markers. Biochem Syst Ecol 29: 287-304.
8. Barnard D. and R. D. Xue. 2004. Laboratory evaluation of mosquito repellents against Aedes albopictus, Culex nigripalpus, and Ochlerotatus triseriatus (Diptera: Culicidae). Journal of Medical Entomology 41:726-730.
9. Chen, W., & Viljoen, A. M. (2010). Geraniol—A review of a commercially important fragrance material. South African Journal of Botany, 76(4), 643-651
10. Jesus Olivero-Verbel, ´ a,b* Luz S Nerioa,b and Elena E Stashenko Bioactivity against Tribolium castaneum Herbst (Coleoptera: Tenebrionidae) of Cymbopogon citratus and Eucalyptus citriodora essential oils grown in Colombia.
11. Taylor W G, Schreck C E (1985) Chiral-phase capillary gas chromatography and mosquito repellent activity of some oxazolidine derivatives of (+)− and (−)− citronellol. J Pharm Sci. May;74(5):534-9.
12. Dorman H J 1, Deans S G. (2000) Antimicrobial agents from plants: antibacterial activity of plant volatile oils. J Appl Microbiol. Feb;88(2):308-16.
13. Batish, D. R., Singh, H. P., Setia, N., Kaur, S., & Kohli, R. K. (2006). Chemical composition and phytotoxicity of volatile essential oil from intact and fallen leaves of Eucalyptus citriodora. Zeitschrift fur Naturforschung C-Journal of Biosciences, 61(7-8), 465-471.
14. Dreistadt, S. H. (2004). Pests of landscape trees and shrubs: An integrated pest management guide (Vol. 3359). UCANR Publications.
15. Newete, S. W., Oberprieler, R. G., & Byrne, M. J. (2011). The host range of the Eucalyptus Weevil, Gonipterus “scutellatus” Gyllenhal (Coleoptera: Curculionidae), in South Africa. Annals of forest science, 68(5), 1005-1013.
16. Quang Thu, P., Dell, B., & Burgess, T. I. (2009). Susceptibility of 18 eucalypt species to the gall wasp Leptocybe invasa in the nursery and young plantations in Vietnam. ScienceAsia, 35(2), 113-117.
17. Hammer K A 1, Carson C F, Riley T V. (1999) Antimicrobial activity of essential oils and other plant extracts. J Appl Microbiol. Jun;86(6):985-90.
18. H. J. D. Dorman and S. G. Deans; Antimicrobial agents from plants: antibacterial activity of plant volatile oils. Article first published online: 9 Oct. 2008
19. Regnault-Roger C 1, Vincent C, Arnason J T (2012) Essential oils in insect control: low-risk products in a high-stakes world. Annu Rev Entomol. 2012;57:405-24. doi: 10.1146/annurev-ento-120710-100554. Epub 2011 Sep. 19.
20. Vinod Shanker Dubey, Ritu Bhalla and Rajesh Luthra An overview of the non-mevalonate pathway for terpenoid biosynthesis in plants. J. Biosci. | Vol. 28 | No. 5 | September 2003 | 637-646 |.
21. Lange B M 1, Ahkami A. (2013) Metabolic engineering of plant monoterpenes, sesquiterpenes and diterpenes—current status and future opportunities. Plant Biotechnol J. Feb;11(2):169-96. doi: 10.1111/pbi.12022. Epub 2012 Nov. 21.
22. Lange B. M., Turner G. W. (2013) Terpenoid biosynthesis in secretory glandular trichomes—current status and future opportunities. Plant Biotechnol. J. 11, 2-22.
23. Davidovich-Rikanati R 1, Sitrit Y, Tadmor Y, Iijima Y, Bilenko N, Bar E, Carmona B, Fallik E, Dudai N, Simon J E, Pichersky E, Lewinsohn E. (2007) Enrichment of tomato flavor by diversion of the early plastidial terpenoid pathway. Nat Biotechnol. Aug;25(8):899-901. Epub 2007 Jun. 24.
24. Singh-Sangwan N, Sangwan R S, Luthra R, Thakur R S (1993) Geraniol dehydrogenase: a determinent of essential oil quality in lemongrass. Planta Med 59: 168-170.
25. Sekiwa-Iijima Y, Aizawa Y, Kubota K (2001) Geraniol dehydrogenase activity related to aroma formation in ginger (Zingiber officinale Roscoe). J Agric Food Chem 49: 5902-5906.
26. Tian-Tian Yuan, Qian-Qian Chen, Pei-Ji Zhao, Ying Zeng, Xiao-Zhu Liu, Shan Lu Identification of enzymes responsible for the reduction of geraniol to citronellol 3 Dec. 2011.
27. Iijima Y 1, Wang G, Fridman E, Pichersky E. (2006) Analysis of the enzymatic formation of citral in the glands of sweet basil. Arch Biochem Biophys. Apr 15;448(1-2):141-9. Epub 2005 Aug. 19.
28. Bleeker, P. M., Diergaarde, P. J., Ament, K., Guerra, J., Weidner, M., Schütz, S., . . . & Schuurink, R. C. (2009). The role of specific tomato volatiles in tomato-whitefly interaction. Plant physiology, 151(2), 925-935.
29. Phool Shahzad, Ali Muhammad, Ferhat Mehmood and Muhammad Yasir Chaudhry. Synthesis of 3, 7-Dimethyl-2, 6-Octadienal Acetals from Citral Extracted from Lemon Grass, Cymbopogon citrates L. J Antivir Antiretrovir ISSN: 1948-5964 JAA, an open access journal.
30. Yeon-Suk Lee, Junheon Kim, Sang-Chul Shin, Sang-Gil Lee and Il-Kwon Park Antifungal activity of Myrtaceae essential oils and their components against three phytopathogenic fungi FLAVOUR AND FRAGRANCE JOURNAL Flavour Fragr. J. 2008; 23: 23-28 Published online 11 Jan. 2008 in Wiley InterScience (www(dot)interscience(dot)wiley(dot)com) DOI: 10.1002/ffj.1850.

1. A genetically modified woody plant comprising a heterologous nucleic acid sequence encoding at least one polypeptide selected from the group consisting of Geraniol Synthase (GS), Geraniol Reductase (GR), Geraniol Dehydrogenase (GD) and Citral Reductase (CR).

2. A nucleic acid construct comprising:
a nucleic acid sequence encoding at least two polypeptides selected from the group consisting of Geraniol Synthase (GS), Geraniol Reductase (GR), Geraniol Dehydrogenase (GD) and Citral Reductase (CR) and at least one cis-acting regulatory element for directing expression of said nucleic acid sequence; or
a nucleic acid sequence encoding Geraniol Dehydrogenase (GD) and a cis-acting regulatory element for directing expression of said nucleic acid sequence in a plant cell; or
a nucleic acid sequence encoding Citral Reductase (CR) and a cis-acting regulatory element for directing expression of said nucleic acid sequence in a plant cell, wherein said nucleic acid sequence further comprises a chloroplast leader peptide; or
a nucleic acid sequence encoding Geraniol Reductase (GR) and a cis-acting regulatory element for directing expression of said nucleic acid sequence in a plant cell, wherein said nucleic acid sequence is devoid of a peroxisome C-terminus tri-amino acid signal (SRL) and comprises a chloroplast leader peptide.

3. A nucleic acid construct system comprising at least two nucleic acid constructs expressing at least two polypeptides selected from the group consisting of Geraniol Synthase (GS), Geraniol Reductase (GR), Geraniol Dehydrogenase (GD) and Citral Reductase (CR).

4-6. (canceled)

7. The nucleic acid construct of any one of claim 2, wherein said cis-acting regulatory element comprises a promoter sequence.

8. (canceled)

9. The nucleic acid construct of claim 7, wherein said promoter is selected from the group consisting of Cauliflower mosaic virus (CaMV) 35S promoter, Figwort mosaic virus subgenomic transcript (sgFiMV) promoter and Strawberry vein banding virus (SVBV) promoter.

10-11. (canceled)

12. A genetically modified woody plant comprising the nucleic acid construct of claim 2.

13. The genetically modified woody plant of claim 1 being resistant to pest infection.

14. A pesticidal composition, comprising as an active ingredient the nucleic acid construct of claim 2; and an agriculturally acceptable carrier or diluent.

15. A method of enhancing resistance of a woody plant to pest infection, the method comprising expressing in the woody plant at least one recombinant polypeptide selected from the group consisting of Geraniol Synthase (GS), Geraniol Reductase (GR), Geraniol Dehydrogenase (GD) and Citral Reductase (CR), thereby enhancing the resistance of the woody plant to pest infection.

16. A method of enhancing at least one of geraniol, geranial, neral, citronellol, citronellal and citral oil content of a woody plant, the method comprising expressing in the woody plant at least one recombinant polypeptide selected from the group consisting of Geraniol Synthase (GS), Geraniol Reductase (GR), Geraniol Dehydrogenase (GD) and Citral Reductase (CR), thereby enhancing at least one of geraniol, geranial, neral, citronellol, citronellal and citral oil content of the woody plant.

17. A method of producing oil, the method comprising providing the genetically modified woody plant of claim 1; and extracting the oil from said woody plant, thereby producing oil.

18. The method of claim 17, further comprising purifying a monoterpene fraction from said oil following said extracting.

19. The method of claim 18, wherein said monoterpene fraction comprises at least one of geraniol, geranial, neral, citronellol, citronellal and citral.

20. An oil produced according to the method of claim 17.

21. An eucalyptus oil having an increased content of at least one monoterpene selected from the group consisting of geraniol, geranial, neral, citronellol, citronellal and citral; as compared to a eucalyptus oil of a non-genetically modified eucalyptus.

22. The eucalyptus oil of claim 21 having a reduced content of at least one monoterpene not selected from the group consisting of geraniol, geranial, neral, citronellol, citronellal and citral; as compared to a eucalyptus oil of a non-genetically modified eucalyptus.

23. A method of producing at least one monoterpene selected form the group consisting of geraniol, geranial, neral, citronellol, citronellal and citral, the method comprising providing the genetically modified woody plant of claim 1, and extracting the monoterpene from said woody plant, thereby producing at least one monoterpene selected form the group consisting of geraniol, geranial, neral, citronellol, citronellal and citral.

24. The genetically modified woody plant of claim 1, wherein said at least one polypeptide comprises GS.

25. The genetically modified woody plant of claim 1, wherein said at least one polypeptide comprises at least two polypeptides.

26. The genetically modified plant of claim 25, wherein said at least two polypeptides comprise GS and GR.

27. The genetically modified plant of claim 25, wherein said at least two polypeptides comprise GS and CR.

28. The genetically modified plant of claim 25, wherein said at least two polypeptides comprise GS and GD.

29. The genetically modified plant of claim 25, wherein said at least two polypeptides comprise GS, GR and GD.

30. The genetically modified plant of claim 25, wherein said at least two polypeptides comprise GS, GD and CR.

31. The genetically modified plant claim 25, wherein said at least two polypeptides comprise GS, GR, GD and CR.

32. The genetically modified woody plant of claim 1, wherein said polypeptide further comprises a chloroplast leader peptide.

33. The construct of claim 2, wherein said plant cell is a cell of a woody plant.

34. The genetically modified woody plant of claim 1, wherein said woody plant is Eucalyptus or poplar.

35. The genetically modified plant of claim 13, wherein said pest is selected from the group consisting of Glycaspis brimblecombei, Thaumastocoris peregrinus, Leptocybe invasa and Ophelimus maskelli.

36. The genetically modified woody plant of claim 1, wherein:
said GS nucleic acid sequence comprises SEQ ID NOs: 36-46; or
said GR nucleic acid sequence comprises SEQ ID NOs: 47-57, 82, 84, 86, 88 or 90; or
said GD nucleic acid sequence comprises SEQ ID NOs: 58-63; or
said CR nucleic acid sequence comprises SEQ ID NO: 80.

37-39. (canceled)

40. The genetically modified woody plant, of claim 1, wherein:
said GS amino acid sequence comprises SEQ ID NOs: 1-12; or
said GR amino acid sequence comprises SEQ ID NOs: 13-29, 81, 83, 85, 87 or 89: or
said GD amino acid sequence comprises SEQ ID NOs: 30-35; or
said CR amino acid sequence comprises SEQ ID NO: 79.

41-43. (canceled)


SEQUENCE LISTING (91 sequences)

SEQ ID NO: 1 - protein, 567 aa, Ocimum basilicum
MSCARITVTLPYRSAKTSIQRGITHYPALIRPRFSACTPLASAMPLSSTPLINGDNSQRKNTRQHMEESSSKRREYLLEETTRKLQRNDTESVEKLKLIDNIQQLGIGYYFEDAINAVLRSPFSTGEEDLFTAALRFRLLRHNGIEISPEIFLKFKDERGKFDESDTLGLLSLYEASNLGVAGEEILEEAMEFAEARLRRSLSEPAAPLHGEVAQALDVPRHLRMARLEARRFIEQYGKQSDHDGDLLELAILDYNQVQAQHQSELTEIIRWWKELGLVDKLSFGRDRPLECFLWTVGLLPEPKYSSVRIELAKAISILLVIDDIFDTYGEMDDLILFTDAIRRWDLEAMEGLPEYMKICYMALYNTTNEVCYKVLRDTGRIVLLNLKSTWIDMIEGFMEEAKWFNGGSAPKLEEYIENGVSTAGAYMAFAHIFFLIGEGVTHQNSQLFTQKPYPKVFSAAGRILRLWDDLGTAKEEQERGDLASCVQLFMKEKSLTEEEARSRILEEIKGLWRDLNGELVYNKNLPLSIIKVALNMARASQVVYKHDQDTYFSSVDNYVDALFFTQ

SEQ ID NO: 2 - protein, 567 aa, Ocimum basilicum; identical to SEQ ID NO: 1

SEQ ID NO: 3 - protein, 574 aa, Ocimum basilicum
MSCARITVTLPYRSAKTSIQRGITHCPALLRPRFSACTPLASAVPLSSTPLINGDNSPLKNTHQHVEERSSKRREYLLEETARKLQRNDTESVEKLKLIDNIQRLGIGYYFEDAIDAVLRSPFSAEEEEDLFTAALRFRLLRHNGIQVTPEIFLKFKDERGEFDESDTLGLLSLYEASNLGVTGEEILEEAMEFAEPRLRRSLSELAAPLRSEVAQALDVPRHLRMARLEARRFIEQYGKQSDHDGDLLELAILDYNQVQAQHQSELTEITRWWKQLGLVEKLGFGRDRALECFMWTMGILPHPKYSSSRIESAKAAALLYVIDDIFDTYGKMDELILFTDAIRRWDLEAMEGLPEYMKICYMALYNTTNEICYRVLKDTGRIALPYLKSVWIETIEAYMVEVKWFSGGSAPKLEEYIENGASTVGAYMVLVHLFFLIGEGLTHQNVLFFKQKPYHKPFSAAGRIFRLWDDLGTSQEEEERGDMASSIRLFMKEYKLSTVEEARSCVLEEISRLWKDLNEGLISIKDALPLTIVKVALNIARTSQVVYKHEQHTYMLSVDNYVEALFFTPLLSS

SEQ ID NO: 4 - protein, 363 aa, Olea europaea
  misc_feature (15)..(15): Xaa can be any naturally occurring amino acid
DVFLKFMEKNMKFKXHLAQDTIGLLSLYEASSMGANGEEILSEAKEFTEIHLRQSMPRLVPQLRRQVSSALELPRHLRMARLEARRYIEEYGNESDHDPAVLELARLDYNKVQLQHQMELAEITRWWKQLGLVEKLSFARDRPLECFLWTVGLLPEPKYSSCRIELAKTIAILLVIDDIFDTYGKMEELVLFTEAIQRWDLDELETLPPYMRICYMALYNTTNEICYKILKEYGFCVLPYLKSTWIDMIEGFMVEANWFNGGHGPNLEEYIENGVSTAGAYMALVHLFFLIGEGVTNENIAKLLRKPYPKLFSAAGRILRLWDDLGTAKEEEERGDLASCMQILMREKNIDCENEGRNYILKA

SEQ ID NO: 5 - protein, 363 aa, Olea europaea
  misc_feature (25)..(25): Xaa can be any naturally occurring amino acid
  misc_feature (50)..(50): Xaa can be any naturally occurring amino acid
  misc_feature (58)..(58): Xaa can be any naturally occurring amino acid
  misc_feature (60)..(60): Xaa can be any naturally occurring amino acid
DVFLKFMEKNMKFKEHLAQDTIGLXSLYEASSMGANGEEILSEAKEFTEXHLRQSMPXLXPQLRRQVSSALELPRHLRMARLEARRYIEEYGNESDHDPAVLELARLDYNKVQLQHQMELAEITRWWKQLGLVEKLSFARDRPLECFLWTVGLLPEPKYSSCRIELAKTIAILLVIDDIFDTYGKMEELVLFTEAIQRWDLDELETLPPYMRICYMALYNTTNEICYKILKEYGFCVLPYLKSTWIDMIEGFMVEANWFNGGHGPNLEEYIENGVSTAGAYMALVHLFFLIGEGVTNENIAKLLRKPYPKLFSAAGRILRLWDDLGTAKEEEERGDLASCMQILMREKNIDCENEGRNYILKA

SEQ ID NO: 6 - protein, 583 aa, Olea europaea
MDCTMTSISLFSQSSNGISGTARSPFQWPINHRFSSGQRDFICKSLPVSSPSATPLIPAENGAMYNYIRQPVIVTPEVDDGTKHSELVERTRRELQRSTKPVETLKLIDNLQRLGIAYYFEDDINAILDQFSDGLPDEDLFTTALCFRLLRDQRLQTGSDVFLKFMEKNMKFKEHLAQDTIGLVSLYEASSMGANGEEILSEAKEFTEMHLRQSMPQLAPQLRRQVSSALELPRHLRMARLEARRYIEEYGNESDHDPALLELARLDYNKVQLQHQMELAEITRWWKQLGLVEKLSFARDRPLECFLWTVGLLPEPKYSSCRIELAKTIAILLVIDDIFDTYGKMEELVLFTEAIQRWDLDELETLPPYMRICYMALYNTTNEICYKILKEYGFCVLPYLKSTWIDMIEGFMVEANWFNGGHGPNLEEYIENGVSTAGAYMALVHLFFLIGEGVTNENIAKLLRKPYPKLFSAAGRILRLWDDLGTAKEEEERGDLASCMQILMREKNIDCENEGRNYILKAINGLWKDLNDELISPNAMPLAITKVALNMARAFEVVYKHEEDSYFSSVDNYVQALFFTPIN

SEQ ID NO: 7 - protein, 584 aa, Phyla dulcis
MASARSTISLSSQSSHHGFSKNSFPWQLRHSRFVMGSRARTCACMSSSVSLPTATTSSSVITGNDALLKYIRQPMVIPLKEKEGTKRREYLLEKTARELQGTTEAAEKLKFIDTIQRLGISCYFEDEINGILQAELSDTDQLEDGLFTTALRFRLLRHYGYQIAPDVFLKFTDQNGKFKESLADDTQGLVSLYEASYMGANGENILEEAMKFTKTHLQGRQHAMREVAEALELPRHLRMARLEARRYIEQYGTMIGHDKDLLELVILDYNNVQAQHQAELAEIARWWKELGLVDKLTFARDRPLECFLWTVGLLPEPKYSACRIELAKTIAILLVIDDIFDTYGKMEELALFTEAIRRWDLEAMETLPEYMKICYMALYNTTNEICYKVLKKNGWSVLPYLRYTWMDMIEGFMVEAKWFNGGSAPNLEEYIENGVSTAGAYMALVHLFFLIGEGVSAQNAQILLKKPYPKLFSAAGRILRLWDDLGTAKEEEGRGDLASSIRLFMKEKNLTTEEEGRNGIQEEIYSLWKDLNGELISKGRMPLAIIKVALNMARASQVVYKHDEDSYFSCVDNYVEALFFTPLL

SEQ ID NO: 8 - protein, 589 aa, Catharanthus roseus
MAATISNLSFLAKSRALSRPSSSSLSWLERPKTSSTICMSMPSSSSSSSSSSMSLPLATPLIKDNESLIKFLRQPLVLPHEVDDSTKRRELLERTRKELELNAEKPLEALKMIDIIQRLGLSYHFEDDINSILTGFSNISSQTHEDLLTASLCFRLLRHNGHKINPDIFQKFMDNNGKFKDSLKDDTLGMLSLYEASYLGANGEEILMEAQEFTKTHLKNSLPAMAPSLSKKVSQALEQPRHRRMLRLEARRFIEEYGAENDHNPDLLELAKLDYNKVQSLHQMELSEITRWWKQLGLVDKLTFARDRPLECFLWTVGLLPEPKYSGCRIELAKTIAILLVIDDIFDTHGTLDELLLFTNAIKRWDLEAMEDLPEYMRICYMALYNTTNEICYKVLKENGWSVLPYLKATWIDMIEGFMVEAEWFNSDYVPNMEEYVENGVRTAGSYMALVHLFFLIGQGVTEDNVKLLIKPYPKLFSSSGRILRLWDDLGTAKEEQERGDLASSIQLFMREKEIKSEEEGRKGILEIIENLWKELNGELVYREEMPLAIIKTAFNMARASQVVYQHEEDTYFSSVDNYVKALFFTPCF

SEQ ID NO: 9 - protein, 586 aa, Picrorhiza kurrooa
MGCIRSTTHTSTNYGINSRLGLSFPVPIIYRPAGASRNISIYMSSSAAPNVTQVVVGANDALLQYTHQPMLIPSEVDADTERRDYLLEKARQELSRTTKPTDKLKLIDMIQRLGIGFYLEDDINAILRTQFSVNVIDEEDLSATGLRFRMLRQNSFPISSDIFKRFQDQNEKFDETLTNDTLGVIELVRSIIHGEPKGEEILEEAMEFSKTRLKRSLSSLPAQIKADVGQALELPTHLRMARLEARRFIEQYATQSDHNPILLELATLDYNHVQSQHQMELVEITRWWKQLGLVDQLSFARDRPLECFLWTVGLLPEPKYSSCRIELAKTIAVLLVIDDIFDTYGKMDDLILFTDAIQRWDLGAMESLPEYMKICYMALYNLTNEICYKVLKENGWTVLPYLKSSWIDMIEGFMVEAKWFNGGCAPNLEEYVENGVSTAGSYMALVHLFFLIGEGVTEQNAQLLMKKPYPKAFSSAGRILRLWDDLGTAKEEQERGDLASSINLFMKERNLATEEEGRECILEDIYGYWKDLNGELVSQKEMPLAIIKVALNMARASQVVYKHEEDSYFSNVDNYVQALLFTPLII

SEQ ID NO: 10 - protein, 594 aa, Valeriana officinalis
  misc_feature (228)..(228): Xaa can be any naturally occurring amino acid
MITSSSSVRSLCCPKTSIISGKLLPSLLLTNVINVSNGTSSRACVSMSSLPVSKSTASSIAAPLVRDNGSALNFFPQAPQVEIDESSRIMELVEATRRTLRNESSDSTEKMRLIDSLQRLGLNHHFEQDIKEMLQDFANEHKNTNQDLFTTSLRFRLLRHNGFNVTPDVFNKFTEENGKFKESLGEDTIGILSLYEASYLGGKGEEILSEAMKFSESKLRESSGHVAXHIRRQIFQSLELPRHLRMARLESRRYIEEDYSNEIGADSSLLELAKLDFNSVQALHQMELTEISRWWKQLGLSDKLPFARDRPLECFLWTVGLLPEPKYSGCRIELAKTIAVLLVIDDIFDTYGSYDQLILFTNAIRRWDLDAMDELPEYMKICYMALYNTTNEICYKVLKENGWSVLPYLERTWIDMVEGFMLEAKWLNSGEQPNLEAYIENGVTTAGSYMALVHLFFLIGDGVNDENVKLLLDPYPKLFSSAGRILRLWDDLGTAKEEQERGDVSSSIQLYMKEKNVRSESEGREGIVEIIYNLWKDMNGELIGSNALPQAIIETSFNMARTSQVVYQHEDDTYFSSVDNYVQSLFFTPVSVSV

SEQ ID NO: 11 - protein, 591 aa, Vitis vinifera
MSRFVTMPSHVLPSSFVAPSLQVSSSPCSWRTRPSPCTSCHLSPSSSSKPLLGSHDYSLLKSLTLSPHAVNSEADSSTRRMKEVKERTWEAFYRAWDSRAAMEMVDTVERLGLSYHFEDEINALLQRFCDWNASEDLFTTALRFRLLRQNGFPTHSDVFGKFMDKNGKFKESLTEDIWGMLSLHEASHLGAKNEEVLAEAKEFTRIHLIQSMPHMEPHFSSHVGRALELPRHLRMVRLEARNYIGEYSRESNPNLAFLELAKLDFDMVQSLHQKELAEIVRWWKQLGLVDKLDFARDRPMECFLWTVGIFPDPRHSSCRIELTKAIAILLVIDDIYDSYGSLDELALFTDAVKRWDLGAMDQLPEYMKICYMALYNTTNDIAYRILKEHGWSVIEDLKRTWMDIFGAFLAEAYCFKGGHVPSLEEYLTNAVTTGGTYMALVHAFFLMGQGVTRENMAMLKPYPNIFSCSGKILRLWDDLGTAREEQERGDNASSIECYKREREMDTVLEDEACRKHIRQMIQSLWVELNGELVASSALPLSIIKAAFNLSRTAQVIYQHGDDNKTSSVEDHVQALLFRPVSSNGHAQITMH

SEQ ID NO: 12 - protein, 595 aa, Vitis vinifera
MAFNMSRFVTMPSHVLPSSFVAPSLQVSSSPCSWRTRPSPCTSCHLSPSSSSKPLLGSHDYSLLKSLTLSPHAVNSEADSSTRRMKEVKERTWEAFYRAWDSRAAMEMVDTVERLGLSYHFEDEINALLQRFCDWNASEDLFTTALRFRLLRQNGFPTHSDVFGKFMDKNGKFKESLTEDIRGMLSLHEASHLGAKNEEVLAEAKEFTRIHLIQSMPHMEPHFSSHVGRALELPRHLRMVRLEARNYIGEYSRESNPNLAFLELAKLDFDMVQSLHQKELAEILRWWKQLGLVDKLDFARDRPMECFLWTVGIFPDPRHSSCRIELTKAIAILLVIDDIYDSYGSLDELALFTDAVKRWDLGAMDQLPEYMKICYMALYNTTNDIAYRILKEHGWSVIEHLKRTWMDIFGAFLAEAYCFKGGHVPSLEEYLTNAVTTGGTYMALVHAFFLMGQGVTRENMAMLKPYPNIFSCSGKILRLWDDLGTAREEQERGDNASSIECYKREREMDTVLEDEACRKHIRQMIQSLWVELNGELVASSALPLSIIKAAFNLSRTAQVIYQHGDDNKTSSVEDHVQALLFRPVSSNGHAQITMH

SEQ ID NO: 13 - protein, 400 aa, Saccharomyces cerevisiae
MPFVKDFKPQALGDTNLFKPIKIGNNELLHRAVIPPLTRMRAQHPGNIPNRDWAVEYYAQRAQRPGTLIITEGTFPSPQSGGYDNAPGIWSEEQIKEWTKIFKAIHENKSFAWVQLWVLGWAAFPDTLARDGLRYDSASDNVYMNAEQEEKAKKANNPQHSITKDEIKQYVKEYVQAAKNSIAAGADGVEIHSANGYLLNQFLDPHSNNRTDEYGGSIENRARFTLEVVDAVVDAIGPEKVGLRLSPYGVFNSMSGGAETGIVAQYAYVLGELERRAKAGKRLAFVHLVEPRVTNPFLTEGEGEYNGGSNKFAYSIWKGPIIRAGNFALHPEVVREEVKDPRTLIGYGRFFISNPDLVDRLEKGLPLNKYDRDTFYKMSAEGYIDYPTYEEALKLGWDKN

SEQ ID NO: 14 - protein, 400 aa, Saccharomyces cerevisiae; identical to SEQ ID NO: 13

SEQ ID NO: 15 - protein, 400 aa, Saccharomyces cerevisiae
MPFVKDFKPQALGDTNLFKPIKIGNNELLHRAVIPPLTRMRAQHPGNIPNRDWAVEYYAQRAQRPGTLIITEGTFPSPQSGGYDNAPGIWSEEQIKEWTKIFKAIHEKKSFAWVQLWVLGWAAFPDTLARDGLRYDSASDNVYMNAEQEEKAKKANNPQHSITKDEIKQYVKEYVQAAKNSIAAGADGVEIHSANGYLLNQFLDPHSNNRTDEYGGSIENRARFTLEVVDAVVDAIGPEKVGLRLSPYGVFNSMSGGAETGIVAQYAYVLGELERRAKAGKRLAFVHLVEPRVTNPFLTEGEGEYNGGSNEFAYSIWKGPIIRAGNFALHPEVVREEVKDPRTLIGYGRFFISNPDLVDRLEKGLPLNKYDRDTFYKMSAEGYIDYPTYEEALKLGWDKN

SEQ ID NO: 16 - protein, 400 aa, Saccharomyces cerevisiae
MPFVKDFKPQALGDTNLFKPIKIGNNELLHRAVIPPLTRMRAQHPGNIPNRDWAVEYYAQRAQRPGTLIITEGTFPSPQSGGYDNAPGIRSEEQIKEWTKIFKAIHENKSFAWVQLWVLGWAAFPDTLARDGLRYDSASDNVYMNAEQEEKAKKANNPQHSITKDEIKQYVKEYVQAAKNSIAAGADGVEIHSANGYLLNQFLDPHSNNRTDEYGGSIENRARFTLEVVDAVVDAIGPEKVGLRLSPYGVFNSMSGGAETGIVAQYAYVLGELERRAKAGKRLAFVHLVEPRVTNPFLTEGEGEYNGGSNKFAYSIWKGPIIRAGNFALHPEVVREEVKDPRTLIGYGRFFISNPDLVDRLEKGLPLNKYDRDTFYKMSAEGYIDYPTYEEALKLGWDKN

SEQ ID NO: 17 - protein, 400 aa, Saccharomyces cerevisiae
MPFVKDFKPQALGDTNLFKPIKIGNNELLHRAVIPPLTRMRAQHPGNIPNRDWAVEYYSQRAQRPGTLIITEGTFPSPQSGGYDNAPGIWSEEQIKEWTKIFKAIHEKKSFAWVQLWVLGWAAFPDTLARDGLRYDSASDNVYMNAEQEEKAKKANNPQHSITKDEIKQYVKEYVQAAKNSIAAGADGVEIHSANGYLLNQFLDPHSNNRTDEYGGSIENRARFTLEVVDAVVDAIGPEKVGLRLSPYGVFNSMSGGAETGIVAQYAYVLGELERRAKAGKRLAFVHLVEPRVTNPFLTEGEGEYNGGSNEFAYSIWKGPIIRAGNFALHPEVVREEMKDPRTLIGYGRFFISNPDLVDRLEKGLPLNKYDRDTFYKMSAEGYIDYPTYEEALKLGWDKN

SEQ ID NO: 18 - protein, 400 aa, Saccharomyces cerevisiae
MPFVKDFKPQALGDTNLFKPIKIGNNELLHRAVIPPLTRMRAQHPGNIPNRDWAVEYYAQRAQRPGTLIITEGTFPSPQSGGYDNAPGIWSEEQIKEWTKIFKAIHEKKSFAWVQLWVLGWAAFPDTLARDGLRYDSASDNVYMNAEQEEKAKKANNPQHSITKDEIKQYVKEYVQAAKNSIAAGADGVEIHSANGYLLNQFLDPHSNNRTDEYGGSIENRARFTLEVVDAVVDAIGPEKVGLRLSPYGVFNSMSGGAETGIVAQYAYVLGELERRAKAGKRLAFVHLVEPRVTNPLLTEGEGEYNGGSNEFAYSIWKGPIIRAGNFALHPEVVREEVKDPRTLIGYGRFFISNPDLVDRLEKGLPLNKYDRDTFYKMSAEGYIDYPTYEEALKLGWDKN

SEQ ID NO: 19 - protein, 400 aa, Saccharomyces cerevisiae
MPFVKDFKPQALGDTNLFKPIKIGNNELLHRAVIPPLTRMRAQHPGNIPNRDWAVEYYSQRAQRPGTLIITEGTFPSPQSGGYDNAPGIWSEEQIKEWTKIFKAIHEKKSFAWVQLWVLGWAAFPDTLARDGLRYDSASDNVYMNAEQEEKAKKANNPQHSITKDEIKQYVKEYVQAAKNSIAAGADGVEIHGANGYLLNQFLDPHSNNRTDEYGGSIENRARFTLEVVDAVVDAIGPEKVGLRLSPYGVFNSMSGGAETGIVAQYAYVLGELERRAKAGKRLAFVHLVEPRVTNPFLTEGEGEYNGGSNEFAYSIWKGPIIRAGNFALHPEVVREEVKDPRTLIGYGRFFISNPDLVDRLEKGLPLNKYDRDTFYKMSAEGYIDYPTYEEALKLGWDKN

SEQ ID NO: 20 - protein, 419 aa, Saccharomyces cerevisiae
MPFVKDFKPQALGDTNLFKPIKIGNNELLHRAVIPPLTRMRAQHPGNIPNRDWAVEYYAQRAQRPGTLIITEGTFPSPQSGGYDNAPGIWSEEQIKEWTKIFKAIHEKKSFAWVQLWVLGWAAFPDTLARDGLRYDSASDNVYMNAEQEEKAKKANNPQHSITKDEIKQYVKEYVQAAKNSIAAGADGVEIHSANGYLLNQFLDPHSNNRTDEYGGSIENRARFTLEVVDAVVDAIGPEKVGLRLSPYGVFNSMSGGAETGIVAQYAYVLGELERRAKAGKRLAFVHLVEPRVTNPFLTEGEGEYNGGSNEFAYSIWKGPIIRAGNFALHPEVVREEVKDPRTLIGYGRFFISNPDLVDRLEKGLPLNKYDRDTFYKMSAEGYIDYPTYEEALKLGWEQKLIVLTVLCSTISFSSNLYN

SEQ ID NO: 21 - protein, 400 aa, Saccharomyces cerevisiae
MSFVKDFKPQALGDTNLFKPIKIGNNELLHRAVIPPLTRMRAQHPGNIPNRDWAVEYYAQRAQRPGTLIITEGTFPSPQSGGYDNAPGIWSEEQIKEWTKIFKAIHEKKSFAWVQLWVLGWAAFPDTLARDGLRYDSASDNVYMNAEQEEKAKKANNPQHSITKDEIKQYVKEYVQAAKNSIAAGADGVEIHSANGYLLNQFLDPHSNNRTDEYGGSIENRARFTLEVVDAVVDAIGPEKVGLRLSPYGVFNSMSGGAETGIVAQYAYVLGELERRAKAGKRLAFVHLVEPRVTNPFLTEGEGEYNGGSNEFAYSIWKGPIIRAGNFALHPEVVREEVKDPRTLIGYGRFFISNPDLVDRLEKGLPLNKYDRDTFYKMSAEGYIDYPTYEEALKLGWDKH

SEQ ID NO: 22 - protein, 400 aa, Candida sake
MSFVKDFKPQALGDTNLFKPIKIGNNELLHRAVIPPLTRMRAQHPGNIPNRDWAVEYYAQRAQRPGTLIITEGTFPSPQSGGYDNAPGIWSEEQIKEWTKIFKAIHENKSFAWVQLWVLGWAAFPDTLARDGLRYDSASDNVYMNAEQEEKAKKANNPQHSITKDEIKQYVKEYVQAAKNSIAAGADGVEIHSANGYLLNQFLDPHSNNRTDEYGGSIENRARFTLEVVDAVVDAIGPEKVGLRLSPYGVFNSMSGGAETGIVAQYAYVLGELERRAKAGKRLAFVHLVEPRVTNPFLTEGEGEYNGGSNKFAYSIWKGPIIRAGNFALHPEVVREEVKDPRTLIGYGRFFISNPDLVDRLEKGLPLNKYDRDTFYKMSAEGYIDYPTYEEALKLGWDKK

SEQ ID NO: 23 - protein, 400 aa, Kazachstania exigua
MPFVKDFKPQALGDTNLFKPIKIGNNELLHRAVIPPLTRMRAQHPGNIPNRDWAVEYYAQRAQRPGTLIITEGTFPSPQSGGYDNAPGIWSEEQIKEWTKIFKAIHEKKSFAWVQLWVLGWAAFPDTLARDGLRYDSASDNVYMNAEQEEKAKKANNPQHSITKDEIKQYVKEYVQAAKNSIAAGADGVEIHSANGYLLNQFLDPHSNNRTDEYGGSIENRARFTLEVVDAVVDAIGPEKVGLRLSPYGVFNSMSGGAETGIVAQYAYVLGELERRAKAGKRLAFVHLVEPRVTNPFLTEGEGEYNGGSNEFAYSIWKGPIIRAGNFALHPEVVREEVKDPRTLIGYGRFFISNPDLVDRLEKGLPLNKYDRDTFYKMSAEGYIDYPTYEEALKLGWDKH

SEQ ID NO: 24 - protein, 400 aa, Naumovozyma castellii
MSFVKDFKPQALGDTNLFKPTKIGNNELLHRAVIPPLTRMRAQHPGNIPNRDWAVEYYAQRAQRPGTLIITEGTFPSPQSGGYDNAPGIWSEEQIKEWTKIFKAIHENKSFAWVQLWVLGWAAFPDTLARDGLRYDSASDNVYMNAEQEEKAKKANNPQHSITKDEIKQYVKEYVQAAKNSIAAGADGVEIHSANGYLLNQFLDPHSNNRTDEYGGSIENRARFTLEVVDAVVDAIGPEKVGLRLSPYGVFNSMSGGAETGIVAQYAYVLGELERRAKAGKRLAFVHLVEPRVTNPFLTEGEGEYNGGSNKFAYSIWKGPIIRAGNFALHPEVVREEVKDPRTLIGYGRFFISNPDLVDRLEKGLPLNKYDRDTFYKMSAEGYIDYPTYEEALKLGWDKQ

SEQ ID NO: 25 - protein, 400 aa, Kazachstania spencerorum
MSFVKDFKPQALGDTNLFKPIKIGNNELLHRAVIPPLTRMRAQHPGNIPNRDWAVEYYAQRAQRPGTLIITEGTFPSPQSGGYDNAPGIWSEEQIKEWTKIFKAIHEKKSFAWVQLWVLGWAAFPDTFARDGLRYDSASDNVYMNAEQEEKAKKANNPQHSITKDEIKQYAKEYVQAAKNSIAAGADGVEIHSANGYLLNQFLDPHSNNRTDEYGGSIENRARFTLEVVDAVVDAIGPEKVGLRLSPYGVFNSMSGGAETGIVAQYAYVLGELERRAKAGKRLAFVHLVEPRVTNPFLTEGEGEYNGGSNEFAYSIWKGPIIRAGNFALHPEVVREEVKDPRTLIGYGRFFISNPDLVDRLEKGLPLNKYDRDTFYKMSAEGYIDYPTYEEALKLGWDKQ

SEQ ID NO: 26 - protein, 400 aa, Kazachstania solicola
MSFVKDFKPQALGDTNLFKPIKIGNNELLHRAVIPPLTRMRAQHPGNIPNRDWAVEYYAQRAQRPGTLIITEGTFPSPQSGGYDNAPGIWSEEQIKEWTKIFKAIHEKKSFAWIQLWVLGWAAFPDTFARDGLRYDSASDNVYMNAEQEEKAKKANNPQHSITKDEIKQYVKEYVQAAKNSIAAGADGVEIHSANGYLLNQFLDPHSNNGTDEYGGSIENRARFTLEVVDAVVDAIGPEKVGLRLSPYGVFNSMSGGAETGIVAQYAYVLGELERRAKAGKRLAFVHLVEPRVTNPFLTEGEGEYNGGSNEFAYSIWKGPIIRAGNFALHPEVVREEVKDPRTLIGYGRFFISNPDLVDRLEKGLPLNKYDRDTFYKMSAEGYIDYPTYEEALKLGWDKQ

SEQ ID NO: 27 - protein, 400 aa, Nakaseomyces bacillisporus
MSFVKDFKPQALGDTNLFKPIKIGNNELLHRAVIPPLTRMRAQHPGNIPNRDWAVEYYAQRAQRPGTLIITEGTFPSPQSGGYDNAPGIWSEEQIKEWTKIFKAIHEKKSFAWVQLWVLGWAAFPDTFARDGLRYDSASDNVYMNAEQEEKAKKANNPQHSITKDEIKQYVKEYVQAAKNSIAAGADGVEIHSANGYLLNQFLDPHSNNRTDEYGGSIENRARFTLEVVDAVVGAIGPEKVGLRLSPYGVFNSMSGGAETGIVAQYAYVLGELERRAKVGKRLAFVHLVEPRVTNPFLTEGEGEYNGGSNEFAYSIWKGPIIRAGNFALHPEVVREEVKDPRTLIGYGRFFISNPDLVDRLEKGLPLNKYDRDTFYKMSAEGYIDYPTYEEALKLGWDKQ

SEQ ID NO: 28 - protein, 400 aa, Saccharomyces cerevisiae x Saccharomyces kudriavzevii VIN7
MSFVKDFKPQALNDTNLFKPIKIGNNELLHRAVIPPLTRMRAQHPGNVPNKDWAVEYYTQRAQRPGTLIITEGTFISPQSGGYDNAPGIWSEEQMKEWTKIFKAIHEKKSFAWVQLWVLGWAAFPDTLARDGLRYDSASDNVYMNAEQEEKAKKANNPQHSLTKDEIKQYIKEYVQAAKNSIAAGADGVEIHSANGYLLNQFLDPHSNNRTDEYGGSIENRARFTLEVVDAIVEAIGQEKVGLRLSPYGVFNSMSGGAETGIVAQYAYVLGELERRANAGKRLAFVHLVEPRVTNPFLAEGEGAYDGGSNQFVYSIWKGAIIRAGNYALHPEVVREEVKDPRTLIGYGRFFISNPDLVDRLEKGLPLNKYDRTTFYSMSAKGYIDYPTYEEALKLGWDKN

SEQ ID NO: 29 - protein, 400 aa, Saccharomyces kudriavzevii IFO 1802
MSFVKDFKPQALNDTNLFKPIKIGNNELLHRAVIPPLTRMRAQHPGNVPNKDWAVEYYTQRAQRPGTLIITEGTFISPQSGGYDNAPGIWSEEQMKEWTKIFKAIHEKKSFAWVQLWVLGWAAFPDTLARDGLRYDSASDNVYMSAEQEEKAKKANNPQHSLTKDEIKQYIKEYVQAAKNSIAAGADGVEIHSANGYLLNQFLDPHSNNRTDEYGGSIENRARFTLEVVDAIVEAIGQEKVGLRLSPYGVFNSMSGGAETGIVAQYAYVLGELERRANAGKRLAFVHLVEPRVTNPFLAEGEGAYDGGSNQFVYSIWKGAIIRAGNYALHPEVVREEVKDPRTLIGYGRFFISNPDLVDRLEKGLPLNKYDRTTFYSMSAKGYIDYPTYEEALKLGWDKN

SEQ ID NO: 30 - protein, 360 aa, Ocimum basilicum
MAKSPETEHPVKALGWAATDNSGTFSPFNFSRRATGERDVQFRVLYCGVCHSDLHMVKNEWGVTHYPIVPGHEIVGIVTEVGSKVEKVKIGDKVGVGVLVGSCRQCDQCSNDLENYCYKQILTYGAPYLDGTIARGGYSDIMVADEHFIIRWPENFPLDAGAPLLCAGITTYSPLKYFGLDKPGLRVGVNGLGGLGHVAVKFAKAFGTKVTVISTSLSKKEEAMQHLGVDEFVVSTDPQQMQAAVGTLDGIIDTVSAPHPIVPLLSLLKPHGKLIVVGLPDKPLQLPVFPLIQGRRTIAGSGIGGLKETQEMIDFAAKNNIVADVEVIPIDYINTAMDRLLKSDVKYRFVIDVEKSLKPQ

SEQ ID NO: 31 - protein, 360 aa, Ocimum basilicum; identical to SEQ ID NO: 30

SEQ ID NO: 32 - protein, 354 aa, Ocimum americanum
MAKSPETEHPVKALGWAATDNSGTFSPFNFSRRATGERDVQFRVLYCGVCHSDLHMVKNEWGVTLYPIVPGHEIVGIVTEVGSKVEKVKVGDKVGVGVLVGSCRQCDQCSNDLENYCYKQILTYGAPYLDGTITRGGYSDTDEHFIIRWPENFPLDAGAPLLCAGITTYSPLKYFGLDKPGLRVGVNGLGGLGHVAVKFAKAFGTKVTVISTSLAKKEEAIQHLGVDEFVVSTDPQQMQAAVGTLDGIIDTVSAPHPVVPLLSLLKPHGKLIVVGLPDKPLQLPAFPLIQGRKTVAGSGIGGLKETQEMIDFAAKNNIVADVEVIPIDYINTAMDRLDVKYRFVIDVEKSLKPQ

SEQ ID NO: 33 - protein, 360 aa, Perilla setoyensis
MAKTPETEHPLKAFGWAARDNSGTLSPFNFSRRDTGERDVQFKVLYCGVCHSDLHMVKNEWGVTHYPVVPGHEIVGVVTEVGSKVEKVKAGDKVGVGVIVGSCRQCDQCSNDLENYCSKQILTYGAPYIDGTITRGGYSNIMVADEHFIIRWPENLPLDAGAPLLCAGITTYSPLRYYGLDKPGLSVGVNGLGGLGHVAVKFAKAFGTKVTVISTSLGKKKEAIEHLGVDEFLVSSDPQQMQAAVGTLDGIIDTVSAEHPLAPLMSLLKPHGKLIVVGAPQKPLQLHAFSLIQGRKTVAGSGIGGMKETQEMIDFAAKKNIIPDVEVIPIDYINTAMDRLLKSDVKYRFVIDVNKSFNAH

SEQ ID NO: 34 - protein, 360 aa, Perilla setoyensis
MAKTPETEHPVKAFGWAARDNSGTLSPFNFSRRDTGERDVQFKVLYCGVCHSGLHMVKNEWGVTHYPVVPGHEIVGVVTEVGSKVEKVKAGDKVGVGVIVGSCRQCDQCSNDLENYCSKQILTYGAPYIDGTITRGGYSNIMVADEHFIIRWPENLPLDAGAPLLCAGITTYSPLRYYGLDKPGLSVGVNGLGGLGHVAVKFAKAFGTKVTVISTSLGKKKEAIEHLGVDEFLVSSDPQQMQAAVGTLDGIIDTVSAEHPLVPLMSLLKPHGKLIVVGAPEKPLQLHAFSLIQGRKTVAGSAIGGMKETQEMIDFAAKKNIIPDVEVIPIDYINTAMDRLLKSDVKYRFVIDVNKSFNAH

SEQ ID NO: 35 - protein, 360 aa, Perilla setoyensis
MAKTPETEHPVKAFGWAARDNSGTLSPFNFSRRDTGERDVQFKVLYCGVCHSDLHMVKNERGVTHYPVVPGHEIVGVVTEVGSKVEKVKAGDKVGVGVIVGSCRQCDQCSNDLENYCSKQILTYGAPYIDGTITRGGYSNIMVADEHFIIRWPENLPLDAGAPLLCAGITTYSPLRYYGLDKPGLSVGVNGLGGLGHVAVKFAKAFGTKVTVISTSLGKKKEAIEHLGVDEFLVSSDPQQMQAAVGTLDGIIDTVSAEHPLVPLMSLLKPHGKLIVVGAPEKPLQLHAFSLIQGRKTVAGSAIGGMKETQEMIDFAAKKNIIPDVEVIPIDYINTAMDRLLKSDVKYRFVIDVNKSFNAH

SEQ ID NO: 36 - DNA, 1704 bp, Artificial sequence
  Single strand DNA oligonucleotide
atgagctgcgctcgaattaccgttaccttgccttatcgtagcgcaaagacctctatccaacgtggcataactcattatcctgctttgattcgtcctagattcagtgcctgcacacctctcgcctcagctatgcctttgtcatctacacctctgattaatggtgataactcacagcgcaagaataccagacagcatatggaggaaagttcaagtaagagacgcgaatacctcttggaagagacaactagaaaactgcaacgcaacgatacagaatccgtggaaaagctcaaattgatcgacaatattcagcagctcggtatcggctactatttcgaggatgcaatcaacgctgtgcttcgaagcccattttctaccggagaggaagacttgttcactgcagctttgcgattccgtttgctgcgtcacaatggaattgaaattagcccagagatcttcctgaagttcaaagacgagaggggaaaattcgacgaatctgatacacttggtttgctgagtctctacgaggcttcaaacctcggagtcgcaggagaggaaatcctcgaagaggctatggagttcgcagaagctagacttcgaagatctctgagcgagcctgccgcacctctccatggagaagtggcacaagcactggatgttcctagacaccttagaatggctcgattggaagcaaggcgattcatagagcagtatgggaaacaaagcgatcatgacggagatttgcttgagctggctatccttgattataatcaggtccaagcccagcaccagagcgagttgacagagattattcgttggtggaaggagcttggactcgtggacaaattgtctttcgggcgagatcgaccccttgagtgctttctctggacagtcggactgcttccagagccaaaatactcttcagttaggatcgaattggccaaggcaattagtattctgctggtcatagacgacatctttgatacttacggtgaaatggatgatctgattctctttactgacgctattcgaagatgggaccttgaagcaatggagggactccctgagtatatgaagatatgttatatggctctctacaatactacaaacgaagtttgctacaaagtcttgcgtgacacaggcaggatagtccttctgaatcttaagtccacctggattgatatgatagaaggcttcatggaagaggccaaatggtttaatggtggctccgccccaaagttggaggagtatattgagaacggagtttccaccgccggggcctacatggcctttgctcatattttctttctcattggcgagggagtgacccaccagaatagccaactctttactcaaaagccctatcccaaagtgttctctgcagctggacgtattcttcgactttgggatgatctgggcactgccaaagaggaacaagagagaggggatcttgcaagctgcgtccaactgtttatgaaagagaagtctctgacagaagaagaagccaggagcagaatcctggaagaaatcaaagggctttggcgagatttgaatggagagctggtctacaacaaaaacctgcctctctccataatcaaggtcgcattgaacatggctcgcgctagtcaagttgtctataagcacgatcaagacacctacttttcatctgtcgataattatgtggacgctcttttctttacccagtaa

SEQ ID NO: 37 - DNA, 1704 bp, Ocimum basilicum
atgtcttgtgcacggatcaccgtaacattgccgtatcgctccgcaaaaacatcaattcaacggggaattacgcattaccccgcccttatacgcccacgcttctctgcttgcacgcctttggcatcggcgatgcctctaagttcaactcctctcatcaacggggataactctcagcgtaaaaacacacgtcaacacatggaggagagcagcagcaagaggagagaatatctgctggaggaaacgacgcgaaaactgcagagaaacgacaccgaatcggtggagaaactcaagcttatcgacaacatccaacagttgggaatcggctactattttgaggacgccatcaacgccgtactccgctcgcctttctccaccggagaagaagacctcttcaccgctgctctgcgcttccgcttgctccgccacaacggcatcgaaatcagccctgaaatattcctaaaattcaaggacgagaggggaaaattcgacgaatcggacacgctagggttactgagcttgtacgaagcgtcaaatttgggggttgcaggagaagaaatattggaggaggctatggagtttgcggaggctcgcctgagacggtcgctgtcagagccggcggcgccgcttcatggtgaggtggcgcaagcgctagatgtgccgaggcatctgagaatggcgaggttggaagcgagacgattcatcgagcagtatggtaaacagagcgatcatgatggagatcttttggagctggcaattttggattataatcaagttcaggctcaacaccaatccgaactcactgaaataatcaggtggtggaaggagctcggtttggtggataagttgagttttgggcgagacagaccattggagtgctttttgtggaccgtggggctcctcccagagcccaagtattcgagcgttagaatagagttggcgaaagccatctctattctcttagtgatcgatgatattttcgatacctatggagagatggatgacctcatcctcttcaccgatgcaattcgaagatgggatcttgaagcaatggaggggctccctgagtacatgaaaatatgctacatggcgttgtacaataccaccaatgaagtatgctacaaagtgctcagggatactggacggattgtcctccttaacctcaaatctacgtggatagacatgattgaaggtttcatggaggaagcaaaatggttcaatggtggaagtgcaccaaaattggaagagtatatagagaatggagtgtccacggcaggagcatacatggcttttgcacacatcttctttctcataggagaaggtgttacacaccaaaattcccaactcttcacccaaaaaccctaccccaaggtcttctccgccgccggccgcattcttcgcctctgggatgatctcggaaccgccaaggaagagcaagagcgaggagatctggcttcgtgcgtgcagttatttatgaaagagaagtcgttgacggaagaggaggcaagaagtcgcattttggaagagataaaaggattatggagggatctgaatggggaactggtctacaacaagaatttgccgttatccataatcaaagtcgcacttaacatggcgagagcttctcaagttgtgtacaagcacgatcaagacacttatttttcaagcgtagacaattatgtggatgccctcttcttcactcaataa

SEQ ID NO: 38 - DNA, 1725 bp, Ocimum basilicum
atgtcttgtgcacggatcaccgtaacattgccgtatcgctccgcaaaaacatcaattcaacggggaattacgcattgccccgcccttctccgcccacgcttctctgcttgcacacctttggcatccgcagtgcctctaagttcaactcctctcatcaacggggataactctccgcttaaaaacacacatcaacacgtggaggagaggagcagcaagaggagagaatatctgctggaggaaacggcgcgaaaactgcagagaaacgacaccgaatcggtggagaaactcaagctcatcgacaacatccaacggctgggaatcggctactattttgaggatgccatcgacgccgtactccgctcgcctttctccgccgaagaagaagaagacctcttcaccgctgctctgcgcttccgcttgctccgccacaatggcatccaagtcacccctgaaatattcctaaaattcaaggacgagagaggagaattcgacgaatcggacacgctagggttactgagcttgtacgaagcgtcaaatttgggggttacaggagaagagatactggaggaggctatggagttcgcggagcctcgcctgagacgatcactgtcagagctggcggcgccgcttcgtagtgaggtggcgcaagccctagatgtgccgaggcatctgagaatggcgaggttggaagccagacgattcatcgagcagtatggtaaacagagcgatcatgatggagaccttttggagctagcaattttggattataatcaagttcaggctcaacaccaatccgaactcactgaaattaccaggtggtggaagcaactgggtttggtggaaaagttgggtttcggtagagacagagcgttggagtgctttatgtggaccatggggatcctacctcaccctaaatactcttcttctagaatagaatcagccaaggcagctgctcttctgtacgtcatcgatgatattttcgatacctatggcaaaatggacgaactcatcctcttcaccgacgcaattcgaagatgggatttggaagcaatggagggtctacccgagtacatgaaaatatgctacatggcgttgtacaacaccaccaatgaaatatgctacagagtgctcaaggatactggacggatcgccctcccatacctcaaatctgtgtggatagagactattgaagcttatatggtggaggtgaagtggttcagtggtggaagtgccccaaagttggaagaatacattgagaatggggcatcaactgtaggggcatacatggttcttgtccacctattctttctcattggagaaggtctcacccaccaaaatgtcctatttttcaaacaaaaaccctatcacaagcctttctccgccgccggccggatttttcgcctttgggacgatcttggaacttctcaggaggaggaagaacgaggagatatggcgtcaagtatacggttatttatgaaagagtacaagttgtcgacggtagaggaggctagaagttgcgttttggaagagatatcccgtttatggaaggatcttaatgaagggctcatcagtataaaggacgccttgccattaaccatagtcaaagtcgcacttaacattgcacgaacttctcaagttgtatacaagcacgaacaacatacatatatgttgagtgttgataattacgtggaagccctcttcttcactcctcttctttcttcttag

SEQ ID NO: 39 - DNA, 1093 bp, Olea europaea
ctgatgtctttcttaaattcatggaaaagaacatgaaattcaaggawcatttggctcaagacaccataggcttattgagcttatacgaagcatcgtccatgggagcaaacggcgaagaaatattgtcagaggccaaggaatttaccgaaattcaccttagacagtcgatgcctcggttggtgccgcaacttcgycgacargttagttctgccttagagctcccgaggcacctcaggatggctcggttagaagctaggcgttacattgaagaatatggtaatgaaagtgaccatgatccagccgttttagaactggcaagattagattataacaaagtccagttacaacaccaaatggaattggctgaaataacaagatggtggaaacaattagggctagttgagaagctgagctttgcccgggacagacccttggaatgctttttatggacagtgggacttcttccagaacccaagtactctagctgcagaattgaactggctaagaccatagccattctattggtaattgatgatatttttgacacatatggcaaaatggaggaacttgttcttttcactgaagcaattcaaagatgggatcttgatgaattggaaactcttccaccgtacatgagaatatgttacatggctttgtacaacactaccaatgaaatctgctacaaaatcctcaaggagtatggattttgcgtccttccctaccttaaatccacgtggatagatatgattgaggggtttatggtagaggcaaattggttcaacggtggacatggaccaaatttggaggaatatatagagaatggtgtttcaacagcaggagcatatatggctttggtgcacctattctttcttataggggaaggagttacaaatgagaatattgctaaattgttgagaaaaccatatcckaaactcttttccgcwgcygggagaattcttcgtctstgggatgatctaggaactgccaaggaggaggaagaacgaggcgatcttgcatcrtgcatgcagatattaatgagagagaaraacatagattgtgaaaacgaaggcagaaattatattctgaaagctat

SEQ ID NO: 40 - DNA, 1752 bp, Olea europaea
atggactgtacaatgacgagtatttccttgttttcacagtcatctaatggtatttcaggaactgcaagaagtccatttcaatggccgataaatcatcgattttcgtcgggtcaacgtgatttcatttgcaagtcgttgcccgtgtcgtcgcctagtgcaacgccattgatccctgctgaaaatggtgctatgtataattatatacgtcaacctgtgatagttactcctgaagttgacgacggcacgaagcacagcgaattggtcgaaagaactcgacgagaactgcaaagaagtacaaaaccagttgagacgctaaaacttatagacaatcttcaacgattaggaattgcatattattttgaggatgatatcaatgcaatactggatcaattctctgatggcttgcctgatgaagatctcttcacaacagccttatgcttccgcttgctccgcgatcagcggctacaaactggttctgatgtctttcttaaattcatggaaaagaacatgaaattcaaggaacatttggctcaagacaccataggcttagtgagcttatacgaagcatcgtccatgggagcaaacggcgaagaaatattgtcagaggccaaggaatttaccgaaatgcaccttagacagtcgatgcctcagttggcgccgcaacttcgtcgacaagttagttctgccttagagctcccgaggcacctcaggatggctcggttagaagctaggcgttacattgaagaatatggtaatgaaagtgaccatgatccagcccttttggaactggcaagattagattataacaaagtccagttacagcaccaaatggaattggctgaaataacaagatggtggaaacaattagggctagttgagaagctgagctttgcccgggacagacccttggaatgctttttatggacggtgggacttcttccagaacccaagtactctagctgcagaattgaactggctaagaccatagccattctattggtaattgatgatatttttgacacatatggcaaaatggaggaacttgttcttttcactgaagcaattcaaagatgggatcttgatgaattggaaactcttccaccgtacatgagaatatgttacatggctttatacaacactaccaatgaaatctgctacaaaatcctcaaggagtatggattttgcgtccttccctaccttaaatccacgtggatagatatgattgaggggtttatggtagaggcaaattggttcaacggtggacatggaccaaatttggaggaatatatagaaaatggtgtttcaacagcaggagcatatatggctttggtgcacctattctttcttataggggaaggagttacaaatgagaatattgctaaattgttgagaaaaccatatccgaaactcttttccgctgctgggagaattcttcgtctgtgggatgatctaggaactgccaaggaggaggaagaacgaggcgatcttgcatcgtgcatgcagatattaatgagagagaagaacatagattgtgaaaacgaaggcagaaattatattctgaaagctataaacggcctatggaaagatctgaacgatgaactgatttcaccaaatgcaatgccattagccattaccaaagttgcattaaacatggctagagcttttgaagttgtttacaagcatgaagaggactcctacttttccagcgtcgataattatgtacaggctttgtttttcactcccattaattga

SEQ ID NO: 41 - DNA, 1755 bp, Phyla dulcis
atggcgagtgcaagaagcaccatatctttgtcctcacagtcatctcatcatgggttctccaaaaactcatttccatggcaactgaggcattcccgctttgttatgggttctcgagcacgtacctgcgcatgcatgtcatcatcagtatcactgcctactgcaacgacgtcgtcctcagtcattacaggcaacgatgccctcctcaaatacatacgtcagcctatggtaattcctttgaaagaaaaggagggcacgaagagacgagaatatctgctggagaaaactgcaagggaactgcagggaactacggaggcagcggagaaactgaaattcattgatacaatccaacggctgggaatctcttgctatttcgaggatgaaatcaacggcatactgcaggcggagttatccgatactgaccagcttgaggacggcctcttcacaacggctctacgcttccgtttgctccgtcactacggctaccaaatcgctcccgacgtcttcctaaaattcacggaccaaaatggaaaattcaaagaatccttagcggatgacacacaaggattagtcagcttatacgaagcatcatatatgggagcaaacggagaaaacatattagaagaagctatgaaattcaccaaaactcatctccaaggaagacaacatgcgatgagagaagtggctgaagccttggagcttccgaggcatctgagaatggccaggttagaagcaagaagatacatcgaacaatatggtacaatgattggacatgataaagacctcttggagctagtaatattggactataacaatgtccaggctcagcaccaagcggaactcgccgaaattgccagatggtggaaggagcttggtctagttgacaagttaactttcgcgcgagatagaccattggagtgctttttgtggactgtcggtcttctacctgaacccaaatactctgcttgccgaatcgagctcgcaaaaacaatagccattctattggtaatcgatgatatcttcgatacctatgggaaaatggaagaactcgctcttttcacggaggcaattagaagatgggatcttgaagctatggaaacccttcccgagtacatgaaaatatgctatatggcattgtacaataccaccaacgagatatgctacaaagtcctcaagaaaaatggatggagtgttctcccatacctaagatatacgtggatggacatgatagaaggttttatggtggaggcaaagtggttcaatggtggaagtgctccaaacttggaagagtacatagagaatggagtctcaacggctggggcatacatggctttggtgcatctcttctttctaattggggaaggtgtcagtgcgcaaaatgcccaaatattactgaagaaaccctatcctaagctcttctcggctgccggtcgaattcttcgcctttgggatgatcttggaacggctaaggaggaggaaggaagaggtgatcttgcatcgagcatacgtttattcatgaaagaaaagaacctaacaacggaagaggaagggagaaatggtatacaggaggagatatatagcttatggaaagacctaaacggagagctcatttctaaaggtaggatgccattggccatcatcaaagtggcacttaacatggctagagcttctcaagtggtgtacaagcatgacgaggactcttatttttcatgtgtagacaattatgtggaggccctgttcttcactcctctcctttga

SEQ ID NO: 42 - DNA, 1770 bp, Catharanthus roseus
atggcagccacaattagtaacctttctttcttagcaaaatctagggcactttcaaggccttcttcttcttcactttcatggctagaaaggcctaaaacttcatcgactatttgcatgtctatgccatcatcttcatcatcatcatcatcttcatccatgtctctgcctttggcaactccattgatcaaagacaatgaatctctcatcaagttcttgcgccaacccctggtgcttcctcatgaggttgatgacagcacaaaaaggagggaattgttggaaagaacaagaaaagaactagaattaaatgcagaaaaaccattggaggccttgaagatgatagatataattcaaagattgggattatcatatcattttgaagatgatattaattcaattctcacaggattttcaaatattagcagccaaactcatgaagatctcctcactgcttcactttgttttcgattgcttcgacacaatgggcataagatcaatcctgatatattccaaaaattcatggacaacaatggaaagtttaaagattcattaaaggatgacacattaggcatgttaagcttatatgaagcttcatatttgggagccaatggagaagaaatattgatggaagcccaagaattcaccaaaactcacctgaaaaactcattgccagccatggcaccatctctttctaagaaggtttctcaagctttagagcaaccaagacatagaagaatgttgaggttagaagctagaagatttattgaagaatatggtgctgaaaatgaccataatccagaccttcttgagcttgcaaaattggattataacaaagtccaatctctacaccaaatggaattgtctgagataacaaggtggtggaaacaattagggcttgtggataaactcacctttgctcgagatcgaccccttgaatgctttctttggacagtgggattattaccagagcctaagtattcaggttgcagaattgagcttgcaaaaaccatagccattttgcttgtcattgatgatatctttgatactcatggtaccctagatgagcttcttctattcactaatgccattaaaagatgggatcttgaggccatggaagatttaccagaatatatgagaatttgttacatggcattgtacaatactactaatgaaatttgctataaagttcttaaggaaaatggttggagtgttcttccttacctaaaggcaacgtggattgatatgattgaaggattcatggttgaagcagaatggttcaattctgattatgtaccaaacatggaagaatatgtagaaaatggagttagaacagcaggatcatatatggccttagtccatttgttctttctaatagggcaaggtgtcactgaagataatgtgaaattactgattaaaccctatccaaagctcttttcctcctcaggaagaatccttcgcctttgggatgatttgggaactgcaaaggaggaacaagaaagaggagacttggcatcaagcatacaattgtttatgagagagaaagagataaaatcagaagaagaaggaaggaaaggaatattggaaattatagagaatttatggaaagaattgaatggagaattagtttatagagaagaaatgcctcttgcaataatcaagacagcattcaacatggcaagagcttcccaagttgtgtatcaacatgaagaagacacctatttttcaagtgtagataattatgtaaaagctttgttttttacaccttgtttttaa

SEQ ID NO: 43 - DNA, 1761 bp, Picrorhiza kurrooa
atgggttgtattaggagcaccacacacacttcaactaattacgggattaattcaagactcggtttatctttcccggtccccattatatatcgccccgccggggcaagccgtaatatctccatttacatgtcttcatccgcggcgcctaacgttacacaggtggttgtcggagctaacgacgccctactccaatacacacatcaaccaatgttgataccgtcagaagtagatgcagacaccgagaggagggattatttgttggaaaaagctcgacaagaactgagccgaacaacaaaaccaacggataagcttaaactgattgacatgatccaacgacttggaattgggttttatttagaagacgatatcaacgctatactaaggactcaattctccgttaatgtgatagatgaagaagacctttccgccaccggattacgctttcgtatgctccgtcaaaatagcttcccaatctcttccgatattttcaagagattccaggatcaaaatgaaaaattcgatgaaactttaaccaacgacacgttgggggttattgagcttgtacgaagcatcatacatggggagccaaagggcgaagagatattggaggaagccatggaatttagcaagactcgcctcaagaggtccctttccagtctcccggcgcaaatcaaagccgacgtgggtcaagccttggagcttccaacacacttgaggatggccagattagaagctagacgattcatcgaacaatacgctacgcaaagtgatcacaatcctatccttttagaactagcaacactggactacaaccatgttcagtctcaacaccaaatggagctggttgaaatcactaggtggtggaaacagttgggtctagttgaccaattgagttttgctcgggatagacctttggaatgctttttgtggaccgttgggcttcttccagagcccaagtactctagttgtcgcatcgaactcgcaaaaacaatcgccgttttattggtaatcgatgatatttttgacacatatggcaaaatggatgatcttatcctcttcactgatgcaatccaaagatgggatctcggagcaatggaaagccttccggaatacatgaaaatatgttatatggcattgtacaacttaaccaacgagatatgttacaaagttctcaaagagaatggatggactgtcctgccctacttaaaatcgtcgtggatagatatgattgaaggctttatggtggaggcgaagtggtttaacgggggatgtgcaccaaatttggaagagtacgtagagaatggagtttcaactgccgggtcgtacatggcattggtgcatttgttctttttaataggggaaggtgttactgaacaaaatgcccaactcttaatgaagaaaccttatcctaaggccttttcttccgccggaagaattcttcgcctttgggatgatcttggaaccgccaaggaagaacaagaacgaggtgatctagcgtcgagcataaacttattcatgaaagagagaaatcttgcaacagaagaggaaggtagagaatgcatattggaagatatatatggatattggaaagatcttaatggagagctagtttctcagaaggagatgccattagccatcatcaaagtggcactgaacatggcaagggcttcccaagtagtgtacaagcatgaagaagattcttatttttccaatgtagacaattatgtacaagcgcttttatttactcctcttattatttga

SEQ ID NO: 44 - DNA, 1785 bp, Valeriana officinalis
atgataacttcatcgtcgtccgttcgatctttatgctgtccgaaaaccagtatcatttcaggcaaacttcttccttcattgctcctcactaacgtaataaatgtcagtaatggtacgagtagtcgcgcttgcgtttccatgtcgtctttaccggtttccaagtccactgcctcctccattgcagctccgttggtccgggacaacggctccgcccttaacttttttcctcaagctccacaggttgaaatagatgaaagcagtagaataatggagttggtagaagccactagaagaacactcagaaacgaatcttctgattctacggagaagatgaggttgatcgactcccttcaacgcctgggattgaaccaccatttcgaacaagacataaaggagatgctacaagatttcgcaaatgagcacaagaatacaaaccaagatctctttactacttcactacgttttcgattgctaagacataatggctttaatgtcacgcctgatgtattcaataaattcacggaggaaaatggcaagtttaaggaatcattaggcgaggacacaattgggattttaagtttgtatgaagcgtcgtatttagggggaaagggggaagaaatattatcggaggcaatgaaatttagcgaatctaaattaagagaatcttctggacacgtggcacstcatattcgtcgacagattttccagtcgttggagcttccgaggcatttgagaatggctaggttagaatcccgaagatacatcgaagaagattacagcaatgaaattggggctgattcttccttgcttgaattagcaaaactcgatttcaattcagtacaagcacttcaccaaatggaattaaccgaaatttctaggtggtggaaacaattgggtttgtctgataagctaccatttgcacgcgatcgaccgttggaatgcttcttgtggaccgtcggattacttcctgaaccaaaatattctggttgtaggattgagttggccaaaaccattgccgttttgctcgttattgatgacatattcgacacttatggctcgtatgatcaacttattctcttcacaaacgcaatcagaagatgggatctagatgcaatggatgaacttcccgagtacatgaaaatatgctacatggcattgtacaacacgactaacgaaatatgttacaaagtacttaaagaaaacggatggagtgttcttccttacctcgaacgaacgtggatagacatggttgaaggattcatgctagaagccaagtggttaaacagcggcgaacaaccgaatttggaagcatacatagaaaacggcgttaccacagccggctcttacatggctttagtacacttgtttttcctcataggagacggcgttaacgatgagaacgtgaaattactattagacccttaccctaaactgttctcatcggctgggcgcattcttcgactgtgggacgatttgggtactgctaaggaggaacaagagagaggggatgtatcttcaagcatacaattgtacatgaaggagaagaatgtgagatcagagagtgaaggaagggagggaatagtagagataatatacaacttgtggaaagacatgaatggagaattgattggttcaaacgcattgccgcaagcaatcatagagacctcattcaacatggcaagaacatcacaagttgtgtatcaacacgaagatgatacttatttctctagtgtcgataactatgtacaatctctcttcttcactcctgtttctgtttctgtttaa

SEQ ID NO: 45 - DNA, 1778 bp, Vitis vinifera
atgagcaggttcgttaccatgccttcacatgtgctgccatcttcctttgtcgctccttcacttcaagtctcatcgtctccttgttcatggcggacgcgcccctccccttgcaccagctgccatttatcgccatcatcatcgtcaaagccactgcttggaagccatgattattctttgctcaagtccttaactctatctccacatgcggtgaattctgaagctgatagcagcaccagaaggatgaaagaagtgaaggaaagaacctgggaggcattctatagagcttgggattcaagggctgctatggagatggtagacacagttgagaggctaggtctatcataccattttgaagacgagataaatgcactattgcaaaggttctgcgactggaatgccagtgaagatctcttcaccactgcccttcgtttccggttgcttcgccaaaatggattcccaactcattctgatgtttttggcaagttcatggataagaatggaaaattcaaggaaagcttgactgaagacatatggggtatgctgagtttacatgaggcatctcacttgggggccaaaaatgaagaagtattagcagaagccaaggagttcacaaggattcacctcatacagtctatgccgcacatggaaccccattttagcagccatgtcggccgggcactcgagcttcccaggcacttgaggatggtgaggttggaggctagaaactatataggcgaatatagcagggaaagcaacccgaacttagcttttctagagctagcaaaattggatttcgacatggttcaatcactgcaccagaaagagttggctgagatagtaaggtggtggaaacagttgggtcttgttgataagcttgattttgctcgagaccggccgatggagtgcttcttatggacagtggggatatttccagatcccaggcattcaagctgccgcatcgaattaaccaaggccattgctatcttactagttattgatgatatttacgactcttatggatctttggacgagcttgctctcttcactgatgcagtaaaaagatgggatcttggtgcaatggatcagctccctgagtacatgaagatatgttacatggcattgtacaacactactaatgacattgcatacaggatcttgaaggagcatggatggagcgtcatagaagacctaaaacgaacgtggatggacatctttggagctttcctagctgaagcctattgtttcaaaggtggccacgtaccctcactagaagagtatctaactaatgcagttaccactggaggcacatacatggccttggtgcatgctttttttcttatggggcaaggtgttactagggaaaatatggccatgttgaagccctatcccaacatcttctcctgctcagggaaaattcttcggctttgggatgacttgggaactgcaagagaggagcaagagagaggagataacgcgtcgagtatagaatgctacaagcgagagcgagagatggatactgtgttggaagatgaagcctgcaggaaacacataaggcagatgatccagagcttgtgggtagagctcaatggggagctggttgcttcgtctgcattgcctctttcaatcatcaaagctgcctttaacctctcaagaactgcacaagtcatttaccagcatggagatgataacaagacttctagtgttgaagatcatgtacaagcattgctctttaggcctgtttcctctaatgggcatgctcaaattaccatgcattagat

SEQ ID NO: 46 - DNA, 1788 bp, Vitis vinifera
atggctttcaatatgagcaggttcgttaccatgccttcacatgtgctgccatcttcctttgtcgctccttcacttcaagtctcatcgtctccttgttcatggcggacgcgcccctccccttgcaccagctgccatttatcgccatcatcatcgtcaaagccactgcttggaagccatgattattctttgctcaagtccttaactctatctccacatgcggtgaattctgaagctgatagcagcaccagaaggatgaaagaagtgaaggaaagaacctgggaggcattctatagagcttgggattcaagggctgctatggagatggtagacacagttgagaggctaggtctatcataccattttgaagacgagataaatgcactattgcaaaggttctgcgactggaatgccagtgaagatctcttcaccactgcccttcgtttccggttgcttcgccaaaatggattcccaactcattctgatgtttttggcaagttcatggataagaatggaaaattcaaggaaagcttgactgaagacatacggggtatgctgagtttacatgaggcatctcacttgggggccaaaaatgaagaagtattagcagaagccaaggagttcacaaggattcacctcatacagtctatgccgcacatggaaccccattttagcagccatgtcggccgggcactcgagcttcccaggcacttgaggatggtgaggttggaggctagaaactatataggcgaatatagcagggaaagcaacccgaacttagcttttctagagctagcaaaattggatttcgacatggttcaatcactgcaccagaaagagttggctgagatattaaggtggtggaaacagttgggtcttgttgataagcttgattttgctcgagaccggccgatggagtgcttcttatggacagtggggatatttccagatcccagacattcaagctgccgcatcgaattaaccaaggccattgctatcttactagttattgatgatatttacgactcttatggatctttggacgagcttgctctcttcactgatgcagtaaaaagatgggatcttggtgcaatggatcagctccctgagtacatgaagatatgttacatggcattgtacaacactactaatgacattgcatacaggatcttgaaggagcatggatggagcgtcatagaacacctaaaacgaacgtggatggacatctttggagctttcctagctgaagcctattgtttcaaaggtggccacgtaccctcactagaagagtatctaactaatgcagttaccactggaggcacatacatggccttggtgcatgctttttttcttatggggcaaggtgttactagggaaaatatggccatgttgaagccctatcccaacatcttctcctgctcagggaaaattcttcggctttgggatgacttgggaactgcaagagaggagcaagagagaggagataacgcgtcgagtatagaatgctacaagcgagagcgagagatggatactgtgttggaagatgaagcctgcaggaaacacataaggcagatgatccagagcttgtgggtagagctcaatggggagctggttgcttcgtctgcattgcctctttcaatcatcaaagctgcctttaacctctcaagaactgcacaagtcatttaccagcatggagatgataacaagacttctagtgttgaagatcatgtacaagcattgctctttaggcctgtttcctctaatgggcatgctcaaattaccatgcattag

SEQ ID NO: 47 - DNA, 1377 bp, Artificial sequence
  Single strand DNA oligonucleotide
atggcaagctctatgctttcctccgctgccgttgtcaccagtccagctcaggctactatggttgcccctttcactggtctcaaatcctctgcctcttttcccgtcacacgtaaggccaataacgatatcacttctatcacctctaacggtggccgagtttcttgcatgaaagtgtggccttttgtcaaagactttaaaccccaagctcttggcgatactaaccttttcaagcccattaagattggaaacaatgagttgcttcacagggctgttatccctcctcttactagaatgagagcacaacacccaggaaacattcctaatcgcgattgggccgttgaatactacgctcaacgagcacagaggccaggcactttgatcattactgagggaacttttccttcaccacaatctggcggatatgacaatgcaccaggaatctggagcgaggaacagattaaggagtggaccaagatcttcaaggctattcatgagaataagtcatttgcatgggttcagctctgggttctcgggtgggcagcatttcctgacaccttggctagggacgggttgaggtacgattccgctagtgataacgtgtacatgaatgctgagcaggaggagaaagcaaaaaaggctaacaatccccagcattccatcaccaaggatgagattaagcaatacgtcaaggaatatgtgcaagctgccaaaaactcaatagccgccggggcagacggtgttgagattcattccgctaatggctacttgcttaaccagtttcttgatcctcactcaaacaaccgcacagacgaatatgggggatctatagagaatagagcacgtttcaccttggaagtcgttgacgccgttgttgacgctattggtccagagaaagtgggtctgcgactgtcaccatatggagttttcaattcaatgagcggtggagccgaaaccggcatagtcgctcagtacgcatatgtgttgggagaattggaaaggagagcaaaagctggtaaacgtctggcctttgttcaccttgtggagccacgtgtgacaaatcctttcctgactgaaggagaaggcgaatataacggcggatctaacaagttcgcttactccatctggaaaggaccaatcattagggccggaaacttcgcccttcatccagaggtcgttagagaagaggtgaaagatccaaggacactcattggttatggccgctttttcatctcaaatccagatctcgttgatagattggagaaggggttgcccttgaacaagtatgatagggacactttctataagatgtccgctgagggttacattgattaccccacttatgaagaggctttgaagctcggatgggataagaactaa

SEQ ID NO: 48 - DNA, 1767 bp, Saccharomyces cerevisiae
tcgaggtaacctgccttgttcctcctggtgactttctaaaatataaaaggaaaagcatatctctagtttcgagttttttcttcatactttatttccttatgttaaacggtccagatatagaataaatcatcatattaagctaaatatagacgataatatagtatcgataatgccatttgttaaggactttaagccacaagctttgggtgacaccaacttattcaaaccaatcaaaattggtaacaatgaacttctacaccgtgctgtcattcctccattgactagaatgagagcccaacatccaggtaatattccaaacagagactgggccgttgaatactacgctcaacgtgctcaaagaccaggaaccttgattatcactgaaggtacctttccctctccacaatctgggggttacgacaatgctccaggtatctggtccgaagaacaaattaaagaatggaccaagattttcaaggctattcatgagaataaatcgttcgcatgggtccaattatgggttctaggttgggctgctttcccagacacccttgctagggatggtttgcgttacgactccgcttctgacaacgtgtatatgaatgcagaacaagaagaaaaggctaagaaggctaacaacccacaacacagtataacaaaggatgaaattaagcaatacgtcaaagaatacgtccaagctgccaaaaactccattgctgctggtgccgatggtgttgaaatccacagcgctaacggttacttgttgaaccagttcttggacccacactccaataacagaaccgatgagtatggtggatccatcgaaaacagagcccgtttcaccttggaagtggttgatgcagttgtcgatgctattggccctgaaaaagtcggtttgagattgtctccatatggtgtcttcaacagtatgtctggtggtgctgaaaccggtattgttgctcaatatgcttatgtcttaggtgaactagaaagaagagctaaagctggcaagcgtttggctttcgtccatctagttgaacctcgtgtcaccaacccatttttaactgaaggtgaaggtgaatacaatggaggtagcaacaaatttgcttattctatctggaagggcccaattattagagctggtaactttgctctgcacccagaagttgtcagagaagaggtgaaggatcctagaacattgatcggttacggtagattttttatctctaatccagatttggttgatcgtttggaaaaagggttaccattaaacaaatatgacagagacactttctacaaaatgtcagctgagggatacattgactaccctacgtacgaagaagctctaaaactcggttgggacaaaaattaatagtgttaaccgtactttgtagcaccatttctttttcttctaatttatataattaatgaatatatatatttgggataaatagcctttatgaaatattacagactttttgcttgggcagcaacagatttatatctttgtggaaaggagtaaactgggctttcactttagatcattattcacataaacttcttttagaatatgtttgaatgtaaatccacaaaatggaatcggtcattctacatcatgaatattaatttaccttctttccttttatatgctgtcattcatcatcctattacattatcaatcgttgcatttcagcttccattaatttcgacgactgtgtcccaatcctatgtcatcttcttacaccgcgtgtaataatatagtagtaa

SEQ ID NO: 49 - DNA, 1203 bp, Saccharomyces cerevisiae
atgccatttgttaaggactttaagccacaagctttgggtgacaccaacttattcaaaccaatcaaaattggtaacaatgaacttctacaccgtgctgtcattcctccattgactagaatgagagcccaacatccaggtaatattccaaacagagactgggccgttgaatactacgctcaacgtgctcaaagaccaggaaccttgattatcactgaaggtacctttccctctccacaatctgggggttacgacaatgctccaggtatctggtccgaagaacaaattaaagaatggaccaagattttcaaggctattcatgagaataaatcgttcgcatgggtccaattatgggttctaggttgggctgctttcccagacacccttgctagggatggtttgcgttacgactccgcttctgacaacgtgtatatgaatgcagaacaagaagaaaaggctaagaaggctaacaacccacaacacagtataacaaaggatgaaattaagcaatacgtcaaagaatacgtccaagctgccaaaaactccattgctgctggtgccgatggtgttgaaatccacagcgctaacggttacttgttgaaccagttcttggacccacactccaataacagaaccgatgagtatggtggatccatcgaaaacagagcccgtttcaccttggaagtggttgatgcagttgtcgatgctattggccctgaaaaagtcggtttgagattgtctccatatggtgtcttcaacagtatgtctggtggtgctgaaaccggtattgttgctcaatatgcttatgtcttaggtgaactagaaagaagagctaaagctggcaagcgtttggctttcgtccatctagttgaacctcgtgtcaccaacccatttttaactgaaggtgaaggtgaatacaatggaggtagcaacaaatttgcttattctatctggaagggcccaattattagagctggtaactttgctctgcacccagaagttgtcagagaagaggtgaaggatcctagaacattgatcggttacggtagattttttatctctaatccagatttggttgatcgtttggaaaaagggttaccattaaacaaatatgacagagacactttctacaaaatgtcagctgagggatacattgactaccctacgtacgaagaagctctaaaactcggttgggacaaaaattaa

SEQ ID NO: 50 - DNA, 1203 bp, Saccharomyces cerevisiae
atgccatttgttaaggactttaagccacaagctttgggtgacaccaacttattcaaaccaatcaaaattggtaacaatgaacttctacaccgtgctgtcattcctccattgactagaatgagagcccaacatccaggtaatattccaaacagagactgggccgttgaatactacgctcaacgtgctcaaagaccaggaaccttgattatcactgaaggtacctttccctctccacaatctgggggttacgacaatgctccaggtatccggtccgaagaacaaattaaagaatggaccaagattttcaaggctattcatgagaataaatcgttcgcatgggtccaattatgggttctaggttgggctgctttcccagacacccttgctagggatggtttgcgttacgactccgcttctgacaacgtgtatatgaatgcagaacaagaagaaaaggctaagaaggctaacaacccacaacacagtataacaaaggatgaaattaagcaatacgtcaaagaatacgtccaagctgccaaaaactccattgctgctggtgccgatggtgttgaaatccacagcgctaacggttacttgttgaaccagttcttggacccacactccaataacagaaccgatgagtatggtggatccatcgaaaacagagcccgtttcaccttggaagtggttgatgcagttgtcgatgctattggccctgaaaaagtcggtttgagattgtctccatatggtgtcttcaacagtatgtctggtggtgctgaaaccggtattgttgctcaatatgcttatgtcttaggtgaactagaaagaagagctaaagctggcaagcgtttggctttcgtccatctagttgaacctcgtgtcaccaacccatttttaactgaaggtgaaggtgaatacaatggaggtagcaacaaatttgcttattctatctggaagggcccaattattagagctggtaactttgctctgcacccagaagttgtcagagaagaggtgaaggatcctagaacattgatcggttacggtagattttttatctctaatccagatttggttgatcgtttggaaaaagggttaccattaaacaaatatgacagagacactttctacaaaatgtcagctgagggatacattgactaccctacgtacgaagaagctctaaaactcggttgggacaaaaattaa

SEQ ID NO: 51 - DNA, 1203 bp, Saccharomyces cerevisiae
atgtcatttgtaaaggatttcaagccacaagctttgggtgacaccaacttattcaaaccaatcaaaattggtaacaatgaacttctacaccgtgctgtcattcctccattgactagaatgagagcccaacatccaggtaatattccaaacagagactgggccgttgaatactacgctcaacgtgctcaaagaccaggaaccttgattatcactgaaggtacctttccctctccacaatctgggggttacgacaatgctccaggtatctggtccgaagaacaaattaaagaatggaccaagattttcaaggctattcatgagaagaaatcgttcgcatgggtccaattatgggttctaggttgggctgctttcccagacacccttgctagggatggtttgcgttacgactccgcttctgacaacgtgtatatgaatgcagaacaagaagaaaaggctaagaaggctaacaacccacaacacagtataacaaaggatgaaattaagcaatacgtcaaagaatacgtccaagctgccaaaaactccattgctgctggtgccgatggtgttgaaatccacagcgctaacggttacttgttgaaccagttcttggacccacactccaataacagaaccgatgagtatggtggatccatcgaaaacagagcccgtttcaccttggaagtggttgatgcagttgtcgatgctattggccctgaaaaagtcggtttgagattgtctccatatggtgtcttcaacagtatgtctggtggtgctgaaaccggtattgttgctcaatatgcttatgtcttaggtgaactagaaagaagagctaaagctggcaagcgtttggctttcgtccatctagttgaacctcgtgtcaccaacccatttttaactgaaggtgaaggtgaatacaatggaggtagcaacgaatttgcttattctatctggaagggcccaattattagagctggtaactttgctctgcacccagaagttgtcagagaagaggtgaaggatcctagaacattgatcggttacggtagattttttatctctaatccagatttggttgatcgtttggaaaaagggttaccattaaacaaatatgacagagacactttctacaaaatgtcagctgagggatacattgactaccctacgtacgaagaagctctaaaactcggctgggacaagcattaa

SEQ ID NO: 52 - DNA, 1203 bp, Candida sake
atgtcatttgttaaggatttcaagccacaagctttgggtgacaccaacttattcaaaccaatcaaaattggtaacaatgaacttctacaccgtgctgtcattcctccattgactagaatgagagcccaacatccaggtaatattccaaacagagactgggccgttgaatactacgctcaacgtgctcaaagaccaggaaccttgattatcactgaaggtacctttccctctccacaatctgggggttacgacaatgctccaggtatctggtccgaagaacaaattaaagaatggaccaagattttcaaggctattcatgagaataaatcgttcgcatgggtccaattatgggttctaggttgggctgctttcccagacacccttgctagggatggtttgcgttacgactccgcttctgacaacgtgtatatgaatgcagaacaagaagaaaaggctaagaaggctaacaacccacaacacagtataacaaaggatgaaattaagcaatacgtcaaagaatacgtccaagctgccaaaaactccattgctgctggtgccgatggtgttgaaatccacagcgctaacggttacttgttgaaccagttcttggacccacactccaataacagaaccgatgagtatggtggatccatcgaaaacagagcccgtttcaccttggaagtggttgatgcagttgtcgatgctattggccctgaaaaagtcggtttgagattgtctccatatggtgtcttcaacagtatgtctggtggtgctgaaaccggtattgttgctcaatatgcttatgtcttaggtgaactagaaagaagagctaaagctggcaagcgtttggctttcgtccatctagttgaacctcgtgtcaccaacccatttttaactgaaggtgaaggtgaatacaatggaggtagcaacaaatttgcttattctatctggaagggcccaattattagagctggtaactttgctctgcacccagaagttgtcagagaagaggtgaaggatcctagaacattgatcggttacggtagattttttatctctaatccagatttggttgatcgtttggaaaaagggttaccattaaacaaatatgacagagacactttctacaaaatgtcagctgagggatacattgactaccctacgtacgaagaagctctaaaactcggctgggacaagaagtaa

SEQ ID NO: 53 - DNA, 1203 bp, Kazachstania exigua
atgccatttgtcaaggattttaagccacaagctttgggtgacaccaacttattcaaaccaatcaaaattggtaacaatgaacttctacaccgtgctgtcattcctccattgactagaatgagagcccaacatccaggtaatattccaaacagagactgggccgttgaatactacgctcaacgtgctcaaagaccaggaaccttgattatcactgaaggtacctttccctctccacaatctgggggttacgacaatgctccaggtatctggtccgaagaacaaattaaagaatggaccaagattttcaaggctattcatgagaagaaatcgttcgcatgggtccaattatgggttctaggttgggctgctttcccagacacccttgctagggatggtttgcgttacgactccgcttctgacaacgtgtatatgaatgcagaacaagaagaaaaggctaagaaggctaacaacccacaacacagtataacaaaggatgaaattaagcaatacgtcaaagaatacgtccaagctgccaaaaactccattgctgctggtgccgatggtgttgaaatccacagcgctaacggttacttgttgaaccagttcttggacccacactccaataacagaaccgatgagtatggtggatccatcgaaaacagagcccgtttcaccttagaagtggttgatgcagttgtcgatgctattggccctgaaaaagtcggtttgagattgtctccatatggtgtcttcaacagtatgtctggtggtgctgaaaccggtattgttgcccaatatgcttatgtcttaggtgaactagaaagaagagctaaagctggcaagcgtttggctttcgtccatctagttgaacctcgtgtcaccaacccatttttaactgaaggtgaaggtgaatacaatggaggtagcaacgaatttgcttattctatctggaagggcccaattattagagctggtaactttgctctgcacccagaagttgtcagagaagaggtgaaggatcctagaacattgatcggttacggtagattttttatctctaatccagatttggttgatcgtttggaaaaagggttaccattaaacaaatatgacagagacactttctacaaaatgtcagctgagggatacattgactaccctacgtacgaagaagctctaaaactcggctgggacaagcattaa

SEQ ID NO: 54 - DNA, 1203 bp, Naumovozyma castellii
atgtcatttgtaaaagattttaagccacaagctttgggtgacaccaacttattcaaaccaaccaaaattggtaacaatgaacttctacaccgtgctgtcattcctccattgactagaatgagagcccaacatccaggtaatattccaaacagagactgggccgttgaatactacgctcaacgtgctcaaagaccaggaaccttgattatcactgaaggtacctttccctctccacaatctgggggttacgacaatgctccaggtatctggtccgaagaacaaattaaagaatggaccaagattttcaaggctattcatgagaataaatcgttcgcatgggtccaattatgggttctaggttgggctgctttcccagacacccttgctagggatggtttgcgttacgactccgcttctgacaacgtgtatatgaatgcagaacaagaagaaaaggctaagaaggctaacaacccacaacacagtataacaaaggatgaaattaagcaatacgtcaaagaatacgtccaagctgccaaaaactccattgctgctggtgccgatggtgttgaaatccacagcgctaacggttacttgttgaaccagttcttggacccacactccaataacagaaccgatgagtatggtggatccatcgaaaacagagcccgtttcaccttggaagtggttgatgcagttgtcgatgctattggccctgaaaaagtcggtttgagattgtctccatatggtgtcttcaacagtatgtctggtggtgctgaaaccggtattgttgctcaatatgcttatgtcttaggtgaactagaaagaagagctaaagctggcaagcgtttggctttcgtccatctagttgaacctcgtgtcaccaacccatttttaactgaaggtgaaggtgaatacaatggaggtagcaacaaatttgcttattctatctggaagggcccaattattagggctggtaactttgctctgcacccagaagttgtcagagaagaggtgaaggatcctagaacattgatcggttacggtagattttttatctctaatccagatttggttgatcgtttggaaaaagggttaccattaaacaaatatgacagagacactttctacaaaatgtcagctgagggatacattgactaccctacgtacgaagaagctctaaaactcggctgggacaaacagtaa

SEQ ID NO: 55 - DNA, 1203 bp, Kazachstania spencerorum
atgtcatttgtaaaggactttaagccacaagctttgggtgacaccaacttattcaaaccaatcaaaattggtaacaatgaacttctacaccgtgctgtcattcctccattgactagaatgagagcccaacatccaggtaatattccaaacagagactgggccgttgaatactacgctcaacgtgctcaaagaccaggaaccttgattatcactgaaggtacctttccctctccacaatctgggggttacgacaatgctccaggtatctggtccgaagaacaaattaaagaatggaccaagattttcaaggctattcatgagaagaaatcgttcgcatgggtccaattatgggttctaggttgggctgctttcccagacacctttgctagggatggtttgcgttacgactccgcttctgacaacgtgtatatgaatgcagaacaagaagaaaaggctaagaaggctaacaacccacaacacagtataacaaaggatgaaattaagcaatacgctaaagaatacgtccaagctgccaaaaactccattgctgctggtgccgatggtgttgaaatccacagcgctaacggttacttgttgaaccagttcttggacccacactccaataacagaaccgatgagtatggtggatccatcgaaaacagagcccgtttcaccttagaagtggttgatgcagttgtcgatgctattggccctgaaaaagtcggtttgagattgtctccatatggtgtcttcaacagtatgtctggtggtgctgaaaccggtattgttgctcaatatgcttatgtcttaggtgaactagaaagaagagctaaagctggcaagcgtttggctttcgtccatctagttgaacctcgtgttaccaacccatttttaactgaaggtgaaggtgaatacaatggaggtagcaacgaatttgcttattctatctggaagggcccaattattagagctggtaactttgctctgcacccagaagttgtcagagaagaggtgaaggatcctagaacattgatcggttacggtagattttttatctctaatccagatttggttgatcgtttggaaaaagggttaccattaaacaaatatgacagagacactttctacaaaatgtcagctgagggatacattgactaccctacgtacgaagaagctctaaaactcggttgggacaaacagtaa

SEQ ID NO: 56 - DNA, 1203 bp, Kazachstania spencerorum
atgtcatttgttaaagatttcaagccacaagctttgggtgacaccaacttattcaaaccaatcaaaattggtaacaatgaacttctacaccgtgctgtcattcctccattgactagaatgagagcccaacatccaggtaatattccaaacagagactgggccgttgaatactacgctcaacgtgctcaaagaccaggaaccttgattatcactgaaggtacctttccctctccacaatctgggggttacgacaatgctccaggtatctggtccgaagaacaaattaaagaatggaccaagattttcaaggctattcatgagaagaaatcgttcgcatggatccaattatgggttctaggttgggctgctttcccagacacctttgctagggatggtttgcgttacgactccgcttctgacaacgtgtatatgaatgcagaacaagaagaaaaggctaagaaggctaacaacccacaacacagtataacaaaggatgaaattaagcaatacgtcaaagaatacgtccaagctgccaaaaactccattgctgctggtgccgatggtgttgaaatccacagcgctaacggttacttgttgaaccagttcttggacccacactccaataacggaaccgatgagtatggtggatccatcgaaaacagagcccgtttcaccttagaagtggttgatgcagttgtcgatgctattggccctgaaaaagtcggtttgagattgtctccatatggtgtcttcaacagtatgtctggtggtgctgaaaccggtattgttgctcaatatgcttatgtcttaggtgaactagaaagaagagctaaagctggcaagcgtttggctttcgtccatctagttgaacctcgtgtcaccaacccatttttaactgaaggtgaaggtgaatacaatggaggtagcaacgaatttgcttattctatctggaagggcccaattattagagctggtaactttgctctgcacccagaagttgtcagagaagaggtgaaggatcctagaacattgatcggttacggtagattttttatctctaatccagatttggttgatcgtttggaaaaagggttaccattaaacaaatatgacagagacactttctacaaaatgtcagctgagggatacattgactaccctacgtacgaagaagctctaaaactcggttgggacaagcagtaa

SEQ ID NO: 57 - DNA, 1203 bp, Nakaseomyces bacillisporus
atgtcatttgttaaggattttaagccacaagctttgggtgacaccaacttattcaaaccaatcaaaattggtaacaatgaacttctacaccgtgctgtcattcctccattgactagaatgagagcccaacatccaggtaatattccaaacagagactgggccgttgaatactacgctcaacgtgctcaaagaccaggaaccttgattatcactgaaggtacctttccctctccacaatctgggggttacgacaatgctccaggtatctggtccgaagaacaaattaaagaatggaccaagattttcaaggctattcatgagaagaaatcgttcgcatgggtccaattatgggttctaggttgggctgctttcccagacacctttgctagggatggtttgcgttacgactccgcttctgacaacgtgtatatgaatgcagaacaagaagaaaaggctaagaaggctaacaacccacaacacagtataacaaaggatgaaattaagcaatacgtcaaagaatacgtccaagccgccaaaaactccattgctgctggtgccgatggtgttgaaatccacagcgctaacggttacttgttgaaccagttcttggacccacactccaataacagaaccgatgagtatggtggatccatcgaaaacagagcccgtttcaccttagaagtggttgatgcagttgtcggtgctattggccctgaaaaagtcggtttgagattgtctccatatggtgtcttcaacagtatgtctggtggtgctgaaaccggtattgttgctcaatatgcttatgtcttaggtgaactagaaagaagagctaaagttggcaagcgtttagctttcgtccatctagttgaacctcgtgtcaccaacccatttttaactgaaggtgaaggtgaatacaatggaggtagcaacgaatttgcttattctatctggaagggcccaattattagagctggtaactttgctctgcacccagaagttgtcagagaagaggtgaaggatcctagaacattgatcggttacggtagattttttatctctaatccagatttggttgatcgtttggaaaaagggttaccattaaacaaatatgacagagacactttctacaaaatgtcagctgagggatacattgactaccctacgtacgaagaagctctaaaactcggttgggacaagcagtaa

SEQ ID NO: 58 - DNA, 1083 bp, Artificial sequence
  Single strand DNA oligonucleotide
atggctaagtctccagaaaccgagcatcctgttaaagccttgggatgggctgccactgataattccggaacttttagtccctttaacttctcaagaagagccaccggagagagggatgttcaatttagagttctttactgcggagtgtgtcattcagatcttcacatggttaagaacgaatggggtgtcacacattatcccatagttcctgggcacgaaatcgttggaattgtgaccgaagtgggtagtaaagtggagaaggttaagatcggagataaggttggtgtgggcgttctcgtgggatcatgtcgccagtgtgatcaatgtagtaacgacttggagaattactgttacaagcagattctcacatatggcgctccatatctggacgggacaatagctagaggaggctattctgatattatggtcgcagatgagcacttcataattaggtggcctgaaaacttcccacttgatgcaggcgctccacttctctgtgcaggcattacaacttattctccattgaaatacttcggtttggacaagcctgggcttcgcgttggcgttaacggactcgggggtctcggacacgttgctgtgaagtttgctaaggcattcggaacaaaagtcactgtgatctcaactagcctgagcaagaaagaagaggctatgcagcatcttggagtcgatgaatttgtggtcagtactgatccccaacaaatgcaagccgcagtggggactcttgacggaatcatagataccgtgtccgcacctcatcctatagtgcctctgctttcccttttgaagccacatggtaagctcatagttgtggggcttccagataagcccttgcaacttcctgtgtttcctctcatccaggggagaagaacaattgccggtagcgggataggtggactcaaggaaacacaagaaatgatagacttcgcagccaagaacaatatcgttgccgacgtcgaagtgatcccaattgattatattaacacagctatggatcgccttctgaaaagcgacgttaagtaccgctttgtgatcgatgttgagaagagtttgaaaccccagtaa

SEQ ID NO: 59 - DNA, 1083 bp, Ocimum basilicum
atggcgaaatcaccagaaacagagcacccagtgaaggccttgggatgggcagccacagacaattctgggaccttttctcccttcaacttctctaggagggccaccggcgagcgtgacgtgcagttcagggtattgtattgtggcgtttgtcattcagatctccacatggtcaagaatgagtggggcgttactcactatcccattgttcctgggcatgaaatagtgggtattgtgactgaggtgggcagcaaggtggagaaggtgaagatcggagacaaagttggtgtaggggtgttggtgggatcatgccggcagtgtgatcagtgcagcaatgatcttgaaaactactgctacaagcaaatcctgacctacggtgccccttacctcgatggaaccattgctcgcggaggctactctgatatcatggtggcagacgagcatttcatcatccgctggcctgaaaacttccccctcgacgctggagcccctctcctctgtgctggtatcactacttacagtcctctcaagtattttggactggacaagcctggactcagggtgggcgtcaacggccttggtggcctgggtcatgtagctgtcaaatttgctaaagctttcgggactaaggtgactgtcatcagcacatctctttccaagaaggaggaagctatgcaacatcttggcgtggacgagtttgtagtcagcaccgatccacagcagatgcaggctgcagtaggcacactagatggtatcattgacacggtctctgcacctcatcccattgttccgttgctgagcttgctgaagccacacgggaagcttatcgtggttggccttcccgataagcccctgcagcttccggttttccccttgattcaaggtaggaggactatagccgggagtggcatcggcgggcttaaggagacgcaggagatgatcgatttcgcggccaagaacaacatagtagcggacgtggaggtgatccccatcgactatataaacacagcaatggatcggcttctcaaatctgatgtcaagtaccgtttcgtcatcgatgttgaaaaatccttgaaacctcagtga

SEQ ID NO: 60 - DNA, 1646 bp, Ocimum americanum
acaattccgggaccttttctccattcaagttttctaggaggtacggaaatcaaaatattattgtgtgtgtgtgtgtgcgcgcgagagagagattaattcttgtttgatggtggatgtgtaccggacgcgggcatggcgaaatcaccagaaacagagcacccagtgaaggccttgggatgggcagccacagacaattctgggaccttttctcccttcaacttctctaggagggctaccggtgagcgtgatgtgcagttcagggtactgtattgtggcgtttgtcattcagatctgcacatggtcaagaatgagtggggggttactctctatcccattgttccagggtacgtaccactctttcatctcacttttttgttatgtgcttgcctgtatgacttcctttttaggcattatactagtaatcatagaattcaacaatatatcaatttttttatttggtctgatggcttcaacttttcattgtaaaatttaatattgtttctcctagcttcacctccggatgaatttgagttttaggggaagctttatcatctagaaaaatattgagcttagaaaatgaaaggatgaagcaaaatgcagtgttattggtggtgcttatactagtttgttgcgcagacatgaaattgtgggtattgtgactgaggtgggcagcaaggtggaaaaggtaaaagtcggagacaaagttggtgttggagtgttggtgggatcatgccggcagtgtgatcagtgcagcaatgatcttgaaaactactgctacaagcaaatactcacctacggcgccccttaccttgatggaaccattactcgcggaggctactccgatacagacgagcatttcatcatccgttggcctgaaaacttccccctcgacgctggagcccctctcctctgtgctggtatcacaacttacagtccactcaagtattttggactcgacaagcctggactaagggtgggtgtgaacggccttggtgggctcggtcatgtagctgtcaaattcgctaaggctttcgggactaaggtcactgtcatcagcacatctcttgccaagaaggaggaagctatacagcatcttggtgtggacgagtttgtagtcagcaccgatccacagcagatgcaggtacactgacaccactctcttctccccactttccctaaattcttcatcttcaatattaatcttttgtgtgttggtttggtaggctgcagtaggcacactagatggcatcattgacacggtctctgcaccgcatcccgttgttccgttgctgagcttgctgaagccacacgggaagcttatcgtggtcggccttccagataagcccctgcagcttcctgctttccccttgattcaaggtttgagttttcattacattcttatttatctgttatgtcttgttgaaagttggttatatatgtttgaggaaacaggtaggaagactgtggcggggagtggcattggagggcttaaggagacgcaagagatgattgatttcgcggccaagaacaacatagtagcggacgtggaggtcatccccatcgactatataaacacagcaatggatcggcttgatgtcaagtaccgtttcgtcatcgatgttgaaaaatccttgaaacctcagtga

SEQ ID NO: 61 - DNA, 1083 bp, Perilla setoyensis
atggcgaaaaccccagaaacagagcacccacttaaggcctttggttgggctgccagagacaattctgggaccttatctccattcaatttttctaggagggataccggtgagcgtgatgtgcagttcaaggtgttgtactgtggcgtttgtcattcagatctgcacatggtcaagaatgagtggggcgtaactcactacccggttgttcccgggcatgagattgtgggggttgtgacagaggtgggcagcaaggtcgaaaaggtgaaggccggggacaaagttggtgtgggagtcatagttggatcatgtcgccagtgtgatcagtgttccaacgatcttgaaaactactgctcgaagcaaatacttacctacggtgccccttacattgatggcaccattactcgtggaggttactctaacatcatggtggcagacgagcatttcattatccgttggcctgaaaacctgcctctcgatgctggagctcctctcctctgtgctggcatcacaacttacagtccattgaggtactatggactcgacaagcctggactaagtgttggtgtgaacggccttggtgggctgggtcatgtagccgtgaaatttgccaaagcttttgggactaaggtgactgttatcagcacatctcttggcaagaagaaggaagctattgaacatcttggtgtggatgagtttttggtcagtagtgacccgcagcagatgcaggctgcagtaggcacactagatggcatcattgacacagtctccgcagagcatcctcttgcgccgttgatgagcttgttgaagcctcatgggaagcttatcgtggttggagctccacaaaagcccctgcagcttcatgcttttagcctgatccaaggtaggaagacagtagcggggagtgggattggagggatgaaggagacgcaagagatgattgatttcgcggcaaagaagaacatcataccggatgtggaggtcatcccaatagactacataaacacagcaatggatcgccttctcaaatctgatgtcaagtacaggttcgtcattgatgttaacaaatctttcaatgctcactaa

SEQ ID NO: 62 - DNA, 1083 bp, Perilla citriodora
atggcgaaaaccccagaaacagagcacccagttaaggcctttggttgggctgccagagacaattctgggaccttatctccattcaatttttctaggagggataccggtgagcgtgatgtgcagttcaaggtgttgtactgtggcgtttgtcattcaggtctgcacatggtcaagaatgagtggggcgtaactcactacccggttgttcccggacatgagattgtgggggttgtgacagaggtgggcagcaaggtcgaaaaggtgaaggctggggacaaagttggtgtgggagtcatagttggatcatgtcgccagtgtgatcagtgttccaatgatcttgaaaactactgctcgaagcaaatacttacctacggtgccccttacattgatggcaccattactcgcggaggttactctaacatcatggtggcagacgagcatttcattatccgttggcctgaaaacctgcctctcgatgctggagctcctctcctctgtgctggcatcacaacttacagtccattgaggtactatggactcgacaagcctggactaagtgttggtgtgaacggccttggtgggctgggtcatgtagccgtgaaatttgccaaagcttttgggactaaggtgactgttatcagcacatctcttggcaagaagaaggaagctattgaacatcttggtgtggatgagtttttggtcagtagtgacccacagcagatgcaggctgcagtaggcacactagatggtatcattgacacagtctcagcagagcatccccttgtgccgttgatgagcttgttgaagcctcatgggaagcttatcgtggttggagctccagaaaagcccctgcagcttcatgcttttagcctgatccaaggtaggaagacagtagcggggagtgcgattggagggatgaaggagacgcaagagatgattgatttcgcggcaaagaagaacatcataccggatgtggaggtcatcccaatagattacataaacacagcaatggatcgccttctcaaatctgatgtcaagtacaggttcgtcattgatgttaacaaatctttcaatgctcactaa

SEQ ID NO: 63 - DNA, 1083 bp, Perilla frutescens
atggcgaaaaccccagaaacagagcacccagttaaggcctttggttgggctgccagagacaattctgggaccttatctccattcaatttttctaggagggataccggtgagcgtgatgtgcagttcaaggtgttgtactgtggcgtttgtcattcagatctgcacatggtcaagaatgagcggggcgtaactcactacccggttgttcccggacatgagattgtgggggttgtgacagaggtgggcagcaaggtcgaaaaggtgaaggctggggacaaagttggtgtgggagtcatagttggatcatgtcgccagtgtgatcagtgttccaatgatcttgaaaactactgctcgaagcaaatacttacctacggtgccccttacattgatggcaccattactcgcggaggttactctaacatcatggtggcagacgagcatttcattatccgttggcctgaaaacctgcctctcgatgctggagctcctctcctctgtgctggcatcacaacttacagtccattgaggtactatggactcgacaagcctggactaagtgttggtgtgaacggccttggtgggctgggtcatgtagccgtgaaatttgccaaagcttttgggactaaggtgactgttatcagcacatctcttggcaagaagaaggaagctattgaacatcttggtgtggatgagtttttggtcagtagtgacccacagcagatgcaggctgcagtaggcacactagatggtatcattgacacagtctcagcagagcatccccttgtgccgttgatgagcttgttgaagcctcatgggaagcttatcgtggttggagctccagaaaagcccctgcagcttcatgcttttagcctgatccaaggtaggaagacagtagcggggagtgcgattggagggatgaaggagacgcaagagatgattgatttcgcggcaaagaagaacatcataccggatgtggaggtcatcccaatagattacataaacacagcaatggatcgccttctcaaatctgatgtcaagtacaggttcgtcattgatgttaacaaatctttcaatgctcactaa

SEQ ID NO: 64 - DNA, 253 bp, Artificial sequence
  DNA sequence encoding nopaline synthase (NOS) teminator
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 65 - DNA, 401 bp, Artificial sequence
  DNA sequence encoding CaMV terminator
gaaatcaccagtctctctctacaaatctatctctctctattttctccataataatgtgtgagtagttcccagataagggaattagggttcttatagggtttcgctcacgtgttgagcatataagaaacccttagtatgtatttgtatttgtaaaatacttctatcaataaaatttctaattcctaaaaccaaaatccagtactaaaatccagatcacctaaagtccctatagatctttgtcgtgaatataaaccagacatgagacgactaaacctggagcccagacgccgttcgaagctagaagtaccgcttaggcaggaggccgttagggaaaagatgctaaggcagggttggttacgttgactcccccgtaggtttggtttaaatatgataaagtggac

SEQ ID NO: 66 - DNA, 270 bp, Artificial sequence
  DNA sequence encoding agropine synthase (AGS) terminator
tgcatttgattagtgaaaattaattttcagtgcagtattttctattcgatctttatgtaatccgtttcaatgaataaatttaaatccgatgattagctgttgtttatattaattcgtatttcatccatatttttattagaatcttgagatgttaaaaaatttcagtcaaaatttaaacatgaaactttattcaatatttctgtatcgcacaatgcaaatctatgtgagattacaagataccgacactgttatttaacataacatagacat

SEQ ID NO: 67 - DNA, 401 bp, Artificial sequence
  DNA sequence encoding octapin synthase (OCS) terminator
aatcaaatcttccagctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaa

SEQ ID NO: 68 - protein, 60 aa, Artificial sequence
  amino acids sequence encoding Chloroplast signal peptide of Arabidopsis ribulose bisphosphate carboxylase small chain
MASSMLSSAAVVTSPAQATMVAPFTGLKSSASFPVTRKANNDITSITSNGGRVSCMKVWP

SEQ ID NO: 69 - DNA, 426 bp, Artificial sequence
  DNA sequence encoding 35S promoter
caacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctg

SEQ ID NO: 70 - DNA, 301 bp, Artificial sequence
  DNA sequence encoding sgFiMV promoter
tttacagtaagaactgataacaaaaattttacttatttccttagaattaatcttaaaggtgatagtaaacaaggacgattagtccgttggcaaaattggttcagcaagtatcaatttgatgtcgaacatcttgaaggtgtaaaaaacgttttagcagattgcctcacgagagattttaatgcttaaaaacgtaagcgctgacgtatgatttcaaaaaacgcagctataaaagaagccctccagcttcaaagttttcatcaacacaaattctaaaaacaaaattttttagagagggggagtg

SEQ ID NO: 71 - DNA, 372 bp, Artificial sequence
  DNA sequence encoding SVBV promoter
gaagcccgctttacaagtggccagctagctatcactgaaaagacagcaagacaatggtgtctcgatgcaccagaaccacatctttgcagcagatgtgaagcagccagagtggtccacaagacgcactcagaaaaggcatcttctaccgacacagaaaaagacaaccacagctcatcatccaacatgtagactgtcgttatgcgtcggctgaagataagactgaccccaggccagcactaaagaagaaataatgcaagtggtcctagctccactttagctttaataattatgtttcattattattctctgcttttgctctctatataaagagcttgtattttcatttgaaggcagaggcgaacacacacacag

SEQ ID NO: 72 - DNA, 795 bp, Artificial sequence
  DNA sequence encoding NptII selection gene
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 73 - DNA, 21 bp, Artificial sequence
  Single strand DNA oligonucleotide
tggcacaagcactggatgttc

SEQ ID NO: 74 - DNA, 24 bp, Artificial sequence
  Single strand DNA oligonucleotide
aatctctgtcaactcgctctggtg

SEQ ID NO: 75 - DNA, 22 bp, Artificial sequence
  Single strand DNA oligonucleotide
gcaaaaaaggctaacaatcccc

SEQ ID NO: 76 - DNA, 23 bp, Artificial sequence
  Single strand DNA oligonucleotide
gaatgaatctcaacaccgtctgc

SEQ ID NO: 77 - DNA, 24 bp, Artificial sequence
  Single strand DNA oligonucleotide
ataaggttggtgtgggcgttctcg

SEQ ID NO: 78 - DNA, 23 bp, Artificial sequence
  Single strand DNA oligonucleotide
catcaagtgggaagttttcaggc

SEQ ID NO: 79 - protein, 361 aa, Gluconobacter oxydans
MPTLFDPIDFGPIHAKNRIVMSPLTRGRADKEAVPTPIMAEYYAQRASAGLIITEATGISREGLGWPFAPGIWSDAQVEAWKPIVAGVHAKGGKIVCQLWHMGRMVHSSVTGTQPVSSSATTAPGEVHTYEGKKPFEQARAIDAADISRILNDYENAARNAIRAGFDGVQIHAANGYLIDEFLRNGTNHRTDEYGGVPENRIRFLKEVTERVIAAIGADRTGVRLSPNGDTQGCIDSAPETVFVPAAKLLQDLGVAWLELREPGPNGTFGKTDQPKLSPQIRKVFLRPLVLNQDYTFEAAQTALAEGKADAIAFGRKFISNPDLPERFARGIALQPDDMKTWYSQGPEGYTDYPSATSGPN

SEQ ID NO: 80 - DNA, 1082 bp, Gluconobacter oxydans
atgcctaccctctttgaccctatcgattttgggcccatccacgccaagaataggatagtgatgagccctctcaccagagggagggccgataaggaggccgtgcctacccctatcatggccgaatactacgcccaaagggccagcgccggtctcatcatcactgaagccactgggatcagcagggagggtctcgggtggccttttgcccctgggatctggagcgacgctcaggttgaggcctggaagcccatcgtggccggtgtgcacgccaagggtgggaagatcgtgtgccaactctggcacatggggaggatggtgcacagcagcgttactgggactcaacctgtttcttctagcgccaccactgcccctggggaggtgcacacttatgaggggaagaagcctttcgagcaggccagggccatagacgccgccgatatcagcagaatcctcaatgactacgagaacgccgccaggaatgccatcagggccgggttcgatggggttcagatccacgccgccaatgggtacctcatcgatgagttcctcaggaatggtactaaccacaggaccgacgagtacgggggtgttcctgagaacaggatcaggttcctcaaggaggttaccgagagggtgatcgccgctataggtgccgataggactggtgtgaggcttagccctaatggtgacacccaagggtgcatcgacagcgcccctgagactgtgttcgttcctgccgccaagctccttcaagatctcggggtggcctggctcgagcttagggagcctgggcctaacgggacctttgggaagactgatcagcctaagctcagccctcaaatcaggaaggtgttcctcaggcctctcgtgctcaatcaggactacactttcgaggccgcccaaactgcccttgctgaggggaaggccgatgccatcgcctttgggaggaagttcatcagcaaccctgatctccccgagaggtttgccagggggatcgccctccaacccgacgacatgaaaacctggtacagccaagggcctgaggggtacaccgattacccttctgccactagcgggcctaa

SEQ ID NO: 81 - protein, 399 aa, Hevea brasiliensis
MAETGTEGTGITTLFSPYKMGKFSLSHRVVLAPMTRCRALNGIPNAALVDYYTQRSTPGGFLITEGTLVSPTAPGFPHVPGIYTEEQAEAWKRVVDAVHAKGSIIFCQLWHVGRASHQVYQPNGAAPISSTGKAISNRWRILMPDGSYGKYPTPRPLETPEILEVVKNYRQSALNAIRAGFDGIEVHGAHGYLIDQFLKDGINDRTDEYGGSINNRCRFLMQVIQAVVAAIGADRVGFRMSPAIDHLDAIDSDPLNLGLAVIERLNKLQLNLGSKLTYLHVTQPRYTAYGQTESGRHGTEEEEARLMRTWRRAYKGTFICSGGFTRELGMEAIAQDDADLVSYGRLFISNPDLVLRFKLNAPLNKYVRKTFYTQDPVVGYTDYPFFRKVDGSQEPRSRL

SEQ ID NO: 82 - DNA, 1191 bp, Hevea brasiliensis
atggctgagaccggtactgaagggactgggataaccactctcttctctccttacaagatggggaagtttagccttagccatagggtggtgctcgcccccatgaccaggtgcagggcccttaatgggatacccaatgccgccctcgtggactactacacccagaggagcactcctggggggttcctcatcactgaggggactcttgtgagccccaccgctcctggtttccctcacgtgcccggtatatacaccgaggaacaggccgaagcctggaagagagtggttgatgccgtgcacgccaaggggagcatcatcttttgccagctttggcacgtggggagggccagccaccaagtttaccaacctaatggggccgcccctatcagctctaccgggaaggctatcagcaacaggtggcgtattttaatgcccgacgggagctacgggaagtaccctacccctaggcctctcgagacccccgagatcctcgaggtggtgaagaactacaggcaaagcgccctcaatgccataagagccgggtttgatgggatagaggtgcatggggctcacggttatctcatcgatcagtttctcaaggacgggatcaacgacaggactgacgaatacggggggagcatcaacaataggtgcaggtttctcatgcaagttatacaagccgtggttgccgctatcggggctgatagggtgggttttaggatgagccctgctatcgaccaccttgatgccatcgacagcgaccctcttaatctcgggcttgccgtgatcgagagactcaataagctccagctcaacctcgggagcaaactcacctacctccacgtgacccagcctaggtacactgcctacgggcagaccgagtctgggagacacgggactgaggaggaggaggccaggcttatgaggacctggaggagggcctacaagggtacctttatctgtagcggggggtttaccagggaattagggatggaggccatcgcccaagacgatgccgacctcgtgagctacgggaggctctttatcagcaaccctgaccttgtgctcaggttcaagctcaacgcccccctcaataagtacgtgaggaagactttctacactcaggaccctgtggtggggtacaccgactaccccttctttaggaaggtggacgggagccaagagcctaggtga

SEQ ID NO: 83 - protein, 391 aa, Arabidopsis thaliana
MTAAQGNSNETLFSSYKMGRFDLSHRVVLAPMTRCRALNGVPNAALAEYYAQRTTPGGFLISEGTMVSPGSAGFPHVPGIYSDEQVEAWKQVVEAVHAKGGFIFCQLWHVGRASHAVYQPNGGSPISSTNKPISENRWRVLLPDGSHVKYPKPRALEASEIPRVVEDYCLSALNAIRAGFDGIEIHGAHGYLIDQFLKDGINDRTDQYGGSIANRCRFLKQVVEGVVSAIGASKVGVRVSPAIDHLDATDSDPLSLGLAVVGMLNKLQGVNGSKLAYLHVTQPRYHAYGQTESGRQGSDEEEAKLMKSLRMAYNGTFMSSGGFNKELGMQAVQQGDADLVSYGRLFIANPDLVSRFKIDGELNKYNRKTFYTQDPVVGYTDYPFLAPFSRL

SEQ ID NO: 84 - DNA, 1167 bp, Arabidopsis thaliana
atgaccgccgcccaggggaacagcaacgagaccctcttcagcagctacaagatggggaggttcgaccttagccatagagtggtgctcgcccccatgaccagatgtagagccctcaatggggtgcctaatgccgccctcgccgagtactacgctcagaggactactcctggtgggttccttataagcgaggggactatggtgagccctgggagcgccgggtttcctcacgtgcctggtatctactctgacgagcaggtggaagcctggaagcaggtggtggaggccgtgcacgccaagggggggtttatattttgccaactttggcacgtggggagagcctctcacgccgtgtaccaacctaacgggggtagccctatcagcagcaccaataagcctataagcgagaataggtggagggtgctcctccctgacgggagccacgtgaaataccctaagcctagggcccttgaggccagcgagatccctagggtggtggaggattactgccttagcgctctcaacgccataagggccggttttgacggtatcgagatccacggtgcccatgggtacctcatcgaccagttcctcaaggacggtatcaatgacaggactgaccagtacggggggagcatagccaacaggtgtaggttcctcaaacaagtggtggaaggggtggtttctgccatcggtgccagcaaggttggggtgagggttagccctgccatcgaccaccttgatgccaccgactctgaccctctcagccttgggctcgccgtggtggggatgctcaacaaattacagggggtgaacgggagcaagctcgcctacctccacgtgacccaacctaggtaccacgcctacgggcaaactgaatctgggagacaagggagcgacgaggaggaggctaagctcatgaagagcctcaggatggcctacaacgggaccttcatgagcagcggtgggttcaacaaggaattagggatgcaagctgtgcaacaaggtgatgccgatctcgtgagctacgggaggctcttcatagccaatcctgacctcgtgagcaggttcaagatcgacggggaattaaacaagtacaataggaagactttctatactcaagaccctgtggtggggtacactgactacccctttcttgcccctttctga

SEQ ID NO: 85 - protein, 396 aa, Solanum lycopersicum
MASSAQDGNNPLFSPYKMGKFNLSHRVVLAPMTRCRALNNIPQAALGEYYEQRATAGGFLITEGTMISPTSAGFPHVPGIFTKEQVREWKKIVDVVHAKGAVIFCQLWHVGRASHEVYQPAGAAPISSTEKPISNRWRILMPDGTHGIYPKPRAIGTYEISQVVEDYRRSALNAIEAGFDGIEIHGAHGYLIDQFLKDGINDRTDEYGGSLANRCKFITQVVQAVVSAIGADRVGVRVSPAIDHLDAMDSNPLSLGLAVVERLNKIQLHSGSKLAYLHVTQPRYVAYGQTEAGRLGSEEEEARLMRTLRNAYQGTFICSGGYTRELGIEAVAQGDADLVSYGRLFISNPDLVMRIKLNAPLNKYNRKTFYTQDPVVGYTDYPFLQGNGSNGPLSRL

SEQ ID NO: 86 - DNA, 1182 bp, Solanum lycopersicum
atggccagcagcgcccaagatgggaacaatcctctcttcagcccctacaagatggggaagttcaatctctctcacagggtggtgctcgcccctatgactaggtgcagggccctcaataacatacctcaggctgccctcggtgagtactacgagcaaagggccactgccggtgggttcctcatcaccgaggggactatgatctctcctaccagcgccgggttccctcatgtgcccggtatcttcactaaagaacaagtgagagagtggaagaagatagtggacgtggtgcacgccaagggggctgtgatcttttgccaactttggcacgttgggagggcttctcacgaggtgtatcaacctgccggggccgctcccatctcttctactgagaagcccatcagcaacaggtggagaatcctcatgcctgacggtactcacggtatataccctaagcctagggccatcgggacttatgagatcagccaggtggtggaggattacaggaggtctgccctcaacgctatcgaggccggttttgatgggatcgagatccacggggcccacgggtacctcatcgaccaattccttaaggacgggatcaatgataggactgatgagtacgggggtagcctcgccaataggtgcaagtttatcacccaggtggtgcaagccgtggtgagcgccataggggccgacagagtgggggtgagggtgagccctgccatcgatcatcttgacgccatggatagcaatcccctctctctcgggctcgccgtggtggagaggctcaacaagatccagctccacagcgggagcaagctcgcctaccttcatgtgactcagcctaggtacgtggcctacggtcagaccgaggctgggagactcgggagcgaggaggaagaggccaggctcatgaggactcttaggaatgcctaccaagggactttcatctgcagcggggggtacactagggaattagggatcgaggctgttgcccaaggggatgccgacctcgtgagctacgggaggctcttcatcagcaaccccgatctcgtgatgaggatcaagctcaacgctcctctcaataagtacaacagaaagaccttctacacccaagaccctgtggttgggtacactgattaccccttcctccaggggaatgggagcaatggtcctctttga

SEQ ID NO: 87 - protein, 389 aa, Eucalyptus grandis
MADSDLFTPYKMSKFDLSHRVVLAPMTRCRALGGIPRPAMAEYYAQRSTPGGFLISEGTMISDTAAGFPHVPGIYNEEQVRAWKKVVDSVHAKGSVIFCQLWHVGRASHQVYQPGGAAPLSSTDKPISQRWRILMPDGSHAIYPKPRALNHSDIQDMVEHYRQAAINAIRAGFDGIEIHGAHGYLIDQFLKDGINDRTDEYGGSIENRCRFIMQVVKAVAMAIRPDRVGVRMSPAIDHLDAMDSDPPKLGLAVIERLNKLQLELGSRLAYLHVTQPRYVAYGQTESGRLGSEEEEAQLMRTWRRAYQGTFICSGGFTRELGMKAIADGDADLVSYGRLFIANPDLVLRFQINAPLNKYVRKTFYTQDPVVGYTDYPFLDSRKQKLSSRL

SEQ ID NO: 88 - DNA, 1161 bp, Eucalyptus grandis
atggccgatagcgacctctttactccttacaagatgtctaagtttgacctcagccacagggttgtgctcgcccctatgactaggtgcagggctctcggtggtatccctaggcccgctatggccgaatactacgctcagagaagcacccctggggggttccttatctctgaggggaccatgatcagcgatactgctgccgggttccctcacgtgcctgggatctataacgaggagcaagtgagggcctggaagaaggtggtggattctgtgcatgccaaggggagcgtgatcttttgccaactctggcacgtggggagggcctctcaccaagtttaccaacctgggggggctgcccccctcagctctactgacaagcctatcagccagaggtggcgtattttaatgcccgacgggtctcacgccatctaccccaagcctagggccctcaaccacagcgacatccaggacatggtggagcactacaggcaggccgccatcaatgccatcagagccggttttgacggtatcgagatccatggggcccacgggtacctcatcgaccaattcctcaaggacgggatcaacgatagaaccgatgagtacggggggtctatagagaacaggtgcaggtttatcatgcaggtggtgaaggccgtggctatggctataaggcctgacagggtgggggtgagaatgagccctgccatcgatcacctcgacgccatggatagcgatcctcctaagctcgggctcgccgtgatcgagaggctcaataagctccagcttgaattagggtctaggcttgcctacctccatgtgacccaacctaggtacgttgcctacgggcaaactgaatctggtagactcgggagcgaggaggaggaagcccaactcatgaggacctggaggagggcctaccaggggacctttatctgcagcgggggtttcactagggaactcggtatgaaagctatcgccgatggggatgctgaccttgtgagctacgggaggctcttcatagctaatcctgacctcgtgctcaggtttcagatcaatgcccccctcaataagtacgtgaggaagaccttctacacccaagatcctgtggtggggtatactgactaccctttcctcgacagcagaaagcaaaaactcagctga

SEQ ID NO: 89 - protein, 393 aa, Rosa multiflora
MADSSVTLFSPIKMAKFNLSHRVVLAPMTRCRALNGVPNPALAEYYSQRSTPGGFLISEGTLVSDTAAGFPHVPGIFSDEQVEAWKKVVDAVHAKGAIIFCQLWHVGRASHGVYQPGGGSPISSTNNPISKRWKILLPDGSHGTYPQPQRLEIHEIPHIVEQYRRAAVNAIRAGFDGIEIHGAHGYLIDQFLKDGINDRTDEYGGSLANRCKFLIEIVQAVVEAIGADRVGVRVSPAIDHLDAMDSDPVSLGFAVIERLNKLQQNIGSKLTYLHVTQPRYAAYGQAESGKHGSDQDEAEFMRTLRKAYQGTFMASGGFTRELGMEAVASGDADLVSYGRYFISNPDLVLRFKLNAPLTKYVRKTFYTQDPVVGYTDYPFLNNTSIKEQAVSRL

SEQ ID NO: 90 - DNA, 1173 bp, Rosa multiflora
atggctgacagcagcgtgactctcttcagccccatcaagatggccaagttcaacctcagccacagagttgtgctcgcccctatgactaggtgcagggctctcaatggggtgcctaaccccgctctcgccgagtactactctcagaggagcactcctggggggttcctcataagcgaggggacccttgtgagcgatactgccgctgggtttcctcacgtgcctgggatctttagcgatgaacaagtggaggcctggaagaaggtggttgatgccgtgcacgccaagggtgccatcatcttctgccaactctggcatgtggggagggctagccatggggtgtaccaacctgggggggggagccctatcagcagcactaacaatcccatcagcaagaggtggaagatcctccttcctgacgggagccatgggacttacccccagcctcaaaggctcgaaatccacgagataccccacatcgtggagcaatacagaagggccgctgtgaatgccataagggccgggtttgatgggatcgagatccatggtgcccatgggtacctcatcgaccaatttctcaaggacgggatcaatgacaggaccgacgagtacggggggtctctcgccaacaggtgcaagttcctcatcgagatcgtgcaagccgtggtggaggccataggtgccgatagggttggggtgagggtgagccctgccatcgaccacctcgacgccatggatagcgaccccgttagcctcgggttcgccgttatcgagaggctcaataagctccaacaaaatatagggagcaagctcacctaccttcacgtgactcagcctaggtacgccgcctacgggcaagctgagagcggtaaacacgggtctgaccaagatgaagccgagttcatgaggaccctcaggaaggcttatcaagggaccttcatggcctctggggggtttactagggagcttggtatggaagctgtggctagcggggacgccgaccttgtgagctacgggagatactttatatctaaccctgaccttgtgctcaggttcaagctcaacgcccctctcactaagtacgtgaggaagaccttctacacccaagaccctgtggtggggtacaccgattacccttttctcaacaatactagcataaaggagcaagccgtgtga

SEQ ID NO: 91 - protein, 76 aa, Arabidopsis Chloroplast
MAQVSRICNGVQNPSLISNLSKSSQRKSPLSVSLKTQQHPRAYPISSSWGLKKSGMTLIGSELRPLKVMSSVSTAC